(19) United States
(12) Patent Application Publication
(10) Pub. No.: US 2011/0224105 A1
(43) Pub. Date: Sep. 15, 2011

(54) METHODS, COMPOSITIONS, AND KITS FOR GENERATING NUCLEIC ACID PRODUCTS SUBSTANTIALLY FREE OF TEMPLATE NUCLEIC ACID

(75) Inventors: Nurith Kurn, Palo Alto, CA (US); Shenglong Wang, San Ramon, CA (US)

(73) Assignee: NuGEN Technologies, Inc., San Carlos, CA (US)

(21) Appl. No.: 12/855,611

(22) Filed: Aug. 12, 2010

Related U.S. Application Data
(60) Provisional application No. 61/233,441, filed on Aug. 12, 2009.
(60) Provisional application No. 61/239,749, filed on Sep. 3, 2009.
(60) Provisional application No. 61/242,706, filed on Sep. 15, 2009.

(51) Int. Cl. C40B 50/06; C12P 19/34; C12N 9/24; C12N 9/12; C12Q 1/68

(52) U.S. Cl. 506/26; 435/91.5; 435/91.53; 435/91.2; 435/91.51; 435/200; 435/194; 435/6.1

(57) ABSTRACT

Methods, kits, and compositions are provided herein for the generation of double stranded DNA products suitable for downstream analysis.

### CROSS-REFERENCE

This application claims the benefit of U.S. Provisional Application No. 61/233,441, filed Aug. 12, 2009; U.S. Provisional Application No. 61/239,749, filed Sep. 3, 2009; and U.S. Provisional Application No. 61/242,706, filed Sep. 15, 2009, which applications are incorporated herein by reference in their entireties.

### BACKGROUND OF THE INVENTION

Large scale analysis of nucleic acids often requires amplification and further generation of products such as single stranded DNA (ss DNA) or double stranded DNA (ds DNA) that are either labeled, ready for labeling, or ready for ligation of desired adapters. These large scale analysis methods are evolving and comprise the use of various high or low density microarrays, high throughput sequencing, and other high throughput or highly parallel techniques. The limitations of current nucleic acid preparation techniques impact the ability to carry out large scale analysis of multiple parameters, as is required for, for example, the genotyping of multiple loci in the study of complex diseases, detecting the presence or absence of specific nucleic acid species in a sample, large scale sequencing and the like. Moreover, it is well accepted that molecular analysis determination of genomic instability in various pathological conditions such as cancer is most precisely carried out in well defined cell populations, such as those obtained by laser capture micro-dissection or cell sorting. Nucleic acid preparation technologies that provide robust amplification of very small polynucleotide samples, for example, from one or a very few cells, may provide a solution to the limited starting materials generally available for analysis.

Likewise, the ability to amplify ribonucleic acid (RNA) is an important aspect of efforts to elucidate biological processes. Total cellular mRNA represents gene expression activity at a defined time. Gene expression is affected by cell cycle progression, developmental regulation, response to internal and external stimuli and the like. The profile of expressed genes for any cell type in an organism reflects normal or disease states, response to various stimuli, developmental stages, cell differentiation, and the like, Non-coding RNAs have been shown to be of great importance in regulation of various cellular functions and in certain disease pathologies. Such RNAs are often present in very low levels. Thus, amplification methods capable of amplifying low abundance RNAs, as well as methods capable of amplifying whole transcriptome RNA are of great importance.

Therefore, there is a need for improved methods of preparing DNA and RNA samples, including methods which can globally or specifically amplify DNA or RNA polynucleotide targets. The invention described herein fulfills this need.

### SUMMARY OF THE INVENTION

Accordingly, the present invention provides improved methods for generation of ds DNA fragment molecules suitable for downstream applications including but not limited to large scale analysis.

In one embodiment, the present invention provides a method comprising: a) providing an input nucleic acid template (e.g. DNA or RNA); b) hybridizing one or more oligonucleotide primers to the input nucleic acid template; c) extending the one or more oligonucleotide primers along the input nucleic acid template with a polymerase comprising strand displacement activity, wherein the hybridizing and extending produces primer extension products comprising one or more double stranded products; and d) cleaving the input nucleic acid template with an agent comprising one or more cleavage reagents. In one aspect, steps (b) and (c) are performed simultaneously. In another aspect, steps (b) and (c) are performed sequentially. In a specific embodiment, the method further comprises generating one or more blunt ended double stranded products from the one or more double stranded products. The blunt ended double stranded products can be generated using enzymatic or non enzymatic methods. In the specific embodiment where enzymatic methods are utilized, the enzyme can be an exonuclease, an endonuclease, or a combination thereof. In the specific embodiment where the enzyme is an endonuclease, the endonuclease can be, but is not limited to, an S1 endonuclease, mung bean endonuclease, or a combination thereof. Such blunt ended double stranded products created using the methods provided herein, can be used to generate a library of blunt ended double stranded products. Such blunt ended double stranded products can be analyzed by next generation sequencing.

In a related embodiment, the present invention provides a method comprising: a) providing an input nucleic acid template (e.g., DNA or RNA) comprising one or more non-canonical nucleotides (e.g., dUTP) in a reaction mixture; b) hybridizing one or more oligonucleotide primers to the input nucleic acid template; c) extending the one or more oligonucleotide primers along the input nucleic acid template with a polymerase comprising strand displacement activity, wherein the hybridizing and extending produces primer extension products comprising one or more double stranded products; and d) cleaving the input nucleic acid template with an agent comprising one or more cleavage reagents. In one aspect, steps (b) and (c) are performed simultaneously. In another aspect, steps (b) and (c) are performed sequentially.

In a separate but related embodiment, the present invention provides a method comprising: a) contacting an input template nucleic acid (e.g., DNA or RNA) comprising one or more non-canonical nucleotides (e.g., dUTP) in a reaction mixture comprising: i) one or more oligonucleotide primers; ii) a polymerase comprising strand displacement activity; iii) an agent capable of cleaving a base portion of a non-canonical nucleotide, whereby an abasic site is generated; and iv) an agent capable of fragmenting a phosphodiester backbone at the abasic site; whereby double stranded DNA fragments are generated.

In a separate but related embodiment, the present invention provides a method comprising: a) providing an input nucleic acid template comprising one or more non-canonical nucleotides (e.g., dUTP); b) amplifying the input nucleic acid template to produce amplification products in a reaction mixture comprising oligonucleotide primers comprising random hybridizing portions, and an enzyme comprising strand displacement activity; and c) fragmenting the input nucleic acid template, wherein the fragmenting step is performed by adding to the reaction mixture comprising amplification products an agent that cleaves a base portion of a non-canonical nucleotide to generate an abasic site and an agent that cleaves a phosphodiester backbone of a nucleic acid at an abasic site. In one aspect of the embodiment, the method may further comprise d) optionally separating the amplification products from the fragmentation products; e) treating the amplification products with one or more agents to produce double stranded blunt-ended amplification products; and f) phosphorylating the 5′ ends of the double stranded blunt-ended amplification products. In one further aspect, step (d) is performed after step (f). In another further aspect, step (d) is performed before step (e). In yet another further aspect, step (d) is not performed. In another aspect of the embodiment, the method further comprises ligating the one or more phosphorylated double stranded blunt-ended amplification products with adaptor nucleic acid molecules. In some cases, the amplification products are analyzed by next generation sequencing.

In one aspect of any one of the foregoing embodiments, the present invention provides for cleaving a base portion of the non-canonical nucleotide, thereby forming an abasic site. In some cases the agent comprises a glycosylase such as UNG or UDG. In some cases, the agent comprises a primary amine, a polyamine, or DMED. In some cases, the agent is a glycosylase and a polyamine.

In one aspect of any one of the foregoing embodiments, the present invention provides a method wherein the one or more oligonucleotide primers comprise a label. For example, the one or more oligonucleotide primers may comprise an amino-allyl label.

In another aspect of any one of the foregoing embodiments, the one or more oligonucleotide primers comprise a random hexamer, heptamer, octomer, nonamer, decamer, undecamer, dodecamer, or tridecamer. In another aspect, the one or more oligonucleotide primers comprise a poly T sequence. In yet another aspect of any one of the foregoing embodiments, the one or more oligonucleotide primers comprise a mixture of random hexamers, heptamers, octomers, nonamers, decamers, undecamers, dodecamers, or tridecamers; and a poly T sequence.

In another aspect of any one of the foregoing embodiments, the method further comprises degrading single stranded DNA in the reaction mixture, such as with an ssDNA specific exonuclease, an ssDNA specific endonuclease, or a combination thereof. In some cases, the exonuclease is exonuclease 1, exonuclease 7 or a combination thereof. In some cases, the endonuclease is S1 endonuclease, mung bean endonuclease, or a combination thereof.

In another aspect of any one of the foregoing embodiments, the strand displacing polymerase comprises Klenow polymerase, exo-Klenow polymerase, exo-Klenow polymerase, Bst polymerase, Bst large fragment polymerase, Vent polymerase, Vent polymerase, Deep Vent (exo-) polymerase, 9° Nm polymerase, Therminator polymerase, Therminator II polymerase, MMulV Reverse Transcriptase, phi29 polymerase, or DyNAzyme EXT polymerase, or a combination thereof.

In another aspect of any one of the foregoing embodiments, the present invention further provides for phosphorylating the 5′ ends of the one or more blunt ended double stranded products.

In another aspect of any one of the foregoing embodiments, the present invention further provides for extending the 3′ end of the one or more blunt ended double stranded products.

In another aspect of any one of the foregoing embodiments, the present invention further provides for ligating the double stranded products with one or more double stranded adapter oligonucleotides.

In another aspect of any one of the foregoing embodiments, the present invention comprises providing the input nucleic acid template comprising one or more non-canonical nucleotides by performing PCR, strand displacement amplification (SDA), multiple displacement amplification (MDA), rolling circle amplification (RCR), single primer isothermal amplification (SPIA), Ribo-SPIA, ligase chain reaction (LCR), Nucleic Acid Sequence Based Amplification (NASBA), Q-Beta Replicase amplification, Self-sustained sequence replication (3SR) or ligation activated transcription (LAT).

In a separate but related embodiment, the present invention provides a kit comprising: a) a glycosylase; b) a polyamine, an AP endonuclease, or a combination thereof; and c) one or more double stranded adapter oligonucleotides. In one aspect of this embodiment, the present invention provides a kit comprising instructions for the use of said kit. In aspect, the instructions comprise a method for generating dsDNA fragments, said method comprising: a) providing an input nucleic acid template comprising one or more non-canonical nucleotides in a reaction mixture; b) hybridizing and extending one or more oligonucleotide primers to the input nucleic acid template with an enzyme comprising strand displacement activity, wherein the hybridizing and extending produces one or more double stranded products; and c) cleaving the input nucleic acid template.

In one aspect of the embodiment, the kit further comprises one or more oligonucleotide primers. In another aspect of this embodiment, the one or more oligonucleotide primers comprise random hybridizing portions. In another aspect of this embodiment, the one or more oligonucleotide primers comprise polyT hybridizing portions. In another aspect of this embodiment, the one or more oligonucleotide primers comprise a mixture of random hybridizing portions and polyT portions. In some cases, at least one or at least two of the one or more oligonucleotide primers is a composite primer.

In a separate but related embodiment, the present invention provides a composition comprising: a) an input template nucleic acid comprising one or more non-canonical nucleotides; b) one or more oligonucleotide primers comprising randomized hybridizing portions; c) a polymerase comprising strand-displacement activity; d) a glycosylase; and one or more double stranded DNA product molecules.

In another aspect, a kit is provided comprising: a) an input template nucleic acid, b) one or more oligonucleotide primers comprising randomized hybridizing portions; c) a polymerase comprising strand-displacement activity; and d) an endonuclease.

In another aspect, a method is provided comprising: a) providing an input RNA template in a reaction mixture; b) hybridizing a first primer to the input RNA template; c) reverse transcribing the input RNA template in the presence of one or more non-canonical nucleotides to generate a first strand cDNA; d) cleaving the input RNA template; e) performing second strand synthesis to generate a double stranded cDNA; and f) cleaving the first strand cDNA to generate a single stranded second strand cDNA.

In one embodiment, the reverse transcribing comprises use of an RNA-dependent DNA polymerase. In another embodiment, the reaction mixture further comprises an inhibitor of DNA-dependent DNA polymerase activity. In another embodiment, the inhibitor of DNA-dependent DNA polymerase activity inhibits the DNA-dependent DNA polymerase activity of the RNA-dependent DNA polymerase. In another embodiment, the inhibitor is actinomycin D. In another embodiment, the inhibitor is removed prior to the performing second strand synthesis. In another embodiment, the first primer comprises a 5′-tail sequence that does not hybridize to the input RNA template. In another embodiment, the 5′-tail sequence comprises DNA. In another embodiment, the 3′-end of the first primer hybridizes to the input RNA template. In another embodiment, the reverse transcribing comprises extension of the first primer hybridized to the input RNA template. In another embodiment, the non-canonical nucleotide is dUTP. In another embodiment, the second strand synthesis comprises primer extension of a second primer hybridized to the first strand cDNA. In another embodiment, the second primer comprises a 3′-sequence hybridizable to the first strand cDNA. In another embodiment, the second primer comprises a 5′-tail that is not complementary to the first strand cDNA. In another embodiment, the second strand synthesis is carried out by DNA polymerase. In another embodiment, the second strand synthesis is carried out in the absence of non-canonical nucleotide triphosphates. In another embodiment, cleaving the input RNA template in a complex with the first primer extension product or products comprises exposing the input RNA template to an RNase H with or without other RNases, or cleaving the input RNA template following first primer, extension reaction by heat or chemical treatment or combination thereof. In another embodiment, cleaving the first strand cDNA comprises combining the reaction mixture with an enzyme that cleaves the base portion of the non-canonical nucleotide to generate an abasic site.

In another embodiment, the enzyme that cleaves the base portion of the non-canonical nucleotide to generate an abasic site is a glycosylase. In another embodiment, the glycosylase is UNG or UDG. In another embodiment, the reaction mixture further comprises an amine. In another embodiment, the amine is DMED. In another embodiment, the cleaving the first strand cDNA generates fragments of the first strand cDNA with blocked 3′-ends.

In another embodiment, the method further comprises sequencing said single stranded second strand cDNA. In another embodiment, the sequencing comprises next generation sequencing. In another embodiment, the single stranded second strand cDNA comprises sequence homologous to the input RNA template flanked by 3′ and/or 5′ sequences compatible for use in DNA sequencing. In another embodiment, said single stranded second strand cDNA comprises sequence homologous to the input RNA template flanked by 3′ and/or 5′ comprising sequences that function as barcodes. In another embodiment, said single stranded second strand cDNA comprises sequence homologous to the input RNA template flanked by 3′ and/or 5′ sequences that comprise recognition sequence for one or more restriction enzymes. In another embodiment, said single stranded second strand cDNA comprises sequence homologous to the input RNA template flanked by 3′ and/or 5′ sequences that enables circularization by hybridizing an oligonucleotide complementary to the end sequences and ligation of the ends. In another embodiment, the method further comprises amplification of the cDNA by rolling circle amplification. In another embodiment, the method further comprises amplifying the single stranded cDNA by single primer isothermal amplification.

In another embodiment, the input RNA template is from a biological sample. In another the input RNA template is from a sample lysate. In another embodiment, the input RNA template is from a cell free fluid. In another embodiment, the cell free fluid is plasma or serum. In another embodiment, the input RNA template is fragmented RNA. In another embodiment, the input RNA template is from an FFPE sample. In another embodiment, the input RNA template is fragmented by treatment with heat in the presence of multivalent cations.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1a: depicts methods and compositions in one embodiment of the invention for generating double stranded nucleic acid products from one or more template polynucleotides utilizing a plurality of oligonucleotides comprising random hybridizing portions and a polymerase comprising strand displacement activity. In this embodiment the products are generated from a template that contains one or more non-canonical nucleotides (dUTP). The figure further depicts methods and compositions for fragmenting the template polynucleotide and removing undesired single stranded nucleic acids.

FIG. 1b: depicts methods and compositions in another embodiment of the invention for generating double stranded nucleic acid products from one or more template polynucleotides utilizing a plurality of oligonucleotides comprising random hybridizing portions and a polymerase comprising strand displacement activity. In this embodiment the template nucleotide does not contain any non-canonical nucleotides. Methods and compositions for fragmenting the template polynucleotide and removing undesired single stranded nucleic acids can be used.

FIG. 2: depicts methods and compositions for preparing double stranded nucleic acid for adaptor ligation.

FIG. 3: depicts methods and compositions for generating labeled double stranded nucleic acid products from one or more template polynucleotides utilizing a plurality of oligonucleotides comprising one or more labels and random hybridizing portions and a polymerase comprising strand displacement activity. The figure further predicts methods and compositions for fragmenting the template polynucleotide. Undesired single stranded nucleic acids can be removed.

FIG. 4: depicts BioAnalyzer profiles of input amplified cDNA, random primed products mixture and random primed products following cleavage of the input amplified cDNA. Whole transcriptome amplified cDNA from HeLa total RNA. The x-axis represents product length in bp. The concentration of the starting material is 172 ng/μL. The concentration of random primed amplified DNA is 200 ng/μL. The concentration of the random primed and fragmented input nucleic acid is 100 ng/μL.

FIG. 5: depicts BioAnalyzer profiles of input amplified cDNA, random primed products mixture and random primed products following cleavage of the input amplified cDNA. Whole transcriptome amplified cDNA from total RNA from a biological sample and brain. The x-axis represents product length in bp. The profile of the random priming products following removal of the input amplified cDNA is very reproducible for the two samples which represent different sample source.

FIG. 6: depicts an individual using a kit to prepare a blunt-ended sample from a whole transcriptome which is then put onto a next-generation nucleic acid analysis platform, such as a high-throughput sequencing apparatus. The performing and monitoring of the sequencing reactions may be automated such that the sequencing reactions are performed via robotics. In addition, the sequencing information and data obtained may be provided to a personal computer, a personal digital assistant, a cellular phone, a video game system, or a television so that a user can monitor the progress of the sequencing reactions remotely.

FIG. 7: illustrates the generation of second strand cDNA with defined sequences at 3′- and 5′-ends.

FIG. 8: illustrates that second strand cDNA with defined 5′- and 3′-ends can be amplified with single primer isothermal amplification (SPIA).

### DETAILED DESCRIPTION OF THE INVENTION

### I. Introduction

Large scale analysis of nucleic acids often requires amplification and generation of products which are single stranded (ss DNA or RNA) or double stranded (ds DNA) and are either labeled, ready for labeling, ready for ligation of desired adaptors and the like. These large scale analysis methods are evolving and comprise, e.g. pyrosequencing, sequencing by synthesis, sequencing by hybridization, single molecule sequencing, nanopore sequencing, and sequencing by ligation, high density PCR, microarray hybridization, array comparative genomic hybridization (CGH), serial analysis of gene expression (SAGE), digital PCR, and massively parallel Q-PCR; subtractive hybridization; differential amplification; preparation of libraries (including cDNA and differential expression libraries); preparation of an immobilized nucleic acid (which can be a nucleic acid immobilized on a microarray), and characterizing amplified nucleic acid products generated by the methods of the invention.

Various methods for global amplification of DNA target molecules (e.g., whole genome amplification) have been described, including methods based on the polymerase chain reaction (PCR). See, e.g., U.S. Pat. Nos. 5,731,171; 6,365,375; Daigo et al., (2001) Am. J. Pathol. 158 (5):1623-1631; Wang et al, (2001); Cancer Res. 61:4169-4174; Zheng et al, (2001) Cancer Epidemiol. 10:697-700; Dietmaier et al (1999) Am. J. Pathol. 154 (1) 83-95; Stoecklein et al (2002) Am. J. Pathol. 161 (1):43-51; U.S. Pat. Nos. 6,124,120; 6,280,949; Dean et al (2002) PNAS 99 (8):526′-5266. However, PCR-based global amplification methods, such as whole genome amplification (WGA), may generate non-specific amplification artifacts, give incomplete coverage of loci, or generate DNA of insufficient length that cannot be used in many applications. PCR-based methods also suffer from the propensity of the PCR reaction to generate products that are preferentially amplified, and thus resulting in biased representation of genomic sequences in the products of the amplification reaction. Methods of global amplification of DNA using composite primers have been described. See, e.g. U.S. patent application Ser. No. 10/824,829.

Additionally, a number of methods for the analysis of gene expression have been developed in recent years. See, for example, U.S. Pat. Nos. 6,251,639; 6,692,918; 6,686,156; 5,744,308; 6,143,495; 5,824,517; 5,829,547; 5,888,779; 5,545,522; 5,716,785; 5,409,818; EP 0971039A2; EP0878553A2; and U.S. Patent Application Publication Nos. 2002/0115088, 2003/0186234, 2003/0087251, and 2004/0023271. These include quantification of specific mRNAs and the simultaneous quantification of a large number of mRNAs as well as the detection and quantification of patterns of expression of known and unknown genes. RNA amplification is most commonly performed using the reverse transcriptase-polymerase chain reaction (RT-PCR) method and variations thereof. These methods are based on replication of RNA by reverse transcriptase to form single stranded DNA complementary to the RNA (cDNA), which is followed by polymerase chain reaction (PCR) amplification to produce multiple copies of double stranded DNA. RNA amplification also includes methods based on in vitro transcription such as, for example, SP6, T3, or T7 transcription methods. These methods are based on replication of RNA by reverse transcriptase to form DNA (cDNA), which may then include an optional amplification step such as any of the amplification steps provided herein including, but not limited to, PCR or linear amplification. Multiple copies of the RNA can then be made using a DNA dependent RNA polymerase such as T7, T3 or SP6 polymerase which acts on a promoter that has been incorporated during the step of reverse transcription or during a subsequent amplification step. Methods of amplification from RNA templates have been described, for example, in U.S. Patent Application Publication Nos. 2003/0096349, 20040081978 and U.S. Pat. Nos. 5,989,812 and 6,946,251.

Sequencing of nucleic acids continues to be a useful way to analyze DNA and RNA samples. Recent developments have made possible highly parallel high throughput sequencing. Many of these approaches use an in vitro cloning step to generate many copies of each individual molecule. Emulsion PCR is one method, which can involve isolating individual DNA fragment molecules along with primer-coated beads in aqueous bubbles within an oil phase. A polymerase chain reaction (PCR) then coats each bead with clonal copies of the isolated library molecule and these beads are subsequently immobilized for later sequencing. See, e.g., WO04069849A2 and WO05010145A2. In other cases, surface methods of clonal amplification have been developed, for example, by the use of bridge PCR where fragments are amplified upon primers attached to a solid surface. In some cases, bridge PCR requires the ligation of nucleic acid adaptor molecules to double stranded fragment molecules to allow efficient capture of the fragments on the solid surface. These methods produce many physically isolated locations which each contain many copies of a single fragment. While these methods have provided improvements in sequencing throughput, there is a continuing need to improve the methods of obtaining samples appropriate for sequencing, and of handling, and amplifying such samples to produce suitable double or single stranded nucleic acid. In particular, there is a need to improve methods for obtaining suitable nucleic acid product molecules from complex samples such as whole genome or transcriptome samples, or samples comprising a large number of genomic fragments or RNA fragments.

The generation of the ds DNA products may be based on random priming of the target nucleic acids, which may represent whole genome, whole transcriptome, total RNA or total DNA in a sample. Random primers and corresponding DNA polymerases suitable for this reaction are provided herein. Additional random primers and suitable polymerases are known in the art. Random hexamers, nonamers and longer oligonucleotide primers are suitable for the methods of the present invention, as well as various combinations of random, not-so random, and non random sequences. The primers may be labeled, may contain components which enhance hybridization, groups or links which prevent degradation by a proof reading polymerase and other modification designed for the specific applications. The generation of the ds DNA products may also be based on non-random priming or “not so random” priming of the target nucleic acids in a sample. For example, a pool of primers may be utilized that randomly hybridizes to total mRNA or a substantial fraction thereof but does not substantially hybridize to rRNA. Alternatively, a pool of primers may be utilized that hybridizes to a large number of genomic sequences, while not hybridizing to undesired genomic sequences. Non random primers which are designed to randomly prime along the desired template nucleic acids in the sample have been described for example in international patent application publication number WO 2009/055732.

DNA polymerases useful for random primed DNA synthesis can be selected from a group comprising polymerases with strand displacement activity. Both thermostable and thermolabile polymerases useful for these reactions include Klenow polymerase, with or without 3′-exonuclease, Bst DNA polymerase, φ29 DNA polymerase and other polymerases known in the art. In some cases, the polymerase does not comprise a 5′-exonuclease activity.

Random priming of the input nucleic acid template with a DNA polymerase comprising strand displacement activity may result in priming along the template as well as the displaced single stranded primer extension products leading to the generation of ds DNA products comprising primer extension products as well as ds DNA species comprising the original template, or templates, and a complement primer extension primer. Thus the products of random priming reaction as described above are non homogenous with respect to their origin, that is to say, a mixture of the input nucleic acid template and primer extension products, the last comprise both ds DNA and single stranded DNA. The non homogeneous nature of the mixture of nucleic acids at the end of the random priming reaction with a polymerase comprising stand displacement activity also includes differences in size, in so far as the input nucleic acid is longer. Additionally, the non homogenous nature may include differences in composition; for example, the random priming product may be synthesized in the presence of modified nucleotide tri-phosphates such as labeled nucleotide tri phosphate, when random priming is carried out for the generation of labeled products, such as required for example for array based analysis.

It is desirable to generate a homogenous population of random priming products for downstream analysis, such as array based analysis, for example, array based comparative genome analysis (array CGH), or next generation high throughput sequencing (for example Illumina Genome Analysis). In the first example random priming is carried out for the generation of labeled target libraries from the respective test and reference genomic DNA. Only the random priming products are labeled by incorporation of labeled dNTP or employing a labeled random primer, while the input nucleic acid to be analyzed is not labeled. It is desirable to separate the non labeled, much longer, input nucleic acid from the labeled shorter random priming products for improved analysis. For example, the longer input nucleic acid may provide cross hybridization resulting in erroneous results. Additionally, it is possible that the determination of label specific activity of the random priming products applied to the array will be inaccurately determined in a mixture of labeled random priming products and non labeled input nucleic acid.

Similarly, the generation of ds DNA libraries from nucleic acid to be sequenced using the newly developed second generation high throughput sequencing platforms, such as the commercially available Genome Analyzer (Illumina), will benefit from increasing the homogeneity of the random priming products used for ligation dependent adaptor ligation required for subsequent clonal amplification (cluster formation).

Large scale analysis of biological samples is often limited by the scarcity of the nucleic acid, RNA or DNA, samples. The amount of nucleic acids in biological samples is often much lower than that required for large scale analysis. The increased recognition of the importance of analyzing defined cell populations in biological samples, as little as single cells, imposes further limitation of direct analysis of the sample nucleic acid. Thus, the genome wide analysis of biological samples benefits from the development of methods and reagents for linear amplification of the sample nucleic acids (RNA and/or DNA). The present invention provides methods, composition and kits for generation of linearly amplified products which are specifically marked to be employed as templates for target generation by, for example, random priming, and to be distinguished from the random priming products described, for further fragmentation and removal from the desired products. The methods are also applicable to other amplification procedures and reagents. In general, the methods of the invention apply to any method in which the sample nucleic acid is first replicated or amplified employing DNA synthesis in the presence of non canonical dNTP for marking of the synthesized nucleic acid which will in turn serve as an input for further preparation of targets for downstream analysis, such as generation of double stranded or single stranded products by random priming. In some cases, the replication or amplification is performed in a limited fashion such as a single round of amplification or a small number of rounds of amplification (e.g., 1, 2, 3, 4, 5, 6, 7, 8, 9, 10, 15, 20, 25, 30, 35, 40 etc.) such as may be used during the synthesis of cDNA to avoid amplification bias. In other cases, the invention provides methods for generating a first copy of the template RNA in the sample, employing an RNA dependent DNA polymerase, in the presence of non canonical nucleotide and an inhibitor of DNA dependent DNA polymerase, thus generating a marked first strand cDNA which can serve as the input for generating second strand cDNA by DNA dependent DNA polymerase in the absence of the non canonical nucleotides and the inhibitor of DNA dependent DNA polymerase, thus generating first and second cDNA strands which are distinguishable by the presence of the non canonical nucleotides in the said first but not second strand cDNA. The said first strand cDNA can be degraded by the methods of the inventions (fragmentation by glycosylase and DMED for example) without affecting the second strand cDNA which can be subjected to down stream analysis such highly parallel sequencing in a strand specific manner.

### II. Exemplary Methods of the Invention

The methods of the present invention may include a step of providing an input nucleic acid template in a reaction mixture. Suitable input nucleic acid templates include, but are not limited to, e.g., DNA or RNA templates. In some embodiments the templates do not comprise non-canonical nucleotides. In other embodiments the templates comprise one or more non-canonical nucleotides. The input nucleic acid template may comprise DNA or RNA or a combination thereof, which template may optionally further comprise one or more non-canonical nucleotides. In some cases, the input nucleic acid template is DNA such as, for example, whole genomic DNA or a portion thereof. In some cases, the input nucleic acid template is DNA that has been previously selected. For example DNA, such as genomic DNA, may be hybridized to capture probes (e.g., capture probes on an array or on beads), washed to remove un-hybridized sequences corresponding to undesired fragments of nucleic acid and then collected for the methods of the present invention. Alternatively, DNA may be previously selected by non-random or “not so random” priming and amplification of a sample of genomic DNA. In some cases, the non-random or not-so random priming and amplification may be performed in the presence of one or more non-canonical nucleotides such that they are incorporated into the template nucleic acid. In some cases the DNA derived from the RNA is generated by reverse transcriptase in the presence of non canonical nucleotide (e.g., dUTP) and an inhibitor of the DNA dependent DNA polymerase activity of the reverse transcriptase (e.g., actinomycin D). In related cases, the non-random or not-so-random priming and amplification may be performed not in the presence of non-canonical nucleotides. In some cases the template is RNA such as total RNA, mRNA, viral RNA, small RNA fraction and the like. In other cases, the input nucleic acid template may comprise DNA that is derived from RNA such as by reverse transcription. Alternatively, the input nucleic acid template may comprise RNA that is derived from DNA such as by in vitro transcription. In some cases, the reverse transcription or in vitro transcription may be performed in the presence of one or more non-canonical nucleotides which are thus incorporated into the template nucleic acid. In other cases, non-canonical nucleotides are not incorporated into the template nucleic acid. The nucleic acid in the sample may also represent a mixture of genomic DNAs, such as from a microbial population.

Reaction mixtures suitable for the methods of the present invention include aqueous solutions or water in oil emulsions as provided herein. Various buffers, detergents, salts, chelators, chaotropes, azotropes, solvents, cations, divalent cations such as magnesium or manganese, and anions are suitable for storage and downstream manipulation (e.g., hybridization, polymerization, etc.) of nucleic acids. Reaction mixture compositions suitable for the methods of the present invention are provided herein.

In some cases, the input nucleic acid template is RNA, such as for example, whole transcriptome RNA, or a portion thereof (e.g., mRNA or miRNA). In some cases the input nucleic acid template is RNA that does not comprise any non-canonical nucleotides. In some cases, the input nucleic acid template is RNA that further comprises one or more non-canonical nucleotides. In some cases, the input nucleic acid template is RNA that has been previously selected. For example, RNA may be hybridized to capture probes (e.g., capture probes on an array), washed to remove un-hybridized sequences corresponding to undesired fragments of RNA and then collected for the methods of the present invention. Alternatively, RNA may be previously selected by non-random or “not so random” priming and amplification of a sample of RNA. In some cases, the RNA may be previously selected by non-random or not so random priming and amplification in the presence of one or more non-canonical nucleotides such that they are incorporated into the template nucleic acid.

The input nucleic acid template may be provided by first performing an amplification reaction on a sample comprising nucleic acid. In some cases, the amplification reaction may be performed in the presence of a non-canonical nucleotide. In some cases, the amplification reaction may be performed not in the presence of a non-canonical nucleotide. Conditions for limited and/or controlled incorporation of a non-canonical nucleotide are provided herein. See, e.g., Jendrisak, U.S. Pat. No. 6,190,865; Mol. Cell Probes (1992) 251-6; Anal. Biochem. (1993) 211:164-9; see also Sambrook (1989) “Molecular Cloning: A Laboratory Manual”, second edition; Ausebel (1987, and updates) “Current Protocols in Molecular Biology.” The frequency (or spacing) of non-canonical nucleotides in the resulting polynucleotide comprising a non-canonical nucleotide, and thus the average size of fragments generated using the methods of the invention (i.e., following cleavage of a base portion of a non-canonical nucleotide, and cleavage of a phosphodiester backbone at a non-canonical nucleotide), is controlled by variables including: frequency of nucleotide(s) corresponding to the non-canonical nucleotide(s) in the template (or other measures of nucleotide content of a sequence, such as average G-C content), ratio of canonical to non-canonical nucleotide present in the reaction mixture; ability of the polymerase to incorporate the non-canonical nucleotide, relative efficiency of incorporation of non-canonical nucleotide verses canonical nucleotide, and the like. It is understood that the average fragmentation size also relates to the reaction conditions used during fragmentation, as is further discussed herein. The reaction conditions can be empirically determined, for example, by assessing average fragment size generated using the methods of the invention taught herein. The level of labeling at an abasic site also relates to the frequency of incorporation of non-canonical nucleotides, as is further discussed herein.

In the embodiment where the template nucleic acid contains one or more non-canonical nucleotides, the template nucleic acid comprising one or more non-canonical nucleotides may be synthesized so that it comprises a sufficient and predictable density of non canonical nucleotides to provide for sufficient and predictable fragmentation, and when used with one or more agents capable of cleaving at the non-canonical nucleotides (e.g., a glycosidase, a glycosidase and an amine, or a glycosidase and an AP endonuclease) to further generate fragments of desirable size range. In some cases, the one or more agents employed for cleaving at the non-canonical nucleotides of the template nucleic acid may provide fragmentation products with a blocked 3′-end. Generally, a non-canonical base can be incorporated at about every 5, 10, 15, 20, 25, 30, 40, 50, 65, 75, 85, 100, 123, 150, 175, 200, 225, 250, 300, 350, 400, 450, 500, 550, 600, 650 or more nucleotides apart in the resulting polynucleotide comprising a non-canonical nucleotide. In one embodiment, the non-canonical nucleotide is incorporated about every 200 nucleotides, about every 100 nucleotide, or about every 50, 25, 20, 15, 10, 9, 8, 7, 6, 5, or fewer nucleotides. In another embodiment, the non-canonical nucleotide is incorporated about every 50 to about 200 nucleotides. In some embodiments, a 1:1, 1:2, 1:3, 1:4, 1:5, 1:6, 1:10, 1:15, 1:20 or higher ratio of non-canonical to canonical nucleotide may be used in the reaction mixture. In some cases, a 1:1, 1:2, 1:3, 1:4, 1:5, 1:6, 1:10, 1:15, 1:20 or higher ratio of the non-canonical nucleotide dUTP to canonical nucleotide dTTP is used in the reaction mixture.

The input nucleic acid template or the nucleic acid starting material from which template nucleic acid comprising one or more non-canonical nucleotides is provided for the methods of the present invention may include double-stranded, partially double-stranded; and single-stranded nucleic acids from any source in purified or unpurified form, which can be DNA (dsDNA and ssDNA) or RNA, including tRNA, mRNA, rRNA, mitochondrial DNA and RNA, chloroplast DNA and RNA, DNA-RNA hybrids, or mixtures thereof, genes, chromosomes, plasmids, the genomes of biological material such as microorganisms, e.g., bacteria, yeasts, viruses, viroids, molds, fungi, plants, animals, humans, and fragments thereof. Obtaining and purifying nucleic acids use standard techniques in the art. RNAs can be obtained and purified using standard techniques in the art. Starting material comprising DNA (including genomic DNA) can be transcribed into RNA form, which can be achieved using methods disclosed in Kurn, U.S. Pat. No. 6,251,639, and by other techniques, such as expression systems. RNA copies of genomic DNA would generally include untranscribed sequences generally not found in mRNA, such as introns, regulatory and control elements, etc. DNA copies of an RNA starting material can be synthesized using methods described in Kurn, U.S. Patent Publication No. 2003/0087251 or other techniques known in the art.

Synthesis of template polynucleotide comprising one or more non-canonical nucleotides from a DNA-RNA hybrid can be accomplished by for example denaturation of the hybrid to obtain ssDNA and/or RNA, cleavage with an agent capable of cleaving RNA from an RNA/DNA hybrid. The nucleic acid starting material can be only a minor fraction of a complex mixture such as a biological sample and can be obtained from various biological materials by procedures well known in the art. The starting material nucleic acid can be known or unknown and may contain more than one desired specific nucleic acid sequence of interest, each of which may be the same or different from each other. Therefore, the methods of the invention are useful not only for producing one specific polynucleotide suitable for downstream analysis, but also for producing simultaneously more than one different specific polynucleotides from one or more than one different template nucleic acid comprising a non-canonical nucleotide. The nucleic acid starting material can be a sub-population of nucleic acids, for example, a subtractive hybridization probe, total genomic DNA, restriction fragments, a cDNA library, cDNA prepared from total mRNA, a cloned library, or amplification products of any of the templates described herein. In some cases, the initial step of the synthesis of the complement of a portion of a nucleic acid starting material sequence is template denaturation. The denaturation step may be thermal denaturation or any other method, such as alkali treatment.

For simplicity, the template polynucleotide either comprising a non-canonical nucleotide or not comprising a non-canonical nucleotide is described as a single nucleic acid. It is understood that the polynucleotide can be a single polynucleotide, or a population of polynucleotides (from a few to a multiplicity to a very large multiplicity of polynucleotides). It is further understood that a template polynucleotide can be a multiplicity (from small to very large) of different polynucleotide molecules. Such populations can be related in sequence (e.g., member of a gene family or superfamily) or extremely diverse in sequence (e.g., generated from all mRNA, generated from all genomic DNA, etc.). Polynucleotides can also correspond to single sequence (which can be part or all of a known gene, for example a coding region, genomic portion, etc.). Methods, reagents, and reaction conditions for generating specific polynucleotide sequences and multiplicities of polynucleotide sequences are known in the art.

Suitable methods of synthesis of a polynucleotide comprising a non-canonical nucleotide are generally template-dependent (in the sense that polynucleotide comprising a non-canonical nucleotide is synthesized along a polynucleotide template, as generally described herein). It is understood that non-canonical nucleotides can be incorporated into a polynucleotide as a result of template-independent methods. For example, one or more primer(s) can be designed to comprise one or more non-canonical nucleotides. See, e.g., Richards, U.S. Pat. Nos. 6,037,152, 5,427,929, and 5,876,976. As discussed herein, inclusion of at least one non-canonical nucleotide in a primer results in cleavage of a base-portion of a non-canonical nucleotide and labeling at the abasic site (i.e., following generation of an abasic site, as described herein), thus generating a polynucleotide fragment comprising a portion of the primer.

Suitable amplification reactions further include any DNA or RNA amplification reaction, including but not limited to polymerase chain reaction (PCR), strand displacement amplification (SDA), multiple displacement amplification (MDA), rolling circle amplification (RCR), single primer isothermal amplification (SPIA, see e.g. U.S. Pat. No. 6,251,639), RNA single primer isothermal amplification (Ribo-SPIA, see e.g. US Patent Application Publication No. 2003/0087251), ligase chain reaction (LCR), nucleic acid sequence based amplification (NASBA), Q-beta replicase amplification, self-sustained sequence replication (3SR), ligation activated transcription (LAT), in vitro transcription (IVT), or a combination thereof. In some cases, the amplification methods for providing the template nucleic acid may be performed under limiting conditions such that only a few rounds of amplification (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 etc.), such as for example as is commonly done for cDNA generation. The number of rounds of amplification can be about 1-30, 1-20, 1-15, 1-10, 5-30, 10-30, 15-30, 20-30, 10-30, 15-30, 20-30, or 25-30.

PCR is an in vitro amplification procedure based on repeated cycles of denaturation, oligonucleotide primer annealing, and primer extension by thermophilic template dependent polynucleotide polymerase, resulting in the exponential increase in copies of the desired sequence of the polynucleotide analyte flanked by the primers. The two different PCR primers, which anneal to opposite strands of the DNA, are positioned so that the polymerase catalyzed extension product of one primer can serve as a template strand for the other, leading to the accumulation of a discrete double stranded fragment whose length is defined by the distance between the 5′ ends of the oligonucleotide primers.

LCR uses a ligase enzyme to join pairs of preformed nucleic acid probes. The probes hybridize with each complementary strand of the nucleic acid analyte, if present, and ligase is employed to bind each pair of probes together resulting in two templates that can serve in the next cycle to reiterate the particular nucleic acid sequence.

NASBA is a promoter-directed, enzymatic process that induces in vitro continuous, homogeneous and isothermal amplification of a specific nucleic acid to provide RNA copies of the nucleic acid. The reagents for conducting NASBA include a first DNA primer with a 5′-tail comprising a promoter, a second DNA primer, reverse transcriptase, RNAse-H, T7 RNA polymerase, NTP's and dNTP's.

The Q-beta-replicase method relies on the ability of Q-beta-replicase to amplify its RNA substrate exponentially. The reagents for conducting such an amplification include “midi-variant RNA” (amplifiable hybridization probe), NTP's, and Q-beta-replicase.

3SR is similar to NASBA except that the RNAse-H activity is present in the reverse transcriptase. Amplification by 3SR is an RNA specific target method whereby RNA is amplified in an isothermal process combining promoter directed RNA polymerase, reverse transcriptase and RNase H with target RNA. See for example Fahy et al. PCR Methods Appl. 1:25-33 (1991).

TMA, used by Gen-Probe, is similar to NASBA in utilizing two enzymes in a self-sustained sequence replication. See U.S. Pat. No. 5,299,491 herein incorporated by reference.

SDA (Westin et al 2000, Nature Biotechnology, 18, 199-202; Walker et al 1992, Nucleic Acids Research, 20, 7, 1691-1696), is an isothermal amplification technique based upon the ability of a restriction endonuclease such as HincII or BsoBI to nick the unmodified strand of a hemiphosphorothioate form of its recognition site, and the ability of an exonuclease deficient DNA polymerase such as Klenow exo minus polymerase, or Bst polymerase, to extend the 3′-end at the nick and displace the downstream DNA strand. Exponential amplification results from coupling sense and antisense reactions in which strands displaced from a sense reaction serve as targets for an antisense reaction and vice versa.

RCA (Lizardi et al. 1998, Nature Genetics, 19:225-232) can be used to amplify single stranded molecules in the form of circles of nucleic acids. In its simplest form, RCA involves the hybridization of a single primer to a circular nucleic acid. Extension of the primer by a DNA polymerase with strand displacement activity results in the production of multiple copies of the circular nucleic acid concatenated into a single DNA strand.

In some embodiments of the invention, RCA is coupled with ligation. For example, a single oligonucleotide can be used both for ligation and as the circular template for RCA. This type of polynucleotide can be referred to as a “padlock probe” or a “RCA probe.” For a padlock probe, both termini of the oligonucleotide contain sequences complementary to a domain within a nucleic acid sequence of interest. The first end of the padlock probe is substantially complementary to a first domain on the nucleic acid sequence of interest, and the second end of the padlock probe is substantially complementary to a second domain, adjacent to the first domain near the first domain. Hybridization of the oligonucleotide to the target nucleic acid results in the formation of a hybridization complex. Ligation of the ends of the padlock probe results in the formation of a modified hybridization complex containing a circular polynucleotide. In some cases, prior to ligation, a polymerase can fill in the gap by extending one end of the padlock probe. The circular polynucleotide thus formed can serve as a template for RCA that with the addition of a polymerase results in the formation of an amplified product nucleic acid. The methods of the invention described herein, can produce amplified products with defined sequences on both the 5′- and 3′-ends. Such amplified products can be used as padlock probes.

Some aspects of the invention utilize linear amplification of nucleic acids or polynucleotides. Linear amplification generally refers to a method that involves the formation of one or more copies of the complement of only one strand of a nucleic acid or polynucleotide molecule, usually a nucleic acid or polynucleotide analyte. Thus, the primary difference between linear amplification and exponential amplification is that in the latter process, the product serves as substrate for the formation of more product, whereas in the former process the starting sequence is the substrate for the formation of product but the product of the reaction, i.e. the replication of the starting template, is not a substrate for generation of products. In linear amplification the amount of product formed increases as a linear function of time as opposed to exponential amplification where the amount of product formed is an exponential function of time.

In some embodiments, amplification methods can be solid-phase amplification, polony amplification, colony amplification, emulsion PCR, bead RCA, surface RCA, surface SDA, etc., as will be recognized by one of skill in the art. In some embodiments, amplification methods that results in amplification of free DNA molecules in solution or tethered to a suitable matrix by only one end of the DNA molecule can be used. Methods that rely on bridge PCR, where both PCR primers are attached to a surface (see, e.g., WO 2000/018957 and Adessi et al., Nucleic Acids Research (2000): 28(20): E87) can be used. In some cases the methods of the invention can create a “polymerase colony technology”, or “polony”, referring to a multiplex amplification that maintains spatial clustering of identical amplicons (see Harvard Molecular Technology Group and Lipper Center for Computational Genetics website). These include, for example, in situ polonies (Mitra and Church, Nucleic Acid Research 27, e34, Dec. 15, 1999), in situ rolling circle amplification (RCA) (Lizardi et al., Nature Genetics 19, 225, July 1998), bridge PCR (U.S. Pat. No. 5,641,658), picotiter PCR (Leamon et al., Electrophoresis 24, 3769, November 2003), and emulsion PCR (Dressman et al., PNAS 100, 8817, Jul. 22, 2003).

The methods of the present invention may further include a step of hybridizing one or more oligonucleotide primers to an input nucleic acid template. The template can optionally comprise one or more non-canonical nucleotides. In some cases the oligonucleotide primers may comprise a hybridizing portion which comprises random nucleotides, such as for example random dimers, trimers, tetramers, pentamers, hexamers, heptamers, octomers, nonomers, decamers, undecamers, dodecamers, tridecamers, tetradecamers, or longer. In other cases, the hybridizing portion may comprise a non random sequence such as a polyT sequence. In still other cases, the hybridizing portion of some of the oligonucleotide primers may comprise random nucleotides, while the hybridizing portion of some of the nucleotides comprise non-random sequences, such as polyT or “not so random sequences.” In some cases, the hybridizing portion of the oligonucleotide primers may comprise “not so random sequences” such as for example a pool of sequences which randomly or pseudo-randomly prime desired sequences such as total mRNA or a substantial fraction thereof, but do not prime undesired sequences such as rRNA.

A “random primer,” as used herein, can be a primer that generally comprises a sequence that is designed not necessarily based on a particular or specific sequence in a sample, but rather is based on a statistical expectation (or an empirical observation) that the sequence of the random primer is hybridizable (under a given set of conditions) to one or more sequences in the sample. A random primer can generally be an oligonucleotide or a population of oligonucleotides comprising a random sequence(s) in which the nucleotides at a given position on the oligonucleotide can be any of the four nucleotides, or any of a selected group of the four nucleotides (for example only three of the four nucleotides, or only two of the four nucleotides). In some cases all of the positions of the oligonucleotide or population of oligonucleotides can be any of the four nucleotides; in other cases, only a portion of the positions, for instance a particular region, of the oligonucleotide will comprise positions which can be any of the four bases. In some cases, the portion of the oligonucleotide which comprises positions which can be any of the four bases is about 1, 2, 3, 4, 5, 6, 7, 8, 9, 10, 11, 12, 13, 14, or about 15-20 nucleotides in length. In some cases, the portion of the oligonucleotide which comprises positions which can be any of the four bases is about 5-20, 5-15, 5-10, 4-8, 10-20, 15-20, or 10-15 nucleotides in length. In some cases, a random primer may comprise a tailed primer having a 3′-region that comprises a random sequence and a 5′-region that is a non-hybridizing sequence that comprises a specific, non-random sequence. The 3′-region may also comprise a random sequence in combination with a region that comprises poly-T sequences. The sequence of a random primer (or its complement) may or may not be naturally-occurring, or may or may not be present in a pool of sequences in a sample of interest. The amplification of a plurality of RNA species in a single reaction mixture would generally, but not necessarily, employ a multiplicity, or a large multiplicity, of random primers. As is well understood in the art, a “random primer” can also refer to a primer that is a member of a population of primers (a plurality of random primers) which collectively are designed to hybridize to a desired and/or a significant number of target sequences. A random primer may hybridize at a plurality of sites on a nucleic acid sequence. The use of random primers provides a method for generating primer extension products complementary to a target polynucleotide which does not require prior knowledge of the exact sequence of the target. In some embodiments one portion of a primer is random, and another portion of the primer comprises a defined sequence. For example, in some embodiments, a 3′-portion of the primer will comprise a random sequence, while the 5′-portion of the primer comprises a defined sequence. In some embodiments a 3′-random portion of the primer will comprise DNA, and a 5′-portion defined portion of the primer will comprise RNA; in other embodiments, both the 3′ and 5′-portions will comprise DNA. In some embodiments, the 5′-portion will contain a defined sequence and the 3′-portion will comprise a poly-dT sequence that is hybridizable to a multiplicity of RNAs in a sample (such as all mRNA).

The hybridizing portion of the oligonucleotide primers may comprise a pool of hybridizing portions which hybridize to a number of sequences or fragments to be analyzed such as for example, 1; 2; 3; 4; 5; 6; 7; 8; 9; 10; 11; 12; 13; 14; 15; 16; 17; 18; 19; 20; 25; 30; 35; 40; 45; 50; 55; 60; 75; 100; 150; 200; 250; 300; 400; 500; 600; 750; 1000; 10,000; 15,000; 20,000; 25,000; 30,000; 40,000; 50,000; 60,000; 75,000; 100,000; 150,000; 200,000; 250,000 or more sequences or fragments. In some cases, each fragment may be hybridized to one primer, in other cases, each fragment is hybridized on average to 1, 2, 3, 4, 5, 6, 7, 8, 9, 10, 11, 12, 13, 14, 15, 16, 17, 18, 19, 20 or more oligonucleotide primers. Oligonucleotide primers suitable for the methods of the present invention are provided herein.

The olignucleotide primers may be extended along the input nucleic acid template to which they are hybridized. In some cases, the extension may be performed with a polymerase such as for example any of the polymerases provided herein including polymerases comprising strand displacement activity. Exemplary DNA dependent DNA polymerases suitable for the methods of the present invention include but are not limited to Klenow polymerase, with or without 3′-exonuclease, Bst DNA polymerase, Bca polymerase, φ29 DNA polymerase, Vent polymerase, Deep Vent polymerase, Taq polymerase, T4 polymerase, and E. coli DNA polymerase 1, derivatives thereof, or mixture of polymerases. In some cases, the polymerase does not comprise a 5′-exonuclease activity. In other cases, the polymerase comprises 5′ exonuclease activity. In some cases, the primer extension of the present invention may be performed using a polymerase comprising strong strand displacement activity such as for example Bst polymerase. In other cases, the primer extension of the present invention may be performed using a polymerase comprising weak or no strand displacement activity. One skilled in the art may recognize the advantages and disadvantages of the use of strand displacement activity during the primer extension step, and which polymerases may be expected to provide strand displacement activity (see e.g., New England Biolabs Polymerases). For example, strand displacement activity may be useful in ensuring whole genome or whole transcriptome coverage during the random priming and extension step. Strand displacement activity may further be useful in the generation of double stranded amplification products during the priming and extension step. Alternatively, a polymerase which comprises weak or no strand displacement activity may be useful in the generation of single stranded nucleic acid products during primer hybridization and extension that are hybridized to the template nucleic acid.

An “RNA-dependent DNA polymerase” or “reverse transcriptase” (“RT”) is an enzyme that synthesizes a complementary DNA copy from an RNA template. All known reverse transcriptases also have the ability to make a complementary DNA copy from a DNA template; thus, they are both RNA- and DNA-dependent DNA polymerases. Reverse transcriptases may also have an RNase H activity. Some examples of reverse transcriptases are reverse transcriptase derived from Maloney murine leukemia virus (MMLV-RT), avian myeloblastosis virus, retroviral reverse transcriptase, retrotransposon reverse transcriptase, hepatitis B reverse transcriptase, cauliflower mosaic virus reverse transcriptase, bacterial reverse transcriptase, E. coli DNA polymerase and klenow fragment, and Tth DNA polymerase. A primer can be used to initiate synthesis with both RNA and DNA templates. In other examples a DNA dependent DNA polymerase may also comprise an RNA-dependent DNA polymerase such as Klenow polymerase, Bst DNA polymerase and the like.

The extension of hybridized oligonucleotide primers, at least a portion of which may comprise random hybridizing portions, non-random hybridizing portions, not-so random hybridizing portions or a combination thereof, with a polymerase comprising strand displacement activity may provide for the generation of double stranded nucleic acid product fragments. In some cases, the extension of hybridized oligonucleotide primers, at least a portion of which comprise random hybridizing portions, with a polymerase comprising strand displacement activity may produce double stranded nucleic acid products comprising a mixture of double stranded nucleic acid fragment products produced in the polymerization reaction as well as double stranded molecules comprising template nucleic acid hybridized to one or more oligonucleotide primers.

In the embodiment where the template contains one or more non-canonical nucleotides, the products of the primer extension reaction, e.g. single or double stranded, partially double stranded, or mixtures thereof, may be distinguished from the template nucleic acid in that the template nucleic acid comprises one or more non-canonical nucleotides whereas the products of the primer extension reaction do not comprise non-canonical nucleotides, or do not comprise the same one or more non-canonical nucleotides. In some cases, double stranded products of the primer extension reaction comprise a hybrid duplex of a single strand of template nucleic acid comprising one or more non-canonical nucleotides and a single strand of primer extension product that does not comprise one or more non-canonical nucleotides, or does not comprise the same one or more non-canonical nucleotides. In other cases, double stranded products of the primer extension reaction comprise two strands, of which neither strand comprises one or more non-canonical nucleotides, or of which neither strand comprises the same one or more non-canonical nucleotides as the template nucleic acid.

The extension of hybridized oligonucleotide primers may be carried out for a suitable period of time. The period of time for the extension reaction may be anywhere from seconds to minutes to hours. For example, the extension step may include incubation of the input nucleic acid template in a reaction mixture such as the reaction mixtures provided herein with one or more oligonucleotide primers at a temperature suitable for the extension reaction (e.g., 15° C.-80° C.) for a period of between about 5 minutes and about 24 hours. Other suitable extension times include between about 1 minute and about 8 hours, about 2 minutes and about 7 hours, about 3 minutes and about 6 hours, about 4 minutes and about 5 hours, about 5 minutes and about 4 hours, about 5 minutes and about 3 hours, about 5 minutes and about 2 hours, about 10 minutes and about 2 hours, about 15 minutes and about 2 hours, about 20 minutes and about 2 hours, about 30 minutes and about 2 hours, or between about 30 minutes and about 1 hour. Still other suitable extension times include 1 minute, 2 minutes, 3 minutes, 4 minutes, 5 minutes, 6 minutes, 7 minutes, 8 minutes, 9 minutes, 10 minutes, 12 minutes, 15 minutes, 20 minutes, 30 minutes, 45 minutes, 60 minutes, 1 hour, 1.5 hours, 2 hours, 2.5 hours, 3 hours, 3.5 hours, 4 hours or more. Still other suitable extension times include about 1 minute, 2 minutes, 3 minutes, 4 minutes, 5 minutes, 6 minutes, 7 minutes, 8 minutes, 9 minutes, 10 minutes, 12 minutes, 15 minutes, 20 minutes, 30 minutes, 45 minutes, 60 minutes, 1 hour, 1.5 hours, 2 hours, 2.5 hours, 3 hours, 3.5 hours, 4 hours or more.

The extension step may be performed in a reaction mixture comprising nucleotides, labeled nucleotides or a combination thereof. For example, the hybridized oligonucleotides may be extended by one or more polymerases, such as polymerases comprising strand displacement activity or polymerases comprising weak or no strand displacement activity, along the input nucleic acid template in the presence of a mixture of dNTPs and amino allyl dNTPs. The use of amino-allyl dNTPs may allow further labeling and modification of the products of the extension reaction such as double stranded DNA fragment products. For example, the amino allyl dNTPs may provide for biotinylation, fluoresceination, labelling with Cy dyes (e.g., Cy3 or Cy5), or any other nucleic acid modification known in the art. Other modified nucleotides which are suitable for post amplification labeling by either covalent or non-covalent attachment of labels (e.g., fluorophores, chromophores, biotin, antibodies, antigens, or enzymes such as alkaline phosphatase or horse radish peroxidase) are also applicable including for example thio, phosphorothio, and amino modified nucleotides and oligonucleotides as described in U.S. Pat. Nos. 6,172,209, 5,679,785, and 5,623,070, or any other modified nucleotides provided herein.

The methods of the present invention may further include a step of cleaving the input nucleic acid template. In some cases, the input nucleic acid template may be cleaved with an agent such as an enzyme. In the embodiment where the polynucleotide comprises a non-canonical nucleotide, the polynucleotide may be treated with an agent, such as an enzyme, capable of generally, specifically, or selectively cleaving a base portion of the non-canonical deoxyribonucleoside to create an abasic site. As used herein, “abasic site” encompasses any chemical structure remaining following removal of a base portion (including the entire base) with an agent capable of cleaving a base portion of a nucleotide, e.g., by treatment of a non-canonical nucleotide (present in a polynucleotide chain) with an agent (e.g., an enzyme, acidic conditions, or a chemical reagent) capable of effecting cleavage of a base portion of a non-canonical nucleotide. In some embodiments, the agent (such as an enzyme) catalyzes hydrolysis of the bond between the base portion of the non-canonical nucleotide and a sugar in the non-canonical nucleotide to generate an abasic site comprising a hemiacetal ring and lacking the base (interchangeably called “AP” site), though other cleavage products are contemplated for use in the methods of the invention. Suitable agents and reaction conditions for cleavage of base portions of non-canonical nucleotides include: N-glycosylases (also called “DNA glycosylases” or “glycosidases”) including Uracil N-Glycosylase (“UNG”; specifically cleaves dUTP) (interchangeably termed “uracil DNA glycosylase”), hypoxanthine-N-Glycosylase, and hydroxy-methyl cytosine-N-glycosylase; 3-methyladenine DNA glycosylase, 3- or 7-methylguanine DNA glycosylase, hydroxymethyluracile DNA glycosylase; T4 endonuclease V. See, e.g., Lindahl, PNAS (1974) 71(9):3649-3653; Jendrisak, U.S. Pat. No. 6,190,865 B1 or any of the glycosidases provided in Table 1 or homologues thereof such as enzymes with greater than about 50%, 55%, 60%, 65%, 70%, 75%, 80%, 85%, 90%, 95%, 99%, 99.5%, or higher homology or identity at the amino acid or nucleotide level with any of the glycosylases provided herein. In one embodiment, uracil-N-glycosylase is used to cleave a base portion of the non-canonical nucleotide. In other embodiments, the agent that cleaves the base portion of the non-canonical nucleotide is the same agent that cleaves a phosphodiester backbone at the abasic site.

TABLE 1
Glycosylases in bacteria, yeast and humans

| E. | Yeast (S. | | | |
|---|---|---|---|---|
| coli | cerevisiae) | Human | Type | Substrates |
| AlkA | Mag1 | | monofunctional | 3-meA, hypoxanthine |
| UDG | Ung1 | UNG | monofunctional | uracil |
| Fpg | Ogg1 | hOGG1 | bifunctional | 8-oxoG, FapyG |
| Nth | Ntg1 | hNTH1 | bifunctional | Tg, hoU, hoC, urea, |
| | Ntg2 | | | FapyG |
| Nei | | hNEIL1 | bifunctional | Tg, hoU, hoC, urea, |
| | | | | FapyG, FapyA |
| | | hNEIL2 | | AP site, hoU |
| | | hNEIL3 | | unknown |
| MutY | | hMYH | monofunctional | A: 8-oxoG |
| | | hSMUG1 | monofunctional | U, hoU, hmU, fU |
| | | TDG | monofunctional | T: G mispair |
| | | MBD4 | monofunctional | T: G mispair |

Cleavage of base portions of non-canonical nucleotides may provide general, specific or selective cleavage (in the sense that the agent (such as an enzyme) capable of cleaving a base portion of a non-canonical nucleotide generally, specifically or selectively cleaves the base portion of a particular non-canonical nucleotide), whereby substantially all or greater than about 99.9%, 99.5%, 99%, 98.5%, 98%, about 95%, about 90%, about 85%, about 80%, about 75%, about 70%, about 65%, about 60%, about 55%, about 50%, about 45%, or about 40% of the base portions cleaved are base portions of non-canonical nucleotides. However, extent of cleavage can be less. Thus, reference to specific cleavage is exemplary. General, specific or selective cleavage is desirable for control of the fragment size in the methods of generating template polynucleotide fragments of the invention (i.e., the fragments generated by cleavage of the backbone at an abasic site). Reaction conditions may be selected such that the reaction in which the abasic site(s) are created can run to completion, or the reaction may be carried out until 10%, 20%, 30%, 40%, 50%, 60%, 70%, 80%, 90%, 95%, 99%, or about 100% of the non-canonical nucleotides are converted to abasic sites. In some cases, the reaction conditions may be selected such that the reaction in which abasic site(s) are created at between about 10% and about 100% of the one or more non-canonical nucleotides present in the template nucleic acid, between about 20% and about 90%, between about 30% and about 90%, between about 50% and about 90% 95%, 99%, or 100% of the non-canonical nucleotides in the template nucleic acid.

In some embodiments, the template polynucleotide comprising a non-canonical nucleotide is purified following synthesis of the template polynucleotide (to eliminate, for example, residual free non-canonical nucleotides that are present in the reaction mixture). In other embodiments, there is no intermediate purification between the synthesis of the template polynucleotide comprising the non-canonical nucleotide and subsequent steps (such as hybridization of primers, extension of primers to produce primer extension products that do not comprise non-canonical nucleotides, or do not comprise the same non-canonical nucleotides as the template nucleic acid, cleavage of a base portion of the non-canonical nucleotide and cleavage of a phosphodiester backbone at the abasic site).

It is understood that the choice of non-canonical nucleotide can dictate the choice of enzyme to be used to cleave the base portion of that non-canonical enzyme, to the extent that particular non-canonical nucleotides are recognized by particular enzymes that are capable of cleaving a base portion of the non-canonical nucleotide. In some cases, the enzyme is a glycosylase. For example, a template nucleic acid comprising non-canonical nucleotides such as dUTP, 8-oxoguanine, or a methylated purine which may be cleaved by glycosylases may be used in the methods of the present invention. Other suitable non-canonical nucleotides include deoxyinosine triphosphate (dITP), 5-hydroxymethyl deoxycytidine triphosphate (5-OH-Me-dCTP) or any of the non-canonical nucleotides provided in Table 1. See, e.g., Jendrisak, U.S. Pat. No. 6,190,865. A glycosylase such as uracil DNA glycosylase (known as UNG or UDG) which may act on dUTP to provide an abasic site, Ogg1 which may act on 8-oxoguanine to provide an abasic site, or N-methyl purine DNA glycosylase which may act on methylated purines to provide an abasic site may then be used in the methods of the present invention to act on the input nucleic acid template comprising non-canonical nucleotides to initiate a step of cleaving the input nucleic acid template. The enzymes as provided herein may provide N-glycosydic bond cleavage of the input nucleic acid template at the one or more non-canonical nucleotides provided herein to produce one or more abasic (apurinic or apyrimidic) sites.

Additional glycosylases which may be used in the methods of the present invention and their non-canonical nucleotide substrates include 5-methylcytosine DNA glycosylase (5-MCDG), which cleaves the base portion of 5-methylcytosine (5-MeC) from the DNA backbone (Wolffe et al., Proc. Nat. Acad. Sci. USA 96:5894-5896, 1999); 3-methyladenosine-DNA glycosylase I, which cleaves the base portion of 3-methyl adenosine from the DNA backbone (see, e.g. Hollis et al (2000) Mutation Res. 460: 201-210); and/or 3-methyladenosine DNA glycosylase II, which cleaves the base portion of 3-methyladenosine, 7-methylguanine, 7-methyladenosine, and/3-methylguanine from the DNA backbone. See McCarthy et al (1984) EMBO J. 3:545-550. Multifunctional and mono-functional forms of 5-MCDG have been described. See Zhu et al., Proc. Natl. Acad. Sci. USA 98:5031-6, 2001; Zhu et al., Nuc. Acid Res. 28:4157-4165, 2000; and Neddermann et al., J. B. C. 271:12767-74, 1996 (describing bifunctional 5-MCDG; Vairapandi & Duker, Oncogene 13:933-938, 1996; Vairapandi et al., J. Cell. Biochem. 79:249-260, 2000 (describing mono-functional enzyme comprising 5-MCDG activity). In some embodiments, 5-MCDG preferentially cleaves fully methylated polynucleotide sites (e.g., CpG dinucleotides), and in other embodiments, 5-MCDG preferentially cleaves a hemi-methylated polynucleotide. For example, mono-functional human 5-methylcytosine DNA glycosylase cleaves DNA specifically at fully methylated CpG sites, and is relatively inactive on hemimethylated DNA (Vairapandi & Duker, supra; Vairapandi et al., supra). By contrast, chick embryo 5-methylcytosine-DNA glycosylase has greater activity directed to hemimethylated methylation sites. In some embodiments, the activity of 5-MCDG is potentiated (increased or enhanced) with accessory factors, such as recombinant CpG-rich RNA, ATP, RNA helicase enzyme, and proliferating cell nuclear antigen (PCNA). See U.S. Patent Publication No. 20020197639 A1. One or more agents may be used. In some embodiments, the one or more agents cleave a base portion of the same methylated nucleotide. In other embodiments, the one or more agents cleave a base portion of different methylated nucleotides. Treatment with two or more agents may be sequential or simultaneous.

Appropriate reaction media and conditions for carrying out the cleavage of a base portion of a non-canonical nucleotide according to the methods of the invention are those that permit cleavage of a base portion of a non-canonical nucleotide. Such media and conditions are known to persons of skill in the art, and are described in various publications, such as Lindahl, PNAS (1974) 71(9):3649-3653; and Jendrisak, U.S. Pat. No. 6,190,865 B1; U.S. Pat. No. 5,035,996; and U.S. Pat. No. 5,418,149. For example, buffer conditions can be as described above with respect to polynucleotide synthesis. In one embodiment, UDG (Epicentre Technologies, Madison Wis.) is added to a nucleic acid synthesis reaction mixture, and incubated at 37° C. for 20 minutes. In one embodiment, the reaction conditions are the same for the synthesis of a polynucleotide comprising a non-canonical nucleotide and the cleavage of a base portion of the non-canonical nucleotide. In another embodiment, different reaction conditions are used for these reactions. In some embodiments, a chelating regent (e.g. EDTA) is added before or concurrently with UNG in order to prevent a polymerase from extending the ends of the cleavage products.

The polynucleotide comprising an abasic site may be labeled using an agent capable of labeling an abasic site, and, in embodiments involving fragmentation, the phosphodiester backbone of the polynucleotide comprising an abasic site may be cleaved at the site of incorporation of the non-canonical nucleotide (i.e., the abasic site by an agent capable of cleaving the phosphodiester backbone at an abasic site, such that two or more fragments are produced. In embodiments involving fragmentation, labeling can occur before fragmentation, fragmentation can occur before labeling, or fragmentation and labeling can occur simultaneously.

Agents capable of labeling (e.g., generally or specifically labeling) an abasic site, whereby a polynucleotide (or polynucleotide fragment) comprising a labeled abasic site is generated, are provided herein. In some embodiments, the detectable moiety (label) is covalently or non-covalently associated with an abasic site. In some embodiments, the detectable moiety is directly or indirectly associated with an abasic site. In some embodiments, the detectable moiety (label) is directly or indirectly detectable. In some embodiments, the detectable signal is amplified. In some embodiments, the detectable moiety comprises an organic molecule such as a chromophore, a fluorophore, biotin or a derivative thereof. In other embodiments, the detectable moiety comprises a macromolecule such as a nucleic acid, an aptamer, a peptide, or a protein such as an enzyme or an antibody. In other embodiments, the detectable signal is fluorescent. In other embodiments, the detectable signal is enzymatically generated. In some embodiments, the label is selected from, fluorescein, rhodamine, a cyanine dye, an indocyanine dye, Cy3, Cy5, an Alexa Fluor dye, phycoerythrin, 5-(((2-(carbohydrazino)-methyl)thio)acetyl)aminofluorescein, aminooxyacetyl hydrazide (“FARP”), or N-(aminooxyacetyl)-N′-(D-biotinoyl) hydrazine, trifluoroacetic acid salt (ARP).

The cleavage of the input nucleic acid template comprising one or more abasic sites may further be provided by the use of enzymatic or chemical means or by the application of heat, or a combination thereof. For example the input nucleic acid template comprising one or more abasic sites may be treated with a nucleophile or a base. In some cases, the nucleophile is an amine such as a primary amine, a secondary amine, or a tertiary amine. For example, the abasic site may be treated with piperidine, moropholine, or a combination thereof. In some cases, hot piperidine (e.g., 1M at 90° C.) may be used to cleave the input nucleic acid template comprising one or more abasic sites. In some cases, morpholine (e.g., 3M at 37° C. or 65° C.) may be used to cleave the input nucleic acid template comprising one or more abasic sites. Alternatively, a polyamine may be used to cleave the input nucleic acid template comprising one or more abasic sites. Suitable polyamines include for example spermine, spermidine, 1,4-diaminobutane, lysine, the tripeptide K—W—K, N,N-dimethylethylenediamine (DMED), piperazine, 1,2-ethylenediamine, or any combination thereof. In some cases, the input nucleic acid template comprising one or more abasic sites may be treated with a reagent suitable for carrying out a beta elimination reaction, a delta elimination reaction, or a combination thereof. In some cases, the cleavage of input nucleic acid template comprising one or more abasic sites by chemical means may provide fragments of input nucleic acid template, which fragments comprise a blocked 3′ end. In some cases, the blocked 3′ end lacks a terminal hydroxyl. In other cases, the blocked 3′ end is phosphorylated. In still other cases, cleavage of the input nucleic acid template comprising one or more abasic sites by chemical means may provide fragments of input nucleic acid template that are not blocked. In some cases, the methods of the present invention provide for the use of an enzyme or combination of enzymes and a polyamine such as DMED under mild conditions in a single reaction mixture which does not affect the canonical nucleotides and therefore may maintain the sequence integrity of the products of the method. Suitable mild conditions may include conditions at or near neutral pH. Other suitable conditions include pH of about 4.5 or higher, 5 or higher, 5.5 or higher, 6 or higher, 6.5 or higher, 7 or higher, 7.5 or higher, 8 or higher, 8.5 or higher, 9 or higher, 9.5 or higher, 10 or higher, or about 10.5 or higher. Still other suitable conditions include between about 4.5 and 10.5, between about 5 and 10.0, between about 5.5 and 9.5, between about 6 and 9, between about 6.5 and 8.5, between about 6.5 and 8.0, or between about 7 and 8.0. Suitable mild conditions also may include conditions at or near room temperature. Other suitable conditions include a temperature of about 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., or 70° C. or higher. Still other suitable conditions include between about 10° C. and about 70° C., between about 15° C. and about 65° C., between about 20° C. and about 60° C., between about 20° C. and about 55° C., between about 20° C. and about 50° C., between about 20° C. and about 45° C., between about 20° C. and about 40° C., between about 20° C. and about 35° C., or between about 20° C. and about 30° C. In some cases, the use of mild cleavage conditions may provide for less damage to the primer extension products produced by the methods of the present invention. In some cases, the fewer damaged bases, the more suitable the primer extension products may be for downstream analysis such as sequencing, or hybridization. In other cases, the use of mild cleavage conditions may increase final product yields, maintain sequence integrity, or render the methods of the present invention more suitable for automation.

In embodiments involving fragmentation, the backbone of the template polynucleotide comprising the abasic site is cleaved at the abasic site, whereby two or more fragments of the polynucleotide are generated. At least one of the fragments comprises an abasic site, as described herein. Agents that cleave the phosphodiester backbone of a polynucleotide at an abasic site are provided herein. In some embodiments, the agent is an AP endonuclease such as E. coli AP endonuclease IV. In other embodiments, the agent is N,N′-dimethylethylenediamine (termed “DMED”). In other embodiments, the agent is heat, basic condition, acidic conditions, or an alkylating agent. In still other embodiments, the agent that cleaves the phosphodiester backbone at an abasic site is the same agent that cleaves the base portion of a nucleotide to form an abasic site. For example, glycosidases of the present invention may comprise both a glycosidase and a lyase activity, whereby the glycosidase activity cleaves the base portion of a nucleotide (e.g., a non-canonical nucleotide) to form an abasic site and the lyase activity cleaves the phosphodiester backbone at the abasic site so formed. In some cases, the glycosidase comprises both a glycosidase activity and an AP endonuclease activity.

Depending on the agent employed for cleaving at the abasic site of the template polynucleotide, the backbone can be cleaved 5′ to the abasic site (e.g., cleavage between the 5′-phosphate group of the abasic residue and the deoxyribose ring of the adjacent nucleotide, generating a free 3′ hydroxyl group), such that an abasic site is located at the 5′ end of the resulting fragment. In other embodiments, cleavage can also be 3′ to the abasic site (e.g., cleavage between the deoxyribose ring and 3′-phosphate group of the abasic residue and the deoxyribose ring of the adjacent nucleotide, generating a free 5′ phosphate group on the deoxyribose ring of the adjacent nucleotide), such that an abasic site is located at the 3′ end of the resulting fragment. In still other embodiments, more complex forms of cleavage are possible, for example, cleavage such that cleavage of the phosphodiester backbone and cleavage of a portion of the abasic nucleotide results. Selection of the fragmentation agent thus permits control of the orientation of the abasic site within the polynucleotide fragment, for example, at the 3′ end of the resulting fragment or the 5′ end of the resulting fragment. Selection of reaction conditions also permits control of the degree, level or completeness of the fragmentation reactions. In some embodiments, reaction conditions can be selected such that the cleavage reaction is performed in the presence of a large excess of reagents and allowed to run to completion with minimal concern about cleavage of the primer extension products of the invention. By contrast, other methods known in the art, e.g., mechanical shearing, DNase cleavage, cannot distinguish between the template polynucleotide and the primer extension products. In other embodiments, reaction conditions are selected such that fragmentation is not complete (in the sense that the backbone at some abasic sites remains uncleaved (unfragmented)), such that polynucleotide fragments comprising more than one abasic site are generated. Such fragments comprise internal (nonfragmented) abasic sites.

Following generation of an abasic site by cleavage of the base portion of the non-canonical nucleotide if present in the polynucleotide, the backbone of the polynucleotide is cleaved at the site of incorporation of the non-canonical nucleotide (also termed the abasic site, following cleavage of the base portion of the non-canonical nucleotide) with an agent capable of effecting cleavage of the backbone at the abasic site. Cleavage at the backbone (also termed “fragmentation”) results in at least two fragments (depending on the number of abasic sites present in the polynucleotide comprising an abasic site, and the extent of cleavage).

Suitable agents (for example, an enzyme, a chemical and/or reaction conditions such as heat) capable of cleavage of the backbone at an abasic site include: heat treatment and/or chemical treatment (including basic conditions, acidic conditions, alkylating conditions, or amine mediated cleavage of abasic sites, (see e.g., McHugh and Knowland, Nucl. Acids Res. (1995) 23(10):1664-1670; Bioorgan. Med. Chem. (1991) 7:2351; Sugiyama, Chem. Res. Toxicol. (1994) 7: 673-83; Horn, Nucl. Acids. Res., (1988) 16:11559-71), and use of enzymes that catalyze cleavage of polynucleotides at abasic sites, for example AP endonucleases (also called “apurinic, apyrimidinic endonucleases”) (e.g., E. coli Endonuclease IV, available from Epicentre Tech., Inc, Madison Wis.), E. coli endonuclease III or endonuclease IV, E. coli exonuclease III in the presence of calcium ions. See, e.g. Lindahl, PNAS (1974) 71(9):3649-3653; Jendrisak, U.S. Pat. No. 6,190,865 B1; Shida, Nucleic Acids Res. (1996) 24(22):4572-76; Srivastava, J. Biol. Chem. (1998) 273(13):21203-209; Carey, Biochem. (1999) 38:16553-60; Chem Res Toxicol (1994) 7:673-683. As used herein “agent” encompasses reaction conditions such as heat. In one embodiment, the AP endonuclease, E. coli endonuclease IV, is used to cleave the phosphodiester backbone at an abasic site. In another embodiment, cleavage is with an amine, such as N,N′-dimethylethylenediamine. See, e.g., McHugh and Knowland, supra.

Cleavage of the abasic site may occur between the nucleotide immediately 5′ to the abasic residue and the abasic residue, or between the nucleotide immediately 3′ to the abasic residue and the abasic residue (though, as explained herein, 5′ or 3′ cleavage of the phosphodiester backbone may or may not result in retention of the phosphate group 5′ or 3′ to the abasic site, respectively, depending on the fragmentation agent used). Cleavage can be 5′ to the abasic site (such as endonuclease IV treatment which generally results in cleavage of the backbone at a location immediately 5′ to the abasic site between the 5′-phosphate group of the abasic residue and the deoxyribose ring of the adjacent nucleotide, generating a free 3′ hydroxyl group on the adjacent nucleotide), such that an abasic site is located at the 5′ end of the resulting fragment. Cleavage can also be 3′ to the abasic site (e.g., cleavage between the deoxyribose ring and 3′-phosphate group of the abasic residue and the deoxyribose ring of the adjacent nucleotide, generating a free 5′ phosphate group on the deoxyribose ring of the adjacent nucleotide), such that an abasic site is located at the 3′ end of the resulting fragment. Treatment under basic conditions or with amines (such as N,N′-dimethylethylenediamine) results in cleavage of the phosphodiester backbone immediately 3′ to the abasic site. In addition, more complex forms of cleavage are also possible, for example, cleavage such that cleavage of the phosphodiester backbone and cleavage of (a portion of) the abasic nucleotide results. For example, under certain conditions, cleavage using chemical treatment and/or thermal treatment may comprise a β-elimination step which results in cleavage of a bond between the abasic site deoxyribose ring and its 3′ phosphate, generating a reactive α,β-unsaturated aldehyde which can be labeled or can undergo further cleavage and cyclization reactions. See, e.g., Sugiyama, Chem. Res. Toxicol. (1994) 7: 673-83; Horn, Nucl. Acids. Res., (1988) 16:11559-71. It is understood that more than one method of cleavage can be used, including two or more different methods which result in multiple, different types of cleavage products (e.g., fragments comprising an abasic site at the 3′ end, and fragments comprising an abasic site at the 5′ end).

Cleavage of the backbone at an abasic site may be general, specific or selective (in the sense that the agent (such as an enzyme) capable of cleaving the backbone at an abasic site specifically or selectively cleaves the base portion of a particular non-canonical nucleotide), whereby greater than about 98%, about 95%, about 90%, about 85%, or about 80% of the cleavage is at an abasic site. However, extent of cleavage can be less. Thus, reference to specific cleavage is exemplary. General, specific or selective cleavage is desirable for control of the fragment size in the methods of generating labeled polynucleotide fragments of the invention. In some embodiments, reaction conditions can be selected such that the cleavage reaction is performed in the presence of a large excess of reagents and allowed to run to completion with minimal concern about excessive cleavage of the polynucleotide (i.e., while retaining a desired fragment size, which is determined by spacing of the incorporated non-canonical nucleotide, during the synthesis step, above). In other embodiments, extent of cleavage can be less, such that polynucleotide fragments are generated comprising an abasic site at an end and an abasic site(s) within or internal to the polynucleotide fragment (i.e., not at an end).

In embodiments involving cleavage of the phosphodiester backbone, appropriate reaction media and conditions for carrying out the cleavage of the phosphodiester backbone at an abasic site according to the methods of the invention are those that permit cleavage of the phosphodiester backbone at an abasic site. Such media and conditions are known to persons of skill in the art, and are described in various publications, such as Bioorgan. Med. Chem (1991) 7:2351; Sugiyama, Chem. Res. Toxicol. (1994) 7: 673-83; Horn, Nucl. Acids. Res., (1988) 16:11559-71); Lindahl, PNAS (1974) 71(9):3649-3653; Jendrisak, U.S. Pat. No. 6,190,865 B1; Shida, Nucleic Acids Res. (1996) 24(22):4572-76; Srivastava, J. Biol Chem. (1998) 273(13):21203-209; Carey, Biochem. (1999) 38:16553-60; Chem Res Toxicol (1994) 7:673-683.

In some cases, nucleic acids containing abasic sites are heated in a buffer solution containing an amine, for example, 25 mM Tris-HCl and 1-5 mM magnesium ions, for 10-30 minutes at 70° C. to 95° C. Alternatively, 1.0 M piperidine (a base) is added to polynucleotide comprising an abasic site which has been precipitated with ethanol and vacuum dried. The solution is then heated for 30 minutes at 90° C. and lyophilized to remove the piperidine. In another example, cleavage is effected by treatment with basic solution, e.g., 0.2 M sodium hydroxide at 37° C. for 15 minutes. See Nakamura (1998) Cancer Res. 58:222-225. In yet another example, incubation at 37° C. with 100 mM N,N′-dimethylethylenediamine acetate, pH 7.4 is used to cleave. See McHugh and Knowland, (1995) Nucl. Acids Res. 23(10) 1664-1670.

The cleavage of the input nucleic acid template comprising one or more abasic sites may also be performed by enzymatic means. For example an apyrimidinic endonuclease or an apurinic endonuclease (collectively known as AP endonucleases) may be used to cleave the input nucleic acid template at the one or more abasic sites. In some cases, the input nucleic acid template comprising one or more abasic sites may be cleaved with a class I, class II, class III, or class IV AP endonuclease or a combination thereof. In some cases, the cleavage of input nucleic acid template comprising one or more abasic sites by enzymatic means may provide fragments of input nucleic acid template, which fragments comprise a blocked 3′ end. In some cases, the blocked 3′ end lacks a terminal hydroxyl. In other cases, the blocked 3′ end is phosphorylated. In still other cases, cleavage of the input nucleic acid template comprising one or more abasic sites by enzymatic means may provide fragments of input nucleic acid template that are not blocked.

In some cases, the cleavage may be performed by use of a glycosylase and a nucleophile, or a glycosylase and an amine, or a glycosylase and an AP endonuclease such as for example UDG and DMED or UDG and an AP endonuclease at the same time. Alternatively, the input nucleic acid template comprising one or more non-canonical nucleotides may first be treated with a glycosylase to produce one or more abasic sites, and then be treated with an AP endonuclease or cleaved by chemical means. In some cases, the hybridization, and extension reactions are performed first, and then the cleavage reaction is performed after sufficient time. In other cases, the hybridization and extension reactions are performed at the same time as the cleavage reactions. In still other cases, the hybridization and extension reactions are initiated and allowed to proceed for a set period of time (e.g., 1 minute, 2 minutes, 3 minutes, 5 minutes, 10 minutes, 15 minutes, 30 minutes, 1 hour, 2 hours, 3 hours etc.) and then the cleavage reaction is initiated. In some cases, initiation of the cleavage reaction may stop the extension reaction; in other cases, the cleavage reaction and the extension reaction may then proceed concurrently.

For example, E. coli AP endonuclease IV may be added to reaction conditions as described above. AP Endonuclease IV can be added at the same or different time as the agent (such as an enzyme) capable of cleaving the base portion of a non-canonical nucleotide. For example, AP Endonuclease IV can be added at the same time as UNG, or at different times. Alternatively, the template nucleic acid or a reaction mixture comprising template nucleic acid may be treated with UNG and an amine at the same time. A reaction mixture suitable for simultaneous UNG treatment and N,N′-dimethylethylenediamine treatment may include about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, or about 50 mM DMED. Alternatively, the use of an agent that comprises both glycosidase and lyase activity may be utilized in the reaction mixture to cleave the input nucleic acid template.

Cleavage of the input nucleic acid template by chemical means, enzymatic means, or a combination thereof may provide a mixture of double stranded products, single stranded products, and partial duplexes. In some cases, the cleaved products of the cleavage reaction may be removed by one or more methods of the present invention. In some cases, the cleaved products of the cleavage reaction may be removed by purification. For example, the cleaved products of the cleavage reaction may be removed by a size-dependent purification method, or an affinity based purification method. For example, the single stranded nucleic acids may be removed by an affinity hybridization step to capture probes. In some cases, the capture probes may be hybridized to a solid substrate. In other cases, the cleaved nucleic acid products of the cleavage reaction may be removed by an affinity capture step using a ligand with affinity to a label that has been incorporated into the cleaved products of the cleavage reaction. The label, or ligand, may be incorporated prior to cleavage (e.g. during synthesis of the template nucleic acid), during cleavage, or after the cleavage step. In some cases, the label may be incorporated at the abasic site. In other cases, the cleaved nucleic acid products of the cleavage reaction may be removed by a capture step using a reactive moiety (e.g., an amine or a hydrazine) such as an immobilized reactive moiety that reacts with a reactive α,β-unsaturated aldehyde present at the abasic site of the cleaved nucleic acid product of the cleavage reaction. In some cases, the cleaved nucleic acid products of the cleavage reaction may be removed by electrophoresis or ultrafiltration.

In other cases, the single stranded products may be removed by enzymatic means. For example, a single stranded specific exonuclease or endonuclease can be used to cleave the single stranded DNA. A variety of suitable single stranded DNA specific exonucleases are suitable for the methods of the present invention such as for example exonuclease 1, and exonuclease 7. Similarly a variety of suitable single stranded DNA specific endonucleases are suitable for the methods of the present invention such as for example the single stranded DNA specific endonuclease is a S1 endonuclease or a mung bean nuclease. In some cases, any combination of single strand specific endonucleases or exonucleases known in the art such as those provided herein may be utilized to degrade or remove single stranded products, such as single stranded fragmentation products or single stranded primer extension products or a combination thereof.

In some cases, the products of the primer extension reaction generated in the methods of the present invention may be purified from the reaction mixture comprising fragmented target nucleic acid and primer extension products. For example, the primer extension step may include the use of nucleotides comprising a purification label such as for example biotin/avidin or any other suitable label (e.g. digoxin, fluorescein, an antigen, a ligand, a receptor, or any nucleotide labels provided herein). Primer extension products may therefore be understood to contain a member of the biotin/avidin ligand receptor pair or other purification label, whereas primers and template nucleic acid may not. A simple purification step may be performed to remove unincorporated nucleotides such as alcohol or polyethylene glycol precipitation, ion exchange purification, ultrafiltration, silica absorption, or reverse phase methods, and then the primer extension products may be recovered using an appropriate affinity matrix such as a matrix comprising biotin or a derivative thereof, avidin or a derivative thereof, streptavidin or a derivative thereof, an antibody or a derivative or fragment thereof, an antigen, a ligand, or a receptor in the form of particles, beads, a membrane or a column. Alternatively, the simple purification step to remove unincorporated nucleotides may be omitted or performed after the affinity purification step.

In some embodiments, the methods of the present invention further provide for the generation of one or more blunt ended double stranded products. In some embodiments the blunt ended double stranded products are produced from a template not containing any non-canonical nucleotides. In other embodiments the double stranded products are produced from a template containing one or more non-canonical nucleotides. In some cases, the extension step of the present invention directly provides blunt ended double stranded products. In other cases, the extension step of the present invention provides a mixture of blunt ended and non-blunt ended double stranded products. In still other cases, the extension step does not provide blunt ended double stranded products, or does not provide a substantial degree or amount of blunt ended double stranded products. In some cases, the non-blunt ended products of the primer extension reaction must be further treated by the methods of the present invention to produce blunt ended double stranded products, or to convert a substantial fraction of the non-blunt ended products to blunt ended products.

In some cases, the double stranded products generated by the method of the present invention may be blunt ended, when blunt end dsDNA is desirable for downstream analysis such as highly parallel sequencing, or other cloning or adaptor ligation applications, by the use of a single strand specific DNA exonuclease such as for example exonuclease 1, exonuclease 7 or a combination thereof to degrade overhanging single stranded ends of the double stranded products. Alternatively, the double stranded products may be blunt ended by the use of a single stranded specific DNA endonuclease for example but not limited to mung bean endonuclease or S1 endonuclease. Alternatively, the double stranded fragment products may be blunt ended by the use of a polymerase that comprises single stranded exonuclease activity such as for example T4 DNA polymerase, any other polymerase comprising single stranded exonuclease activity or a combination thereof to degrade the overhanging single stranded ends of the double stranded products. In some cases, the polymerase comprising single stranded exonuclease activity may be incubated in a reaction mixture that does or does not comprise one or more dNTPs. In other cases, a combination of single stranded nucleic acid specific exonucleases and one or more polymerases may be used to blunt end the double stranded products of the primer extension reaction. In still other cases, the products of the extension reaction may be made blunt ended by filling in the overhanging single stranded ends of the double stranded products. For example, the fragments may be incubated with a polymerase such as T4 DNA polymerase or Klenow polymerase or a combination thereof in the presence of one or more dNTPs to fill in the single stranded portions of the double stranded products. Alternatively, the double stranded products may be made blunt by a combination of a single stranded overhang degradation reaction using exonucleases and/or polymerases, and a fill-in reaction using one or more polymerases in the presence of one or more dNTPs.

In some embodiments, the methods of the present invention provide for generation of primer extension products comprising double stranded nucleic acids, single stranded nucleic acids, and nucleic acids comprising partial double stranded and partial single stranded portions, either from a template not comprising any non-canonical nucleotides or from a template nucleic acid comprising one or more non-canonical nucleotides; fragmentation of the template nucleic acid; optional purification of the primer extension products; and generation of double stranded products from the single stranded nucleic acid primer extension products and/or from the primer extension products comprising partial double stranded and partial single stranded portions. Methods for generation of double stranded products from partial double stranded products are provided herein including the methods for blunt ending double stranded primer extension products. Methods for generation of double stranded primer extension products from single stranded primer extension products include for example annealing one or more primers, such as any of the primers provided herein, to the single stranded primer extension product and extending the one or more annealed primers with a polymerase, such as any of the polymerases provided herein or a any suitable polymerase in a reaction mixture comprised of one or more dNTPs, including labeled dNTPs, canonical dNTPs, non-canonical dNTPs or a combination thereof. In some cases, the non-canonical nucleotides utilized in the reaction mixture for generating double stranded products from single stranded primer extension products or from partial double stranded products are different from at least one of the non-canonical nucleotides present in the template polynucleotide. Methods of generation of double stranded primer extension products from single stranded primer extension products may further include for example annealing two or more adjacent primers, such as any of the primers provided herein including random primers (e.g. pentamers, hexamers, heptamers, octamers, nonamers, decamers, undecamers, dodecamers, tridecamers etc.), to the single stranded primer extension product and ligating the adjacent primers. Methods for generating double stranded primer extension products from single stranded primer extension products may further include for example annealing one or more primers such as any of the primers provided herein including primers comprising random hybridizing portions (e.g. random pentamers, hexamers, heptamers, octamers, nonamers, decamers, undecamers, dodecamers, tridecamers etc.) to the single stranded primer extension product and extending the annealed primers. In some cases, the extension step may be performed using an enzyme (e.g., a DNA dependent DNA polymerase) comprising strand displacement activity.

In some embodiments, the methods of the present invention provide for attachment (e.g., ligation) of adaptor molecules to the double stranded DNA products of the primer extension reaction, or double stranded products generated from the single stranded or partially double stranded products of the primer extension reaction. The adaptor molecules may be ligated to double stranded DNA fragment molecules comprising single stranded overhangs, including but not limited to single, double, triple, quadruple, quintuple, sextuple, septuple, octuple, or more base overhangs, or to double stranded DNA fragment molecules comprising blunt ends. In some cases, the adaptor molecules are ligated to blunt end double stranded DNA fragment molecules which have been modified by 5′ phosphorylation. In some cases, the adaptor molecules are ligated to blunt end double stranded DNA fragment molecules which have been modified by 5′ phosphorylation followed by extension of the 3′ end with one or more nucleotides. In some cases, the adaptor molecules are ligated to blunt end double stranded DNA fragment molecules which have been modified by 5′ phosphorylation followed by extension of the 3′ end with a single nucleotide (or 2, 3, 4, 5, 6, 7, 8, 9, 10, 11, 12, 13, 14, 15, 16, 17, 18, 19, 20 or more) such as for example adenine, guanine, cytosine, or thymine. In still other cases, adaptor molecules can be ligated to blunt end double stranded DNA fragment molecules which have been modified by extension of the 3′ end with one or more nucleotides followed by 5′ phosphorylation. In some cases, extension of the 3′ end may be performed with a polymerase such as for example Klenow polymerase or any of the suitable polymerases provided herein, or by use of a terminal deoxynucleotide transferase, in the presence of one or more dNTPs in a suitable buffer containing magnesium. Phosphorylation of 5′ ends of DNA fragment molecules may be performed for example with T4 polynucleotide kinase in a suitable buffer containing ATP and magnesium.

The adaptor molecules may comprise single or double stranded nucleic acids or a combination thereof. In some cases, the adaptor molecules comprise a one, two, three, four, five, six, seven, eight, nine, ten, eleven, twelve, thirteen, fourteen, fourteen, fifteen, sixteen, seventeen, eighteen, nineteen, twenty or longer base long single stranded overhang at their 5′ ends. For example, the adaptor molecules may comprise a one base long thymine, adenine, cytosine, or guanine overhang at their 5′ ends. Adaptor molecule compositions are provided herein.

In some embodiments, the methods of the present invention provide for ligation or attachment of adaptor molecules to the single stranded DNA products of the extension reaction. The adaptor molecules may comprise single stranded or double stranded nucleic acids or a combination thereof. The adaptor molecules may be ligated to the single stranded. DNA products of the extension reaction using T4 RNA ligase which is capable of ligating two single stranded nucleic acids (RNA or DNA) together in the absence of a template. Alternatively, a single stranded DNA specific ligase such as for example CircLigase® may be utilized in the methods of the present invention.

In some embodiments, the methods of the present invention provide for contacting an input nucleic acid template comprising one or more non-canonical nucleotides with a reaction mixture. In some cases, the reaction mixture may comprise one or more oligonucleotide primers as provided herein. For example, the reaction mixture may comprise one or more oligonucleotide primers comprising random hybridizing portions. Additionally, the reaction mixture may comprise one or more oligonucleotide primers comprising random hybridizing portions and one or more oligonucleotide primers comprising a polyT sequence.

In some cases, the reaction mixture may comprise one or more polymerases as provided herein. For example, the reaction mixture may comprise one or more polymerases comprising strand displacement activity, such as for example, Klenow polymerase, exo-Klenow polymerase, 5′-3′ exo-Klenow polymerase, Bst polymerase, Bst large fragment polymerase, Vent polymerase, Deep Vent (exo-) polymerase, 9° Nm polymerase, Therminator polymerase, Therminator II polymerase, MMulV Reverse Transcriptase, phi29 polymerase, or DyNAzyme EXT polymerase, or a combination thereof. In some cases, the reaction mixture may be configured to provide double stranded products in the presence of the input nucleic acid template, the one or more oligonucleotide primers, and the one or more polymerases comprising strand displacement activity. Enzymes for use in the compositions, methods and kits of the present invention may further include any enzyme having reverse transcriptase activity. Such enzymes include, but are not limited to, retroviral reverse transcriptase, retrotransposon reverse transcriptase, hepatitis B reverse transcriptase, cauliflower mosaic virus reverse transcriptase, bacterial reverse transcriptase, E. coli DNA polymerase and klenow fragment, Tth DNA polymerase, Taq DNA polymerase (Saiki, R. K., et al., Science 239:487-491 (1988); U.S. Pat. Nos. 4,889,818 and 4,965,188), Tne DNA polymerase (WO 96/10640), Tma DNA polymerase (U.S. Pat. No. 5,374,553), C. Therm DNA polymerase from Carboxydothermus hydrogenoformans (EP0921196A1, Roche, Pleasanton, Calif., Cat. No. 2016338), ThermoScript (Invitrogen, Carlsbad, Calif. Cat. No. 11731-015) and mutants, fragments, variants or derivatives thereof. As will be understood by one of ordinary skill in the art, modified reverse transcriptases may be obtained by recombinant or genetic engineering techniques that are routine and well-known in the art. Mutant reverse transcriptases can, for example, be obtained by mutating the gene or genes encoding the reverse transcriptase of interest by site-directed or random mutagenesis. Such mutations may include point mutations, deletion mutations and insertional mutations. Preferably, one or more point mutations (e.g., substitution of one or more amino acids with one or more different amino acids) are used to construct mutant reverse transcriptases of the invention. Fragments of reverse transcriptases may be obtained by deletion mutation by recombinant techniques that are routine and well-known in the art, or by enzymatic digestion of the reverse transcriptase(s) of interest using any of a number of well-known proteolytic enzymes. Mutant DNA polymerase containing reverse transcriptase activity can also be used as described in U.S. patent application Ser. No. 10/435,766, incorporated herein by reference.

In some cases, the reaction mixture may comprise one or more agents capable of cleaving the base portion of a non-canonical nucleotide to generate an abasic site. In some cases, the reaction mixture may contain the one or more agents capable of cleaving the base portion of a non-canonical nucleotide to generate an abasic site at the initiation of the extension reaction. In some cases, the reaction mixture may be supplemented with the one or more agents capable of cleaving the base portion of a non-canonical nucleotide to generate an abasic site after a suitable period of time (e.g., about 1, 2, 3, 4, 5, 10, 15, 20, 25, 30, 45, 60, 90, 120, 180, 240, 300, 400, 500, 600 minutes) has passed for the generation of primer extension products. Suitable agents capable of cleaving the base portion of a non-canonical nucleotide to generate an abasic site include but are not limited to UDG and MPG.

In some cases, the reaction mixture may comprise one or more agents capable of fragmenting a phosphodiester backbone at an abasic site to fragment the input nucleic acid template. In some cases, the reaction mixture may contain the one or more agents capable of fragmenting a phosphodiester backbone at an abasic site to fragment the input nucleic acid template at the initiation of the extension reaction. In some cases, the reaction mixture may be supplemented with the one or more agents capable of fragmenting a phosphodiester backbone at an abasic site to fragment the input nucleic acid template after a suitable period of time (e.g., about 1, 2, 3, 4, 5, 10, 15, 20, 25, 30, 45, 60, 90, 120, 180, 240, 300, 400, 500, 600 minutes) has passed for the generation of primer extension products. Suitable agents capable of fragmenting a phosphodiester backbone at an abasic site to fragment the input nucleic acid template include but are not limited to an amine, a primary amine, a secondary amine, a polyamine as provided herein, a nucleophile, a base (e.g. NaOH), piperidine, hot piperidine, and one or more AP endonucleases.

The methods of the present invention provide for downstream analysis of the primer extension products generated in the methods of the present invention. Said downstream analysis includes but is not limited to e.g. pyrosequencing, sequencing by synthesis, sequencing by hybridization, single molecule sequencing, nanopore sequencing, and sequencing by ligation, high density PCR, microarray hybridization, SAGE, digital PCR, and massively parallel Q-PCR; subtractive hybridization; differential amplification; comparative genomic hybridization, preparation of libraries (including cDNA and differential expression libraries); preparation of an immobilized nucleic acid (which can be a nucleic acid immobilized on a microarray), and characterizing amplified nucleic acid products generated by the methods of the invention, or a combination thereof.

Large scale sequencing methods provided by the present invention include methods such as those provided by the Genome Analyzer by Illumina, the Genome Sequencer by 454/Roche Lifesciences, the SMRT platform by Pacific Biosciences, the HeliScope by Helicos Biosciences, the Polonator by Dover Systems, and semiconductor sequencing (Personal Genome Machine (PGM™) by Ion Torrent.

In some embodiments, the methods are useful for preparing target polynucleotide(s) for sequencing by synthesis using the methods commercialized by 454/Roche Lifesciences including but not limited to the methods and apparatus described in Margulies et al., Nature (2005) 437:376-380 (2005); and U.S. Pat. Nos. 7,244,559; 7,335,762; 7,211,390; 7,244,567; 7,264,929; 7,323,305. In general, polynucleotides may be prepared by primer extension from a template comprising non-canonical nucleotides according to the methods of the present invention, which methods may further include cleavage of the template nucleic acid, purification of the primer extension products, adaptor ligation, further amplification, or a combination thereof. The primer extension products may then be immobilized onto beads, and compartmentalized in a water-in-oil emulsion suitable for amplification by PCR. In some cases, alternative amplification methods other than PCR may be employed in the water-in-oil emulsion such as any of the methods provided herein. When the emulsion is broken, amplified fragments remain bound to the beads. The methods may include a step of rendering the nucleic acid bound to the beads single stranded or partially single stranded. The beads may be enriched and loaded, into wells of a fiber optic slide so that there is approximately 1 bead in each well. Nucleotides are flowed across and into the wells in a fixed order in the presence of polymerase, sulfhydrolase, and luciferase. Addition of nucleotides complementary to the target strand results in a chemiluminescent signal that is recorded such as by a camera. The combination of signal intensity and positional information generated across the plate allows software to determine the DNA sequence.

In some embodiments, the methods are useful for preparing target polynucleotide(s) for sequencing by the methods commercialized by Helicos BioSciences Corporation (Cambridge, Mass.) as described in U.S. patent application Ser. No. 11/167,046, and U.S. Pat. Nos. 7,501,245; 7,491,498; 7,276,720; and in U.S. Patent Application Publication Nos. US20090061439; US20080087826; US20060286566; US20060024711; US20060024678; US20080213770; and US20080103058. In general, polynucleotides may be prepared by primer extension from a template comprising non-canonical nucleotides according to the methods of the present invention, which methods may further include cleavage of template nucleic acid, purification of the primer extension products, adaptor ligation, further amplification, or a combination thereof. The primer extension products may then be immobilized onto a flow-cell surface. The methods may include a step of rendering the nucleic acid bound to the flow-cell surface stranded or partially single stranded. Polymerase and labeled nucleotides are then flowed over the immobilized DNA. After fluorescently labeled nucleotides are incorporated into the DNA strands by a DNA polymerase, the surface is illuminated with a laser, and an image is captured and processed to record single molecule incorporation events to produce sequence data.

In some embodiments, the methods are useful for preparing target polynucleotide(s) for sequencing by the sequencing by ligation methods commercialized by Applied Biosystems (e.g., SOLiD sequencing). In general, polynucleotides may be prepared by primer extension from a template comprising non-canonical nucleotides according to the methods of the present invention, which methods may further include cleavage of template nucleic acid, purification of the primer extension products, adaptor ligation, further amplification, or a combination thereof. The primer extension products may then be incorporated into a water in oil emulsion along with polystyrene beads and amplified by for example PCR. In some cases, alternative amplification methods may be employed in the water-in-oil emulsion such as any of the methods provided herein. The amplified product in each water microdroplet formed by the emulsion interact, bind, or hybridize with the one or more beads present in that microdroplet leading to beads with a plurality of amplified products of substantially one sequence. When the emulsion is broken, the beads float to the top of the sample and are placed onto an array. The methods may include a step of rendering the nucleic acid bound to the beads stranded or partially single stranded. Sequencing primers are then added along with a mixture of four different fluorescently labeled oligonucleotide probes. The probes bind specifically to the two bases in the polynucleotide to be sequenced immediately adjacent and 3′ of the sequencing primer to determine which of the four bases are at those positions. After washing and reading the fluorescence signal form the first incorporated probe, a ligase is added. The ligase cleaves the oligonucleotide probe between the fifth and sixth bases, removing the fluorescent dye from the polynucleotide to be sequenced. The whole process is repeated using a different sequence primer, until all of the intervening positions in the sequence are imaged. The process allows the simultaneous reading of millions of DNA fragments in a ‘massively parallel’ manner. This ‘sequence-by-ligation’ technique uses probes that encode for two bases rather than just one allowing error recognition by signal mismatching, leading to increased base determination accuracy.

In some embodiments, the methods are useful for preparing target polynucleotide(s) for sequencing by the sequencing by ligation methods commercialized by Dover Systems. In general, polynucleotides may be prepared by primer extension from a template comprising non-canonical nucleotides according to the methods of the present invention, which methods may further include cleavage of template nucleic acid, purification of the primer extension products, adaptor ligation, further amplification, or a combination thereof. The polynucleotides may then amplified in an emulsion in the presence of magnetic beads. Any amplification methods may be employed in the water-in-oil emulsion such as any of the methods provided herein. The resulting beads with immobilized clonal polynucleotide polonies are then purified by magnetic separation, capped, amine functionalized, and covalently immobilized in a series of flow cells. The methods may include a step of rendering the nucleic acid bound to the flow-cell surface stranded or partially single stranded. Then, a series of anchor primers are flowed through the cells, where they hybridize to the synthetic oligonucleotide sequences at the 3′ or 5′ end of proximal or distal genomic DNA tags. Once an anchor primer is hybridized, a mixture of fully degenerate nonanucleotides ('nonamers') and T4 DNA ligase is flowed into the cell; each of the nonamer mixture's four components being labeled with one of four fluorophores, which correspond to the base type at the query position. The fluorophore-tagged nonamers selectively ligate onto the anchor primer, providing a fluorescent signal that identifies the corresponding base on the genomic DNA tag. Once the probes are ligated, fluorescently labeling the beads, the array is imaged in four colors. Each bead on the array will fluoresce in only one of the four images, indicating whether there is an A, C, G, or T at the position being queried. After imaging, the array of annealed primer-fluorescent probe complex, as well as residual enzyme, are chemically striped using guanidine HCl and sodium hydroxide. After each cycle of base reads at a given position have been completed, and the primer-fluorescent probe complex has been stripped, the anchor primer is replaced, and a new mixture of fluorescently tagged nonamers is introduced, for which the query position is shifted one base further into the genomic DNA tag. Seven bases are queried in this fashion, with the sequence performed from the 5′ end of the proximal tag, followed by six base reads with a different anchor primer from the 3′ end of the proximal tag, for a total of 13 base pair reads for this tag. This sequence is then repeated for the 5′ and 3′ ends of the distal tag, resulting in another 13 base pair reads. The ultimate result is a read length of 26 bases (thirteen from each of the paired tags). However, it is understood that this method is not limited to 26 base read lengths.

In some embodiments, the methods are useful for preparing target polynucleotide(s) for sequencing by the methods commercialized by Illumina as described U.S. Pat. Nos. 5,750,341; 6,306,597; and 5,969,119. In general, polynucleotides may be prepared by primer extension from a template comprising non-canonical nucleotides according to the methods of the present invention, which methods may further include cleavage of template nucleic acid, purification of the primer extension products, adaptor ligation, further amplification, or a combination thereof. The methods of the present invention may provide amplified nucleic acid sequences tagged at one (e.g. (A)/(A′) or both ends (e.g. (A)/(A′) and (C)/(C′)). In some cases, single stranded nucleic acid tagged at one or both ends is amplified by the methods of the present invention (e.g. by SPIA or linear PCR). The resulting nucleic acid is then denatured and the single stranded amplified polynucleotides are randomly attached to the inside surface of flow-cell channels. Unlabeled nucleotides are added to initiate solid-phase bridge amplification to produce dense clusters of double-stranded DNA. The initiate the first base sequencing cycle, four labeled reversible terminators, primers, and DNA polymerase are added. After laser excitation, florescence form each cluster on the flow cell is imaged. The identity of the first base for each cluster is then recorded. Cycles of sequencing are performed to determine the fragment sequence one base at a time. For paired-end sequencing, such as for example, when the polynucleotides are labeled at both ends by the methods of the present invention, sequencing templates can be regenerated in-situ so that the opposite end of the fragment can also be sequenced.

In some embodiments, the methods are useful for preparing target polynucleotide(s) for sequencing by the methods commercialized by Pacific Biosciences as described in U.S. Pat. Nos. 7,462,452; 7,476,504; 7,405,281; 7,170,050; 7,462,468; 7,476,503; 7,315,019; 7,302,146; 7,313,308; and U.S. Patent Application Publication Nos. US20090029385; US20090068655; US20090024331; and US20080206764. In general, polynucleotides may be prepared by primer extension from a template comprising non-canonical nucleotides according to the methods of the present invention, which methods may further include cleavage of template nucleic acid, purification of the primer extension products, adaptor ligation, further amplification, or a combination thereof. The polynucleotides may then be immobilized in zero mode waveguide arrays. The methods may include a step of rendering the nucleic acid bound to the waveguide arrays single stranded or partially single stranded. Polymerase and labeled nucleotides are added in a reaction mixture, and nucleotide incorporations are visualized via fluorescent labels attached to the terminal phosphate groups of the nucleotides. The fluorescent labels are clipped off as part of the nucleotide incorporation. In some cases, circular templates are utilized to enable multiple reads on a single molecule.

In some embodiments, the methods of the present invention are useful for preparing target polynucleotide(s) for sequencing by the methods commercialized by Ion Torrent Systems. Such methods are described, for example, in U.S. Patent Application Publication Nos. 20100197507, 20100188073, 20100137143, 20100035252, 20090127589, and 20090026082. Ion Torrent Systems technology can use chemical-sensitive field effect transistors (FETs). Ion Torrent Systems technology can include use of a semiconductor chip that comprises multiple layers, e.g., a layer with micro-machined wells, an ion-sensitive layer, and an ion sensor layer. Nucleic acids attached to beads can be introduced into the micro-machined wells. A clonal population of single nucleic acids can be attached to a single bead. One type of deoxyribonucleotide (e.g., dATP, dCTP, dGTP, or dTTP) can be introduced into the micro-machined wells to initiate sequencing of the nucleic acids on the beads. Upon incorporation of nucleotides by DNA polymerase, protons are released in the well which can be detected by the ion sensor. The semiconductor chip can then be washed and the process can be repeated with a different deoxyribonucleotide. A plurality of nucleic acids can be sequenced in the micro-machined wells of a semiconductor chip.

In some embodiments, the methods of the present invention are useful for preparing target polynucleotide(s) for analysis by array comparative genomic hybridization as described for example in U.S. Pat. Nos. 5,856,097, 5,965,362, 5,976,790, 5,665,549, 6,335,167, 6,159,685, 7,238,484, 7,534,567, 7,537,895; and U.S. Patent Application Publication Nos. US20050118634, US20050260645, US20050282227, US20060292608, US20070087355, US20070134676, US20070160988, US20070219727, and US20090069191. In general, polynucleotides may be prepared by primer extension from a template comprising non-canonical nucleotides according to the methods of the present invention, which methods may further include cleavage of template nucleic acid, purification of the primer extension products, adaptor ligation, further amplification, or a combination thereof. Specific cleavage of the template nucleic acid and optional purification of the primer extension products from the cleaved template nucleic acid may provide for elimination or reduction of potential cross-hybridization of array probes with non-labeled input template, as well as increased specific activity of the labeled targets for hybridization to array probes. The primer extension products may further be labeled by any methods known in the art including methods provided herein including by incorporation of labeled nucleotides by a polymerase during primer extension, during further rounds of amplification of the primer extension products, or by end labeling. In some cases, incorporation of labeled nucleotides by a polymerase during primer extension may be performed by random priming of template nucleic acid comprising one or more non-canonical nucleotides, not so random priming of template nucleic acid comprising one or more non-canonical nucleotides, or non random priming of template nucleic acid comprising one or more non-canonical nucleotides. In some cases, incorporation of labeled nucleotides may be provided by using primers which are labeled. In still other cases, incorporation of labeled nucleotides may be provided by performing further rounds of amplification such as any of the amplification means provided herein in the presence of one or more labeled nucleotides or labeled primers. End labeling reactions may be performed using a T4 RNA ligase in the presence of a suitable label such as a biotinylated or fluorophore conjugated nucleotide. Alternatively, end-labeling reactions may be performed with a terminal deoxynucleotide transferase in the presence of a suitable label such as biotinylated ddUTP or a fluorophore conjugated ddUTP.

In some cases, primer extension products prepared from different biological samples such as samples from two different organisms or samples from suspected or known normal tissue and samples from suspected or known disease or tumor tissue may be differentially labeled. For example, nucleic acid primer extension products provided from samples comprising normal tissue may be labeled with one detectable identifying characteristic (e.g. fluorophore, chromophore, biotin, avidin, antigen, antibody, protein, enzyme, alkaline phosphatase or horse radish peroxidase), while primer extension products from samples comprising disease or tumor tissue may be labeled with a different detectable identifying characteristic. In some cases, the differentially labeled primer extension products represent a control set of primer extension products and a test set of primer extension products. In some cases, after differential labeling of the primer extension products of the present invention, the products may be mixed and contacted with an array of positionally addressable immobilized oligonucleotide probes. The degree of hybridization may be measured by standard methods known in the art such as fluorescence detection, and the data may be normalized, and analyzed to detect differences in the nucleic acids such as differences in copy number of nucleic acid sequences, or the presence or absence of SNPs or other polymorphisms such as variable nucleotide repeats, inserts, and deletions.

In the methods of the invention, the steps may be carried out in the order listed or, in some cases, may be carried out in a different order. In some methods a later step depends on the formation of a product from an earlier step, in which case such steps must be carried out in the order listed. One of ordinary skill in the art will understand which steps should be carried out in the order listed, and which steps can be carried out in a different order.

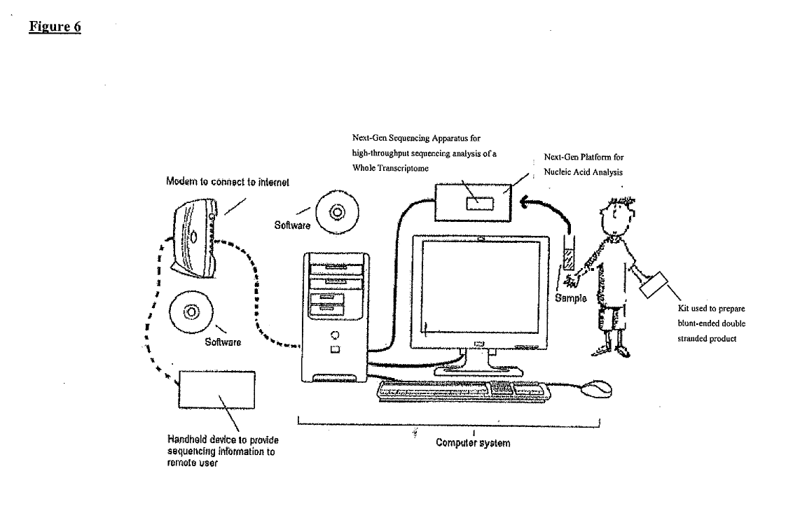

In the methods of the invention described herein, the analysis of the nucleic acid products generated may be automated such that the downstream reactions are performed via robotics. Once performed, data obtained may be provided to a personal computer, a personal digital assistant, a cellular phone, a video game system, or a television so that a user can monitor the progress of the sequencing reactions remotely. This process is illustrated, for example, in FIG. 6. The performing, monitoring and obtaining of results of the downstream reactions can be done from a place other than where the apparatus is located, for example from a physically separate room, building, city, state, country or the like. Likewise, the results can be transmitted to the remote user from a physically separate room, building, city, state, country or the like.

In some embodiments, the components provided herein are added simultaneously at the initiation of the amplification process. In another embodiment, components are added in any order prior to or after appropriate time points during the amplification process, as required and/or permitted by the amplification reaction. Such time points, some of which are noted below, can be readily identified by a person of skill in the art. The enzymes used for nucleic acid amplification according to the methods of the invention can be added to the reaction mixture either prior to a target nucleic acid denaturation step, following a denaturation step, or following hybridization of the primer to the target nucleic acid, as determined by their thermal stability and/or other considerations known to the person of skill in the art. The oligonucleotide primer extension reactions can be performed consecutively or simultaneously with the other method steps of the present invention. In these embodiments, the reaction conditions and components may be varied between the different reactions.

In some embodiments, the methods can be stopped at various time points, and resumed at a later time. Said time points can be readily identified by a person of skill in the art. One time point is at the end of the oligonucleotide primer extension reaction. Another time point is at the end of the cleavage reaction. Another time point is at the end of any purification step. Methods for stopping the reactions include for example, cooling the reaction mixture to a temperature that inhibits enzyme activity, heating the reaction mixture to a temperature that destroys an enzyme, or purifying the desired nucleic acid or fragment thereof from the reaction mixture. Methods for resuming the reactions include, for example, raising the temperature of the reaction mixture to a temperature that permits enzyme activity, replenishing a destroyed (depleted) enzyme, or contacting the nucleic acid with a suitable reaction mixture. In some embodiments, one or more of the components of the reactions is replenished prior to, at, or following the resumption of the reactions. Alternatively, the reaction can be allowed to proceed (i.e., from start to finish) without interruption.

In some embodiments the reaction can be allowed to proceed without purification of intermediate complexes, for example, to remove primer, or single stranded cleavage fragments. Products can be purified at various time points, which can be readily identified by a person of skill in the art. One time point is at the end of the primer extension reaction. Another time point is at the end of input nucleic acid template cleavage. Yet another time point is at the end of ligation of adaptor molecules to the double stranded DNA fragments. In some embodiments, the removal of primers and/or template at the end of a defined step by enzymes with appropriate nuclease activities are also useful, for example, cleavage of the input nucleic acid template comprising one or more non-canonical nucleotides with a glycosylase and an amine.

Strand Specific (Strand Tracking) RNA Amplification and Direct cDNA Sequencing (RNA-Seq)

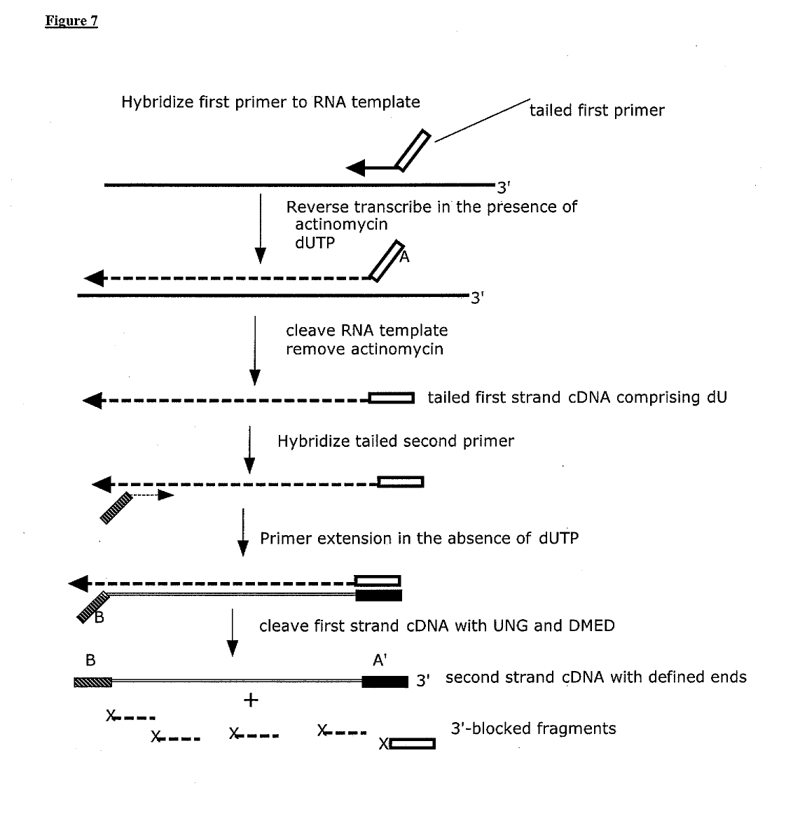

One embodiment of a method for the generation of second strand cDNA with defined sequences at 3′- and 5′-ends is shown in FIG. 7 (sequence A′ at the 3′-end and sequence B at the 5′-end).

In FIG. 7, sequence A is the 5′-tail sequence of a first tailed primer, employed for the generation of first strand cDNA. The first primer comprises a 3′-sequence that can hybridize to an input RNA target (template) and a 5 sequence A. The tail sequence A can be DNA.

Reverse transcription of the input RNA target (template) can be carried out by extension of a first primer or primers hybridized to the RNA target by an RNA-dependent DNA polymerase. The reaction can be carried out in the presence of actinomycin D, an inhibitor of the DNA-dependent DNA polymerase activity of the reverse transcriptase. The presence of actinomycin D can be used to establish extension of the hybridized primer to RNA targets only and prevent extension along a DNA target which may be in the sample comprising the RNA target (template). In addition, presence of actinomycin D can prevent primer extension along the first primer extension product, or products, to generate second strand synthesis in the reverse transcriptase first strand synthesis, thus establishing the strand specificity of the primer extension products generated in the reverse transcription reaction. There are several known DNA-dependent DNA polymerase inhibitors known in the art that can be used in the methods of the provided invention. For example, Actinomycin-D acts as a DNA-dependent DNA polymerase inhibitor by binding to DNA and preventing initiation of replication (Guy and Taylor (1978) PNAS 75:6088-92.) Other examples of DNA-dependent DNA polymerase inhibitors include, but are not limited to, actinomycin (dactinomycin), alpha-amanitin, aphidicolin (Cozad and Warner (1982) Gamete Research 6:155-60; Gonzcol and Plotkin (1985) Arch Virology 84:129-34; Haraguchi et al. (1983) Nucleic Acids Research 11:1197-1209), BP5, novobiocin (Schneck and Staudenbauer (1977) Nuc Acids Res 4:2057-64), rifampicin, rifamycin (Frolova et al. (1977) Nuc Acids Res 4:523-8), sulfoquinovosylmonoacylglycerol, sulfoquinovosyldiacylglycerol (Ohta et al. (2000) Mutat Res 467:139-52; Ohta et al. (1999) Biol Pharm Bull 22:111-16), ursane, oleanane triterpenoids, ursolic acid, oleanolic acid (Deng et al. (1999) J Nat Prod 62:1624-6), mikanolide, dihydromikanolide (U.S. Pat. No. 6,767,561), dehydroaltenusin (Mizushina et al. (2000) J Biol Chem 275:33957-61), catapol (Pungitore et al. (2004) J Nat Prod 67:357-61), taxinine, cephalomanninine (Oshige et al. (2004) Bioorganic and Medicinal Chem 12:2597-601), dipeptide alcohols (Kato et al. (2005) Int J Mol Med 16:653-9), corylifolin; bakuchiol; resveratrol; Neobavaisoflavone; daidzein; bakuchicin (Sun et al. (1998) J Nat Prod 61:362-6), levodopa, dopamine (Wick (1980) Cancer Research 40:1414-8), anacardic acid and oleic acid (Chen et al. (1998) Chem Comm 24:2769-70).

The DNA-dependent DNA polymerase inhibitors of the present invention can be added in an amount effective to inhibit DNA-dependent DNA polymerase activity. As a result, replication of DNA, when present in the sample in combination with RNA target or targets, is inhibited. The amount of a DNA-dependent DNA polymerase inhibitor that should be added to achieve the desired inhibition is well known and understood in the art. For example, the DNA-dependent DNA polymerase inhibitor Actinomycin D may be added in a concentration from about 0.1 μg/ml to about 100 μg/ml. In a preferred embodiment, 50% of DNA replication is inhibited by addition of a DNA-dependent DNA polymerase inhibitor. In another preferred embodiment, 60% of DNA replication is inhibited by addition of a DNA-dependent DNA polymerase inhibitor. In yet other preferred embodiments, 70%, 80%, 85%, 90%, 95%, 96%, 97%, 98%, 99% or 100% of DNA replication is inhibited by addition of a DNA-dependent DNA polymerase inhibitor.

Combinations of DNA-dependent DNA polymerase inhibitors may be used to achieve the desired inhibition. Use of combinations of DNA-dependent DNA polymerase inhibitors may be desirable to minimize unwanted effects on the reaction conditions by decreasing the concentration of each inhibitor in the reaction mixture. Alternatively, the combination of inhibitors may be able to achieve a greater amount of inhibition, such as by inhibiting DNA-dependent DNA polymerase activity at various stages.

A first strand cDNA synthesis reaction mixture can comprise a non-canonical nucleotide. For example, a first strand cDNA synthesis reaction mixture can further comprise dUTP and the four canonical dNTPs to generate primer extension products comprising dU. Other non canonical nucleotide triphosphates described herein can similarly be employed to generate tailed first strand synthesis comprising non canonical nucleotides.

Second strand synthesis can be carried out following cleavage of the RNA targets and a clean-up step which can remove the actinomycin D from the reaction products. RNA in the complex with first strand cDNA can be cleaved, for example, with an RNase which cleaves RNA in the RNA-DNA heteroduplex. RNA can also be cleaved by heating the reaction mixture, for example, heating the reaction mixture at 75° C. for 5 to 15 minutes.

Second strand synthesis can be carried out following hybridization of a second tailed primer to the first strand cDNA generated as described above. The second primer can comprise a 3′-sequence that can hybridize to the first cDNA comprising dU and a 5′-tail A. The second primer can further comprise a 5′-tail sequence B which is not complementary to the first strand cDNA. Primer extension along the first strand cDNA template can be carried out by DNA polymerase to produce a double stranded cDNA comprising a complex of the first and second primer extension products. The second strand cDNA synthesis can be carried out in the absence of the non canonical nucleotide tri phosphate (dUTP, for example) and can comprise a complement of the 5′-tail sequence of the first primer, sequence A′, at the 3′-end.

Cleavage of the first strand cDNA in the complex can be achieved by combining the reaction mixture with an enzyme that cleaves the base portion of the non canonical nucleotide to generate an abasic site, and an amine, for example, DMED, which cleaves the backbone at the abasic site. For example, the combined action of the N-glycosylase (e.g., UNG or UDG) and DMED can result in the fragmentation of the first strand cDNA comprising the non canonical nucleotide (dU) to generate fragments comprising a blocked 3′-end, without affecting the second strand cDNA, which does not comprise non canonical nucleotides.

The single stranded second strand cDNA produced by the above reaction can comprise sequence B at the 5′-end, a sequence homologous to the RNA target, or a fraction thereof, and a sequence A′ at the 3′-end. Sequences B and A′ can be known and can be employed for further manipulation of the single stranded second strand cDNA. For example, the single stranded second strand cDNA can be rendered dsDNA by hybridization of an oligonucleotide comprising sequence A or part thereof, and extension of the hybridized oligonucleotide by DNA polymerase along the second strand cDNA template. Furthermore, the second strand cDNA can be amplified using primers that can hybridize to the end sequences, for example, by PCR. Solution phase PCR, emulsion PCR, surface bound PCR, such as Bridging-PCR and the like, are possible using the said single stranded DNA product or products. The primers can further comprise tail sequences which render the amplification product ends suitable for analysis on various analytical platforms, such as highly parallel sequencing (next generation sequencing, NGS). Any of the next generation sequencing techniques described herein can be used to sequence the second strand cDNA (e.g., Genome Analyzer by Illumina, the Genome Sequencer by 454/Roche Lifesciences, the SMRT platform by Pacific Biosciences, the HeliScope by Helicos Biosciences, the Polonator by Dover Systems, and semiconductor sequencing (Personal Genome Machine (PGM™) by Ion Torrent). The specific desired sequences can be appended at the end of the dsDNA product by ligation.

Direct application of the single stranded cDNA with desired 3′- and 5′-end sequences is also possible, when the end sequences are compatible with the chosen sequencing system (next generation sequencing (NGS) for example).

The end sequences flanking the sequence homologous to the RNA target can comprise molecular barcodes, when used for multiplex analysis, wherein specific barcodes can be designed for generation of the above described single stranded cDNA from individual samples followed by pooling of a number of products derived from individual samples.

The end sequence can comprise recognition sequences for restriction enzymes, thus enabling cleavage of the ends by the desired restriction enzyme, leading to the generation of ends suitable for further manipulations. For example, double stranded cDNA comprising desired overhangs at the two ends can be generated.

The defined known ends flanking the single stranded cDNA can further enable circularization by hybridizing an oligonucleotide complementary to the end sequences and ligation of the ends. The circular cDNA can be further amplified by rolling circle amplification (RCA).

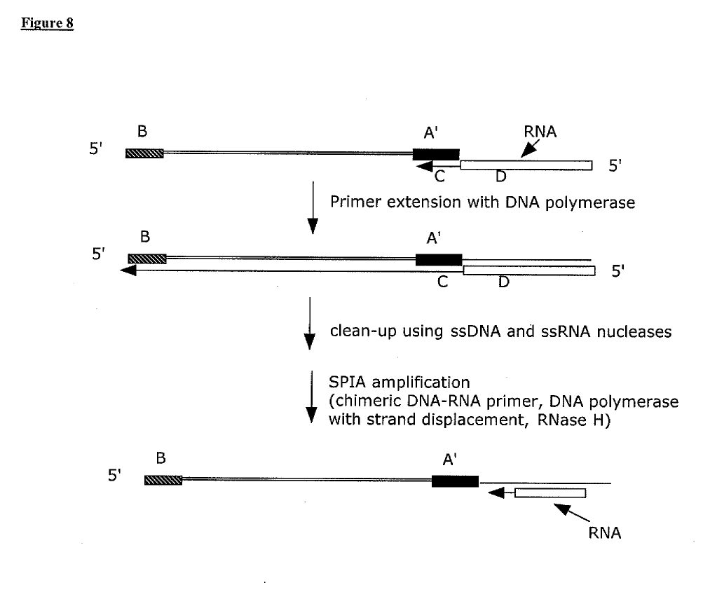

The second strand cDNA with the defined 5′- and 3′-ends can be also amplified by single primer isothermal amplification (SPIA) (FIG. 8) (see, e.g., U.S. Patent Application Publication No. 20090130721). For example, the single stranded second strand cDNA can be hybridized to a first composite primer, wherein the first composite primer comprises a 3′ portion (e.g., DNA) which can anneal to the single stranded second strand cDNA and a 5′ portion, which can be RNA, e.g., which does not hybridize to the single stranded second strand cDNA. The first composite primer can be extended with DNA polymerase. The second strand cDNA can be extended, using the RNA portion of the first composite primer as a template, with an RNA dependent DNA polymerase, to form a DNA/RNA heteroduplex. An agent that cleaves single-stranded RNA, e.g., RNase I, can be used to degrade the first composite primer that did not anneal to the second strand cDNA. This degradation can be used to prevent competition with other primers for subsequent annealing and to increase the efficiency of the subsequent single primer isothermal amplification reaction. The enzyme that is capable of cleaving single-stranded RNA may be, for example, RNase I, RNase T, RNase A, or combination thereof. Generally, an enzyme with little or no sequence specificity, and an enzyme that does not cleave the RNA in an RNA/DNA heteroduplex, can be used to cleave single-stranded RNA. In some embodiments, the enzyme capable of cleaving single-stranded RNA is RNase I. In one embodiment, the RNase I is inactivated by heat.

The RNA component of the RNA/DNA heteroduplex can be degraded by an enzyme that degrades RNA in an RNA/DNA heteroduplex, e.g., RNase H, to generate a 3′ overhang. The enzymes that degrade RNA can be inactivated. Next, a composite amplification primer can hybridize to the 3′ overhang of the second strand cDNA, wherein the composite amplification primer can comprises an RNA portion and a 3′ DNA portion. The composite amplification primer hybridized to the 3′ overhang of the second strand cDNA can be extended with at least one enzyme comprising DNA-dependent DNA polymerase activity. The first primer extension product can be displaced, RNA can be cleaved from the composite amplification primer and another composite amplification primer can hybridize such that primer extension and strand displacement are repeated, and whereby multiple copies of a polynucleotide sequence complementary to the RNA sequence of interest are generated.

An input RNA target (template) can be total RNA from a biological sample. The RNA can be purified from a sample lysate or can be purified from cell free fluids, such as, e.g., plasma or serum. The sample can be cell lysate comprising RNA and DNA.

The RNA target can be fragmented RNA. RNA fragmentation can result from purification from a fixed sample, such as a Formalin-Fixed, Paraffin-Embedded (FFPE) sample, or can be carried out intentionally, for example, by heating in the presence of multivalent cation. The RNA can come from a fresh, frozen sample. An input template nucleic acid can be derived, e.g., from whole blood, frozen sample, sorted cells, FFPE sample, a tissue biopsy, serum, or skin.

The products of a linear amplification, or the primer extension to produce double stranded cDNA with desired sequence at the two ends, can be produced in reaction mixtures comprising non canonical nucleotide triphosphate, for example dUTP, thus generating a strand which is cleavable (for example, by combined reaction of UNG and DMED). Thus, following further manipulation of the products the synthesized strand can be removed by cleavage.

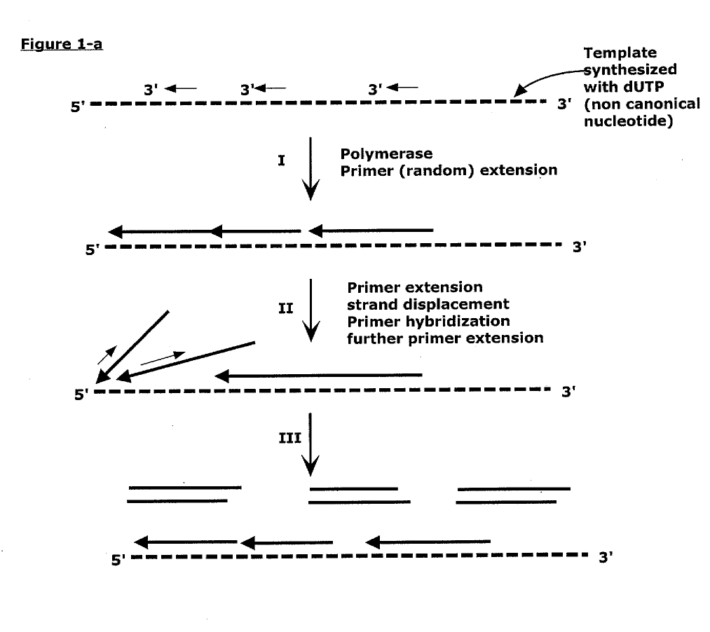

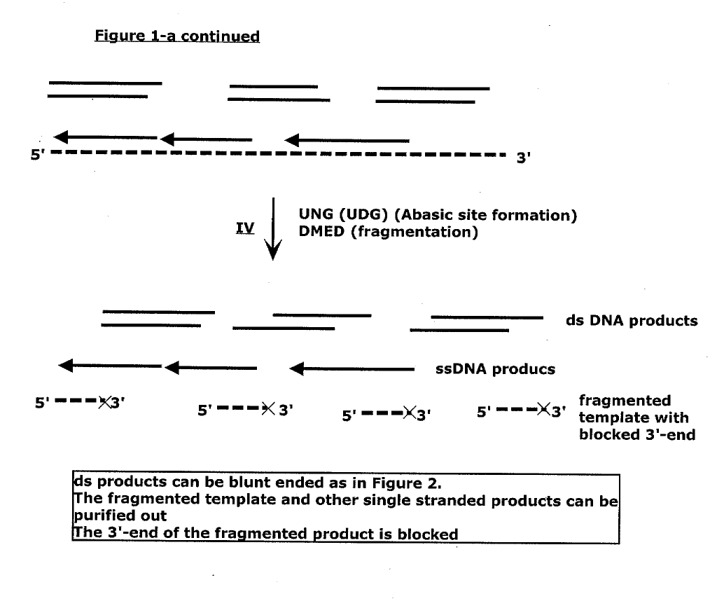

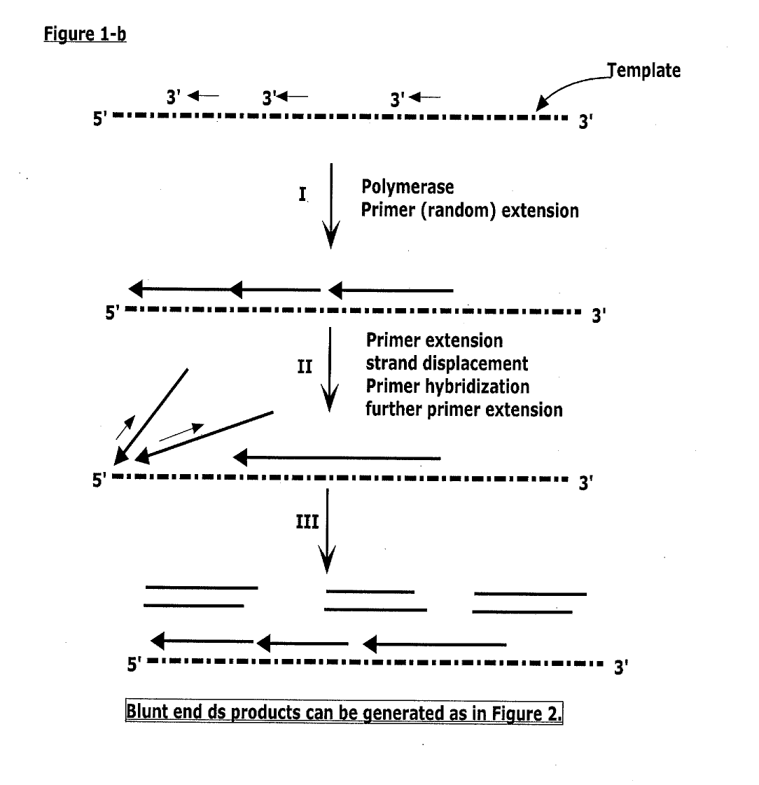

FIG. 1a depicts methods and compositions in one embodiment of the invention for generating double stranded nucleic acid products from one or more template polynucleotides utilizing a plurality of oligonucleotides comprising random hybridizing portions and a polymerase comprising strand displacement activity. In this embodiment the products are generated from a template that contains one or more non-canonical nucleotides (dUTP). The figure further depicts methods and compositions for fragmenting the template polynucleotide and removing undesired single stranded nucleic acids.

FIG. 1b depicts methods and compositions in another embodiment of the invention for generating double stranded nucleic acid products from one or more template polynucleotides utilizing a plurality of oligonucleotides comprising random hybridizing portions and a polymerase comprising strand displacement activity. In this embodiment the template nucleotide does not contain any non-canonical nucleotides. Methods and compositions for fragmenting the template polynucleotide and removing undesired single stranded nucleic acids can be used.

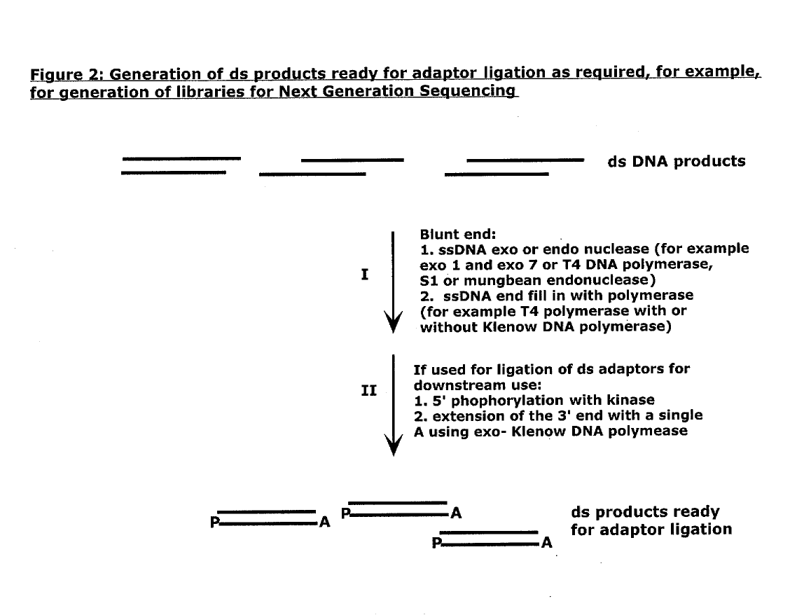

FIG. 2 depicts methods and compositions for preparing double stranded nucleic acid for adaptor ligation.

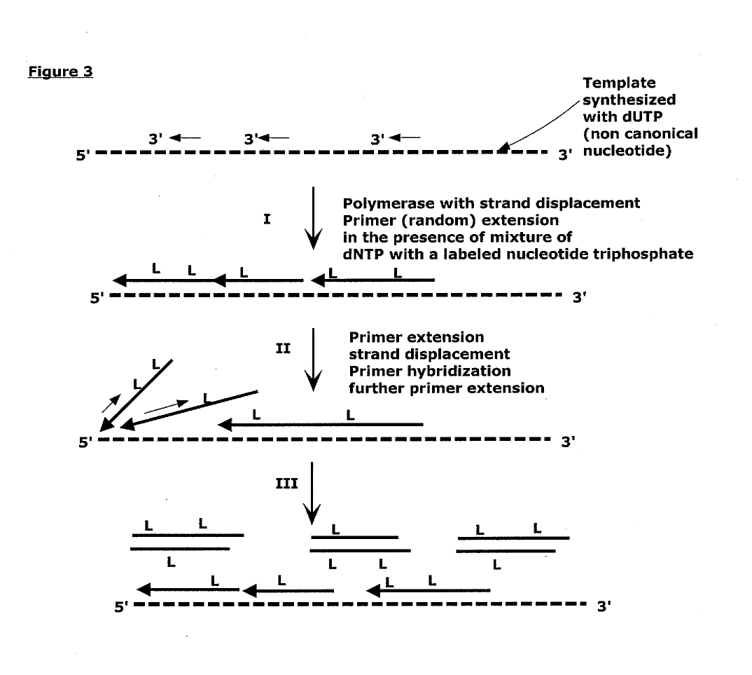

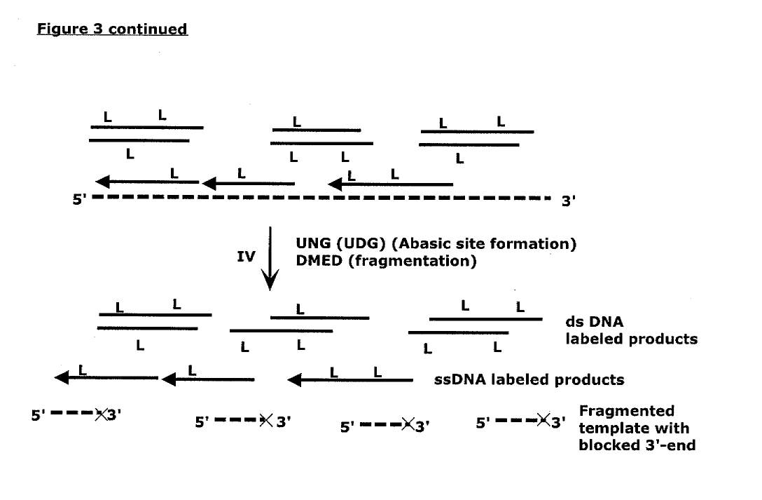

FIG. 3 depicts methods and compositions for generating labeled double stranded nucleic acid products from one or more template polynucleotides utilizing a plurality of oligonucleotides comprising one or more labels and random hybridizing portions and a polymerase comprising strand displacement activity. The figure further predicts methods and compositions for fragmenting the template polynucleotide. Undesired single stranded nucleic acids can be removed.

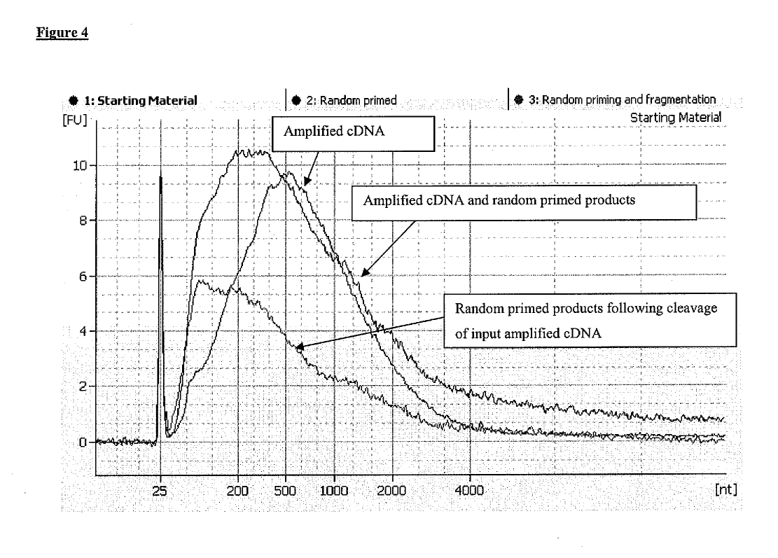

FIG. 4 depicts BioAnalyzer profiles of input amplified cDNA, random primed products mixture and random primed products following cleavage of the input amplified cDNA. Whole transcriptome amplified cDNA from HeLa total RNA. The x-axis represents product length in bp. The concentration of the starting material is 172 ng/μL. The concentration of random primed amplified DNA is 200 ng/μL. The concentration of the random primed and fragmented input nucleic acid is 100 ng/μL.

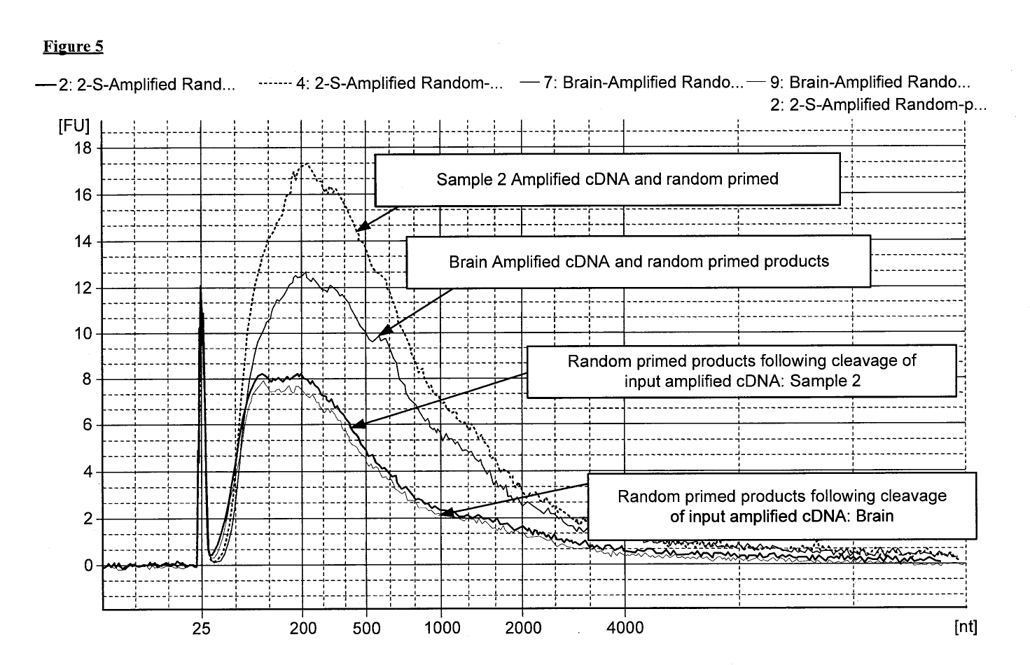

FIG. 5 depicts BioAnalyzer profiles of input amplified cDNA, random primed products mixture and random primed products following cleavage of the input amplified cDNA. Whole transcriptome amplified cDNA from total RNA from a biological sample and brain is used. The x-axis represents product length in bp. The profile of the random priming products following removal of the input amplified cDNA is very reproducible for the two samples which represent different sample source.

### III. Compositions of the Invention

Appropriate reaction media and conditions for carrying out the methods of the invention are those that permit nucleic acid preparation according to the methods of the invention. Such media and conditions are known to persons of skill in the art, and are described in various publications, such as U.S. Pat. Nos. 5,554,516; 5,716,785; 5,130,238; 5,194,370; 6,090,591; 5,409,818; 5,554,517; 5,169,766; 5,480,784; 5,399,491; 5,679,512; and PCT Pub. No. WO 99/42618. For example, a buffer may be Tris buffer, although other buffers can also be used as long as the buffer components are non-inhibitory to enzyme components of the methods of the invention. The pH is preferably from about 5 to about 11, more preferably from about 6 to about 10, even more preferably from about 7 to about 9, and most preferably from about 7.5 to about 8.5. The reaction medium can also include bivalent metal ions such as Mg2+ or Mn2+, at a final concentration of free ions that is within the range of from about 0.01 to about 15 mM, and most preferably from about 1 to 10 mM. The reaction medium can also include other salts, such as KCl or NaCl, that contribute to the total ionic strength of the medium. For example, the range of a salt such as KCl is preferably from about 0 to about 125 mM, more preferably from about 0 to about 100 mM, and most preferably from about 0 to about 75 mM. The reaction medium can further include additives that could affect performance of the amplification or primer extension reactions, but that are not integral to the activity of the enzyme components of the methods. Such additives include proteins such as BSA, single strand binding proteins (e.g., T4 gene 32 protein), and non-ionic detergents such as NP40 or Triton. Reagents, such as DTT, that are capable of maintaining enzyme activities can also be included. Such reagents are known in the art. Where appropriate, an RNase inhibitor (such as RNasin) that does not inhibit the activity of an RNase employed in the method can also be included. Any aspect of the methods of the invention can occur at the same or varying temperatures. Amplification or primer extension reactions may be performed isothermally, which avoids the cumbersome thermocycling process, or may be performed by standard thermocycling techniques such as PCR. The amplification or primer extension reaction may be carried out at a temperature that permits hybridization of the oligonucleotides primers of the invention to the template polynucleotide and primer extension products, and that does not substantially inhibit the activity of the enzymes employed. The temperature can be in the range of preferably about 25° C. to about 85° C., more preferably about 30° C. to about 80° C., and most preferably about 37° C. to about 75° C. In some embodiments that include RNA transcription, the temperature for the transcription steps is lower than the temperature(s) for the preceding steps. In these embodiments, the temperature of the transcription steps can be in the range of preferably about 25° C. to about 85° C., more preferably about 30° C. to about 75° C., and most preferably about 37° C. to about 70° C.

Nucleotide and/or nucleotide analogs, such as deoxyribonucleoside triphosphates, that can be employed for synthesis of the primer extension products or for amplification in the methods of the invention are provided in the amount of from preferably about 50 to about 2500 μM, more preferably about 100 to about 2000 μM, even more preferably about 200 to about 1700 μM, and most preferably about 250 to about 1500 μM. In some embodiments, a nucleotide or nucleotide analog whose presence in the primer extension strand enhances displacement of the strand (for example, by causing base pairing that is weaker than conventional AT, CG base pairing) is included. Such nucleotide or nucleotide analogs include deoxyinosine and other modified bases, all of which are known in the art. Nucleotides and/or analogs, such as ribonucleoside triphosphates, that can be employed for synthesis of the RNA transcripts in the methods of the invention are provided in the amount of from preferably about 0.25 to about 6 mM, more preferably about 0.5 to about 5 mM, even more preferably about 0.75 to about 4 mM, and most preferably about 1 to about 3 mM. Nucleotide analogues may further include non-canonical nucleotides such as for example methylated purine nucleotides, or dUTP. Additionally, nucleotide analogues may include labeled nucleotides such as amino-allyl labeled nucleotides, digoxin labeled nucleotides, fluorophore (e.g. Cy3, Cy5, fluoroscein, rhodamine) labeled nucleotides, biotin labeled nucleotides, or protein labeled nucleotides (e.g. avidin, streptavidin, neutravidin, horseradish peroxidase, alkaline phosphatase).

Modified nucleotides can be canonical nucleotide or non-canonical (cleavable) nucleotides. It is understood, however, that modified nucleotides that are not non-canonical (cleavable) nucleotide under the reaction conditions used in the methods of the invention, if present, generally should not affect the ability of the polynucleotide to undergo cleavage of a base portion of non-canonical nucleotide, such that an abasic site is generated, and/or cleavage of a phosphodiester backbone at an abasic site, such that fragments are generated, as described herein. If present, modification to the nucleotide structure, such as methylated nucleotides may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). It is understood that internucleotide modifications may, e.g., alter the efficiency and/or kinetics of cleavage of the phosphodiester backbone (as when, for example a phosphodiester backbone is cleaved at an abasic site, as described herein). Further, any of the hydroxyl groups ordinarily present in the sugars may be replaced, for example, by phosphonate groups, phosphate groups, protected by standard protecting groups, or activated to prepare additional linkages to additional nucleotides. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including DNA. It is understood, however, that modified nucleotides and/or internucleotide linkages and/or, if present, generally should not affect the ability of the polynucleotide to undergo cleavage of a base portion of a non-canonical nucleotide, such that an abasic site is generated, and/or the ability of a polynucleotide to undergo cleavage of a phosphodiester backbone at an abasic site, such that fragments are generated, as described herein.

In order to prepare primer extension products including single and double stranded DNA products, oligonucleotide primers may be used that bind to the input nucleic acid template and are subsequently extended along the template by one or more enzymes such as for example ligases or polymerases including but not limited to polymerases comprising strand displacement activity. The oligonucleotide components of the amplification reactions of the invention are generally in excess of the number of target nucleic acid sequence to be amplified. They can be provided at about or at least about any of the following: 1, 5, 10, 10², 10⁴, 10⁶, 10⁸, 10¹⁰, 10¹² times the amount of target nucleic acid. Oligonucleotide primers, composite primers and pro-promoter template oligonucleotides (PTO) can each be provided at about or at least about any of the following concentrations: 50 nM, 100 nM, 500 nM, 1000 nM, 2500 nM, 5000 nM. The primer may be complementary to at least a portion of the input nucleic acid template and may comprise DNA, RNA, labeled RNA or DNA, or mixtures thereof. The primers may comprise specific sequences, multiple sequences, or random sequences. Various embodiments of the primers used in the methods of the invention are described herein. For the methods described herein, one or more primers can be used.

In one embodiment, the oligonucleotide primers are oligonucleotides that are supplied externally. The primer in the methods of the invention comprises a sequence (which may or may not be the whole of the primer) that is hybridizable (under a given set of conditions) to the template nucleic acid such that any extension products would be homologous to the template nucleic acid. In another embodiment, the primer is a primer comprising DNA (in some embodiments, consisting of DNA).

To achieve hybridization to a template nucleic acid (which, as is well known and understood in the art, depends on other factors such as, for example, ionic strength and temperature), the sequence of the primer that is hybridizable to the template nucleic acid is preferably of at least about 60%, more preferably at least about 75%, even more preferably at least about 90%, and most preferably at least about 95% or 100% complementarity to the template nucleic acid.

Suitable primers in the methods of the invention are long enough such that they do not dissociate or do not dissociate substantially from the template nucleic acid in an uninduced manner (e.g. they do not dissociate in the absence of heat or chemical denaturation or strand displacement activity). Suitable primers are preferably from about 3 to about 200, more preferably from about 5 or 6 to about 150, even more preferably from about 10 to about 100, and most preferably from about 12 to about 50, nucleotides in length, or about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 nucleotides in length or longer.

In some embodiments, the one or more primers comprise random sequences. In other embodiments, the primer comprises a portion (for example, a 3′ portion) that comprises a random sequence (i.e., a sequence designed to be hybridizable (under a given set of conditions) to one or more sequences in the sample). In some cases the one or more primers comprise a polyT or a polyA sequence. In some cases, the primers comprise a mixture of random sequences and specific sequences such as polyT sequences or polyA sequences. In some cases, the primers are generated as a pool of random sequences and are further selected by hybridization or subtractive hybridization to remove sequences which hybridize to undesired sequences. In some embodiments, the primers comprise a pool of primers synthesized to comprise all desired sequences (e.g. designed to hybridize to all possible or substantially all possible desired sequences and not to hybridize to undesired sequences in the samples such as for example rRNA sequences.) This pool of sequences comprises a pool of semi-random or not so random sequences.

In some cases, non-standard bases which exhibit base pairing interactions with more than one partner such as inosine which can base pair with uracil, adenine, and cytosine may be used. The non-standard bases with greater propensity for multiple base pairing partners are referred to as wobble bases. In other cases, specific annealing sequences may be used in combination with such wobble bases. In some cases, primers comprise specific annealing sequences in which one or more positions within the annealing sequence are randomized. Such a combination of specific sequences with randomized positions may be used for example to amplify a set of related or homologous nucleic acid sequences. In still other cases, primers comprise specific annealing sequences, or a set of specific sequences.

In some embodiments, the primer comprises a 5′ portion that is non-hybridizable to a template nucleic acid (tailed primer). In some cases, the 5′-region of the primer further comprises defined or universal sequences. In some cases, a primer of the present invention may comprise a linker region that is not a hybridizable portion and is not a tail. Primers may also comprise a ligand. The ligand may or may not be comprised of nucleic acid or a peptide. In some cases, the ligand is a small organic molecule such as for example biotin or a fluorophore. The primers may be synthesized by a number of common methods known in the art. In some cases, the methods of synthesizing primers of the present invention include solid phase synthesis methods such as provided in U.S. Pat. No. 5,623,068.

In some aspects of the present invention, composite primers are utilized, such as for example during generation of a template nucleic acid comprising one or more non-canonical nucleotide or for amplification or labeling of the products of the primer extension step of the present invention. Composite primers of the present invention include primers that comprise an RNA segment and a DNA segment. The RNA segment may be generally located at the 5′ end of the composite primer and the DNA segment may generally be located at the 3′ end of the composite c primer. In some cases the RNA and DNA segments are adjacent. In other cases, there is an intervening element between the RNA and the DNA segment. The intervening element may comprise additional nucleic acid sequence. In one embodiment, a portion of the DNA segment comprises a template annealing sequence. The template annealing sequence as described primers provided herein may be target specific, a set of target specific sequences, non-random, not-so random, or a random sequence.

In some embodiments of the present invention, the DNA-RNA composite primer annealing sequences may be between about 2 nucleotides in length to about 100 nucleotides in length. In some cases, annealing sequences may be between about 3 nucleotides in length to about 50 nucleotides in length. In still other cases, annealing sequences may be about 4 to about 30 nucleotides in length. For example, but without limitation, DNA-RNA composite primer annealing sequences may be about 5, 6, 7, 8, 9, 10, 12, 14, 16, 18, 20, or 25 nucleotides in length. In some cases, random annealing sequences comprise random pentamers, hexamers, heptamers, octomers, decamers, undecamers, dodecamers, tirdecamers, or larger.

In some embodiments of the present invention, a portion of the RNA segment of the composite primer comprises a sequence tail. The sequence tail may in some cases comprise the same sequence as the sequence tail used for a previous primer extension reaction or a previous amplification reaction. In other cases, the sequence tail may comprise a different sequence as compared to the sequence tail used for a previous primer extension reaction or amplification reaction. In still other cases, the sequence tail may be complementary to a sequence tail used in a previous primer extension reaction or a previous amplification reaction.

In some embodiments of the present invention, a composite primer comprising a 5′-RNA segment comprising a sequence tail and a 3′ DNA segment comprising an annealing sequence (e.g. a random annealing sequence) is annealed to a primer extension product (e.g. a single stranded primer extension product, or a partial double stranded product comprising a portion that is double stranded and a portion that is single stranded), a polymerase is added to the annealing reaction, and a double stranded product is created comprising the primer extension product and a composite primer extension product. In some cases, the polymerase may comprise stand displacement activity. The double stranded product may be denatured and another primer may be annealed to the composite primer extension product and extended with a polymerase to create a double stranded product comprising an RNA/DNA heteroduplex. In some embodiments of the present invention, the double stranded product comprising an RNA/DNA heteroduplex is used as a substrate for amplification via SPIA using a chimeric amplification primer as provided herein. Alternatively, the double stranded product may be amplified using any other amplification method provided herein.

In some cases, the RNA portion of the RNA/DNA heteroduplex may be cleaved by an agent that cleaves the RNA portion of an RNA/DNA heteroduplex. The agent may be for example an RNase H or a polymerase or other enzyme that comprises RNase H-like cleavage activity. In some cases an amplification primer such as a chimeric amplification primer (e.g. an RNA DNA composite primer suitable for use as an amplification primer) may be annealed to the 3′ ssDNA portion provided by the step of cleaving the RNA portion of the RNA/DNA heteroduplex. In some cases, a DNA polymerase, such as a DNA polymerase with strand displacement activity may be used to extend the hybridized chimeric amplification primer in a reaction mixture to generate a double stranded product comprising an RNA/DNA heteroduplex and a single stranded product of SPIA amplification. In some cases, the reaction mixture may comprise a chimeric amplification primer, an agent that cleaves the RNA portion of an RNA/DNA heteroduplex and a DNA polymerase comprising strand displacement activity and various buffers, salts, and reagents (e.g. dNTPs) for amplification of the double stranded SPIA substrate comprising an RNA/DNA heteroduplex as provided herein. In some cases, the amplification of the SPIA substrate may be performed isothermally.

Amplification primers of the present invention include chimeric amplification primers. Chimeric amplification primers are RNA/DNA composite primers that can be used to create multiple copies of (amplify) a polynucleotide sequence isothermally using RNA cleavage, and DNA polymerase activity with strand displacement. Amplification with such primers is described, for example in U.S. Pat. Nos. 6,251,639, 6,692,918, and 6,946,251. The composite amplification primer comprises sequences capable of hybridizing to a portion of a DNA template, and most often comprises sequences hybridizable to a defined 3′-portion of the DNA. Amplification primers of the present invention may also comprise all DNA canonical or non canonical nucleotides.

In some embodiments, a chimeric amplification primer comprises at least one RNA portion that is capable of binding (hybridizing) to a sequence on a DNA template and at least one DNA portion that is capable of binding (hybridizing) to a sequence on a DNA template, wherein the RNA portion is susceptible to being cleaved with an RNase H when hybridized to the DNA template. The chimeric amplification primers bind to the DNA template to form a partial heteroduplex in which only the RNA portion of the primer is cleaved upon contact with a ribonuclease such as RNase H, while the DNA template remains intact, thus enabling annealing of another chimeric primer. In some aspects of the invention, the 5′ RNA portion of the chimeric amplification primer may be about 4, 5, 6, 7, 8, 9, 10, 11, 12, 13, 14, 15, 16, 17, 18, 19, 20, 21, 22, 23, 24, 25, 26, 27, 28, 29, 30, or about 40 nucleotides in length.

The chimeric amplification primers may also comprise a 3′-DNA portion that is capable of hybridization to a sequence on the DNA template such that its hybridization to the DNA is favored over that of the nucleic acid strand that is displaced from the DNA template by the DNA polymerase. Such primers can be rationally designed based on well known factors that influence nucleic acid binding affinity, such as sequence length and/or identity, as well as hybridization conditions. In some aspects of the present invention, the 3′ DNA portion of a composite amplification primer may be about 4, 5, 6, 7, 8, 9, 10, 11, 12, 13, 14, 15, 16, 17, 18, 19, or 20 nucleotides in length.

In some embodiments of the present invention chimeric oligonucleotides are utilized. In some cases, the chimeric oligonucleotides may be used to provide a defined end sequence for the SPIA amplified DNA products. The chimeric oligonucleotides may comprise a 5′ segment comprising RNA and a 3′ segment comprising DNA. In some embodiments, a portion of the 3′ segment, of the chimeric oligonucleotide is substantially the same as the RNA sequence removed from the DNA-RNA heteroduplex of a SPIA substrate by RNase H.

Primers of the present invention may be useful for the primer extension step of the present invention for producing primer extension products that do not comprise non-canonical nucleotides (e.g. single or double stranded nucleic acids or a combination thereof) or that do not comprise the same non-canonical nucleotides as a template nucleic acid, and that may be differentially separated from template nucleic acid comprising one or more non-canonical nucleotides via a step of cleaving the template nucleic acid. Primers of the present invention may also be useful for the generation of template nucleic acid comprising one or more non-canonical nucleotides, such as for example by an amplification or polymerization step including any of the amplification or polymerization methods provided herein including PCR, SPIA, IVT, reverse transcription or a combination thereof, or by ligation of a plurality of adjacent primers, or a combination thereof. In some cases, primers are useful for the generation of template nucleic acid comprising non-canonical nucleotides via a limited amplification method using a single round of amplification or a small number of rounds (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, etc.) such as in the generation of cDNA. Primers of the present invention may also be useful for amplifying or labeling the products of the primer extension reaction. For example, primers of the present invention may be used for further amplification of the products of the primer extension reaction after the cleavage of the template nucleic acid, purification or a combination thereof, such as by PCR, SPIA, IVT, any amplification method provided herein, or a combination thereof.

Cleavage agents of the present invention include enzymes such as for example glycosylases (e.g. UDG or MPG) and endonucleases (e.g. AP endonucleases), and chemical agents such as for example bases, amines, primary amines, secondary amines, tertiary amines, polyamines (e.g. spermine, spermidine, 1,4-diaminobutane, lysine, the tripeptide K—W—K, N,N-dimethylethylenediamine (DMED), piperazine, or 1,2-ethylenediamine) and nucleophiles or any combination thereof. Cleavage agents comprising enzymes may be used in a reaction mixture at various concentrations suitable for the methods of the present invention including between about 1 nM and about 1 mM, or between about 5 nM and about 0.5 mM, about 10 nM and about 0.25 mM, about 25 nM and about 0.1 mM, about 50 nM and about 0.05 mM, about 100 nM and about 0.025 mM, about 150 nM and about 0.0125 mM, about 200 nM and about 0.01 mM, about 300 nM and about 5 μM, about 500 nM and about 2 μM, or between about 0.75 μM and about 1 μM. In some cases, cleavage agents comprising enzymes may be used in a reaction mixture at about 1 nM, 2 nM, 3 nM, 4 nM, 5 nM, 7.5 nM, 10 nM, 15 nM, 20 nM, 25 nM, 30 nM, 35 nM, 50 nM, 75 nM, 100 nM, 150 nM, 200 nM, 250 nM, 500 nM, 750 nM, 1 μM, 2 μM, 3 μM, 5 μM, 7.5 μM, 10 μM, 25 μM, 50 μM, 100 μM or more. Cleavage agents comprising chemical agents may be used in a reaction mixture at various suitable for the methods of the present invention including between about 1 nM and about 1 mM, or between about 5 nM and about 0.5 mM, about 10 nM and about 0.25 mM, about 25 nM and about 0.1 mM, about 50 nM and about 0.05 mM, about 100 nM and about 0.025 mM, about 150 nM and about 0.0125 mM, about 200 nM and about 0.01 mM, about 300 nM and about 5 μM, about 500 nM and about 2 μM, or between about 0.75 μM and about 1 μM. In some cases, cleavage agents comprising chemical agents may be used in a reaction mixture at about 1 nM, 2 nM, 3 nM, 4 nM, 5 nM, 7.5 nM, 10 nM, 15 nM, 20 nM, 25 nM, 30 nM, 35 nM, 50 nM, 75 nM, 100 nM, 150 nM, 200 nM, 250 nM, 500 nM, 750 nM, 1 μM, 2 μM, 3 μM, 5 μM, 7.5 μM, 10 μM, 25 μM, 50 μM, 100 μM or more.

The initial step of the methods of the present invention may be providing the template nucleic acid, which template comprises one or more non-canonical nucleotides. The non-canonical nucleotides may be incorporated by synthetic (e.g. chemical synthesis) or enzymatic means (e.g. incorporation by ligase or polymerase), or a combination thereof.

The input nucleic acid template is generally a polymeric nucleotide, which in the intact natural state can have about 30 to 5,000,000 to about 3×10⁹ or more nucleotides and in an isolated state can have about 20 to 100,000 or more nucleotides, usually about 100 to 50,000 nucleotides, more frequently 500 to 20,000 nucleotides, or about 500 to 10,000 nucleotides. The template includes RNAs or DNAs from any source and or/species, including human, animals, plants, and microorganisms such as bacteria, yeasts, viruses, viroids, molds, fungi, plants, and fragments thereof. Exemplary templates can be obtained and purified using standard techniques in the art and includes RNAs and DNAs in purified or unpurified form, which include, but are not limited to, mRNAs, tRNAs, snRNAs, rRNAs, retroviruses, small non-coding RNAs, microRNAs, polysomal RNAs, pre-mRNAs, intronic RNA, viral RNA, DNA-RNA heteroduplexes, DNA, genomic DNA, and fragments thereof. Use of a DNA template in the methods of the present invention (including genomic DNA target) may require initial reverse transcription of an RNA target into DNA form, which can be achieved using methods known in the art. Preparation of a RNA/DNA heteroduplex, double stranded RNA, sequences prone to stem loop formation, or double stranded DNA may require denaturation of the duplex to obtain a single stranded nucleic acid, or denaturation followed by reverse transcription of the RNA strand to obtain an DNA. The template may be only a minor fraction of a complex mixture such as a biological sample and may be obtained from various biological materials by procedures known in the art.

The template nucleic acid can be known or unknown and may contain more than one desired or suspected specific nucleic acid sequence of interest, each of which may be the same or different from each other. Therefore, the methods provided herein are useful not only for preparing double stranded DNA fragments from one specific nucleic acid sequence, but also for preparing double stranded DNA fragments simultaneously more than one different specific nucleic acid sequence located on the same or different nucleic acid molecules.

Enzymes of the present invention include but are not limited to one or more polymerases, one or more endonucleases, one or more exonucleases, one or more ligases, one or more nucleotide kinases, one or more glycosidases, one or more RNases, including but not limited to RNaseI or RNase H, or any combination thereof. Polymerases of the present invention include RNA dependent and DNA dependent polymerases. Polymerases further include RNA and DNA dependent DNA polymerases as well as RNA and DNA dependent RNA polymerases. Accordingly, polymerases include reverse transcriptases, and transcriptases. In some embodiments, polymerases of the present invention comprise both RNA dependent and DNA dependent DNA polymerase activity. In some cases, polymerases of the present invention include proof reading activity. Some of the polymerases of the present invention may be useful for degradation of 3′ or 5′ ends or both. Some of the polymerases of the present invention may be useful for filling in of single stranded overhangs of double stranded nucleic acids. Some of the polymerases of the present invention may be useful for forming blunt ends from double stranded nucleic acids comprising single stranded overhangs from one or more of their 3′ and 5′ ends either by trimming back the overhangs (degrading the single stranded portion), or by filling in the overhang (polymerizing a sequence complementary and hybridized to the single stranded overhang).

Polymerases may be used in a reaction mixture at various concentrations suitable for the methods of the present invention including between about 1 nM and about 1 mM, or between about 5 nM and about 0.5 mM, about 10 nM and about 0.25 mM, about 25 nM and about 0.1 mM, about 50 nM and about 0.05 mM, about 100 nM and about 0.025 mM, about 150 nM and about 0.0125 mM, about 200 nM and about 0.01 mM, about 300 nM and about 5 μM, about 500 nM and about 2 μM, or between about 0.75 μM and about 1 μM. In some cases, polymerases may be used in a reaction mixture at about 1 nM or more, 2 nM or more, 3 nM or more, 4 nM or more, 5 nM or more, 7.5 nM or more, 10 nM or more, 15 nM or more, 20 nM or more, 25 nM or more, 30 nM or more, 35 nM or more, 50 nM or more, 75 nM or more, 100 nM or more, 150 nM or more, 200 nM or more, 250 nM or more, 500 nM or more, 750 nM or more, 1 μM or more, 2 μM or more, 3 μM or more, 5 μM or more, 7.5 μM or more, 10 μM or more, 25 μM or more, 50 μM or more, 100 μM or more.

Exemplary polymerase include mesophilic and thermophilic polymerases including but not limited to Klenow polymerase, exo-Klenow polymerase, 3′ 5′ exo-Klenow polymerase, 3′ exo-Klenow polymerase, 5′ exo-Klenow polymerase, DNA polymerase 1, Bst polymerase, Bst large fragment polymerase, Bca Polymerase and derivatives thereof, Vent polymerase, Vent polymerase, Deep Vent (exo-) polymerase, 9° Nm polymerase, Therminator polymerase, Therminator II polymerase, MMulV Reverse Transcriptase, phi29 polymerase, T4 DNA polymerase, T4 RNA polymerase, or DyNAzyme EXT polymerase, Phusion Polymerase, or any other polymerases known in the art or any combination thereof. An “RNA-dependent DNA polymerase” or “reverse transcriptase” (“RT”) is an enzyme that synthesizes a complementary DNA copy from an RNA template. All known reverse transcriptases also have the ability to make a complementary DNA copy from a DNA template; thus, they are both RNA- and DNA-dependent DNA polymerases. Reverse transcriptases may also have an RNase H activity. Some examples of reverse transcriptases are reverse transcriptase derived from Maloney murine leukemia virus (MMLV-RT), avian myeloblastosis virus, retroviral reverse transcriptase, retrotransposon reverse transcriptase, hepatitis B reverse transcriptase, cauliflower mosaic virus reverse transcriptase, bacterial reverse transcriptase, E. coli DNA polymerase and klenow fragment, and Tth DNA polymerase. A primer can be used to initiate synthesis with both RNA and DNA templates. In other examples a DNA dependent DNA polymerase may also comprise an RNA-dependent DNA polymerase such as Klenow polymerase, Bst DNA polymerase and the like.

Ligases of the present invention include thermophilic and mesophilic ligases including but not limited to T4 DNA ligase, T4 RNA ligase, Taq ligase, E. coli ligase, Tfi ligase, and Ampligase®, or any other ligases known in the art or combinations thereof Ligases may be used in a reaction mixture at various concentrations suitable for the methods of the present invention including between about 1 nM and about 1 mM, or between about 5 nM and about 0.5 mM, about 10 nM and about 0.25 mM, about 25 nM and about 0.1 mM, about 50 nM and about 0.05 mM, about 100 nM and about 0.025 mM, about 150 nM and about 0.0125 mM, about 200 nM and about 0.01 mM, about 300 nM and about 5 μM, about 500 nM and about 2 μM, or between about 0.75 μM and about 1 μM. In some cases, ligases may be used in a reaction mixture at about 1 nM or more, 2 nM or more, 3 nM or more, 4 nM or more, 5 nM or more, 7.5 nM or more, 10 nM or more, 15 nM or more, 20 nM or more, 25 nM or more, 30 nM or more, 35 nM or more, 50 nM or more, 75 nM or more, 100 nM or more, 150 nM or more, 200 nM or more, 250 nM or more, 500 nM or more, 750 nM or more, 1 μM or more, 2 μM or more, 3 μM or more, 5 μM or more, 7.5 μM or more, 10 μM or more, 25 μM or more, 50 μM or more, 100 μM or more.

Kinases of the present invention include thermophilic and mesophilic nucleotide kinases including but not limited to T4 polynucleotide kinase or any other nucleotide kinases known in the art or any combination thereof. Nucleotide kinases may be used in a reaction mixture at various concentrations suitable for the methods of the present invention including between about 1 nM and about 1 mM, or between about 5 nM and about 0.5 mM, about 10 nM and about 0.25 mM, about 25 nM and about 0.1 mM, about 50 nM and about 0.05 mM, about 100 nM and about 0.025 mM, about 150 nM and about 0.0125 mM, about 200 nM and about 0.01 mM, about 300 nM and about 5 μM, about 500 nM and about 2 μM, or between about 0.75 μM and about 1 μM. In some cases, nucleotide kinases may be used in a reaction mixture at about 1 nM or more, 2 nM or more, 3 nM or more, 4 nM or more, 5 nM or more, 7.5 nM or more, 10 nM or more, 15 nM or more, 20 nM or more, 25 nM or more, 30 nM or more, 35 nM or more, 50 nM or more, 75 nM or more, 100 nM or more, 150 nM or more, 200 nM or more, 250 nM or more, 500 nM or more, 750 nM or more, 1 μM or more, 2 μM or more, 3 μM or more, 5 μM or more, 7.5 μM or more, 10 μM or more, 25 μM or more, 50 μM or more, 100 μM or more.

Exonucleases of the present invention include thermophilic and mesophilic exonucleases including but not limited to single strand specific exonucleases, single stranded DNA specific exonucleases, RNA exonucleases, exonuclease 1, exonuclease 7, any other exonucleases known in the art, or any combination thereof. Exonucleases may be used in a reaction mixture at various concentrations suitable for the methods of the present invention including between about 1 nM and about 1 mM, or between about 5 nM and about 0.5 mM, about 10 nM and about 0.25 mM, about 25 nM and about 0.1 mM, about 50 nM and about 0.05 mM, about 100 nM and about 0.025 mM, about 150 nM and about 0.0125 mM, about 200 nM and about 0.01 mM, about 300 nM and about 5 μM, about 500 nM and about 2 μM, or between about 0.75 μM and about 1 μM. In some cases, exonucleases may be used in a reaction mixture at about 1 nM or more, 2 nM or more, 3 nM or more, 4 nM or more, 5 nM or more, 7.5 nM or more, 10 nM or more, 15 nM or more, 20 nM or more, 25 nM or more, 30 nM or more, 35 nM or more, 50 nM or more, 75 nM or more, 100 nM or more, 150 nM or more, 200 nM or more, 250 nM or more, 500 nM or more, 750 nM or more, 1 μM or more, 2 μM or more, 3 μM or more, 5 μM or more, 7.5 μM or more, 10 μM or more, 25 μM or more, 50 μM or more, 100 μM or more.

Endonucleases of the present invention include thermophilic and mesophilic endonucleases including but not limited to single strand specific endonucleases, single stranded DNA specific endonucleases, RNA endonucleases, S1 endonuclease, mung bean endonucleases, and any other endonucleases known in the art, or any combination thereof. Endonucleases may be used in a reaction mixture at various concentrations suitable for the methods of the present invention including between about 1 nM and about 1 mM, or between about 5 nM and about 0.5 mM, about 10 nM and about 0.25 mM, about 25 nM and about 0.1 mM, about 50 nM and about 0.05 mM, about 100 nM and about 0.025 mM, about 150 nM and about 0.0125 mM, about 200 nM and about 0.01 mM, about 300 nM and about 5 μM, about 500 nM and about 2 μM, or between about 0.75 μM and about 1 μM. In some cases, exonucleases may be used in a reaction mixture at about 1 nM or more, 2 nM or more, 3 nM or more, 4 nM or more, 5 nM or more, 7.5 nM or more, 10 nM or more, 15 nM or more, 20 nM or more, 25 nM or more, 30 nM or more, 35 nM or more, 50 nM or more, 75 nM or more, 100 nM or more, 150 nM or more, 200 nM or more, 250 nM or more, 500 nM or more, 750 nM or more, 1 μM or more, 2 μM or more, 3 μM or more, 5 μM or more, 7.5 μM or more, 10 μM or more, 25 μM or more, 50 μM or more, 100 μM or more.

Glycosylases of the present invention include but are not limited to glycosylases capable of cleaving a nucleotide to generate an abasic site in a polynucleotide substrate. In some embodiments, glycosylases of the present invention provide for specific cleavage of incorporated non-canonical nucleotides. Exemplary glycosylases include but are not limited to UNG/UDG, MPG, any other glycosylases such as those described in international application publication number WO/2008/005459, or U.S. application Ser. Nos. 10/441,663, and 11/026,280, or any combination thereof. Glycosidases may be used in a reaction mixture at various concentrations suitable for the methods of the present invention including between about 1 nM and about 1 mM, or between about 5 nM and about 0.5 mM, about 10 nM and about 0.25 mM, about 25 nM and about 0.1 mM, about 50 nM and about 0.05 mM, about 100 nM and about 0.025 mM, about 150 nM and about 0.0125 mM, about 200 nM and about 0.01 mM, about 300 nM and about SW, about 500 nM and about 2 μM, or between about 0.75 μM and about 1 μM. In some cases, glycosidases may be used in a reaction mixture at about 1 nM or more, 2 nM or more, 3 nM or more, 4 nM or more, 5 nM or more, 7.5 nM or more, 10 nM or more, 15 nM or more, 20 nM or more, 25 nM or more, 30 nM or more, 35 nM or more, 50 nM or more, 75 nM or more, 100 nM or more, 150 nM or more, 200 nM or more, 250 nM or more, 500 nM or more, 750 nM or more, 1 μM or more, 2 μM or more, 3 μM or more, 5 μM or more, 7.5 μM or more, 10 μM or more, 25 μM or more, 50 μM or more, 100 μM or more. In some cases, one glycosidase such as UDG or any glycosidase provided herein may be used to cleave the base portion of one or more non-canonical nucleotides in the template nucleic acid. In other cases more than one glycosidase such as any combination of the glycosidases provided herein may be used to cleave the base portion of one or more non-canonical nucleotides in the template nucleic acid. In some cases, the glycosidase used to cleave the base portion of one or more non-canonical nucleotides in the template nucleic acid to generate one or more abasic sites may also comprise a lyase activity for cleaving the phosphodiester backbone at the one or more abasic sites or a portion thereof.

### IV. Kits of the Invention

The present invention provides kits containing one or more compositions of the present invention and other suitable reagents suitable for carrying out the methods of the present invention. The invention provides, e.g., diagnostic kits for clinical or criminal laboratories, or nucleic acid amplification or analysis kits for general laboratory use. The present invention thus includes kits which include some or all of the reagents necessary to carry out the methods of the present invention, e.g., sample preparation reagents, oligonucleotides, binding molecules, stock solutions, nucleotides, polymerases, inhibitors, enzymes, positive and negative control oligonucleotides and target sequences, test tubes or plates, labeling reagents, fragmentation reagents, detection reagents, purification matrices, and an instruction manual. In some embodiments, the kit of the present invention contains a non-canonical nucleotide. Suitable non-canonical nucleotides include any nucleotides provided herein including but not limited to dUTP, or a methylated purine.

In some embodiments, the kit may contain one or more reaction mixture components, or one or more mixtures of reaction mixture components. In some cases, the reaction mixture components or mixtures thereof may be provided as concentrated stocks, such as 1.1×, 1.5×, 2×, 2.5×, 3×, 4×, 5×, 6×, 7×, 10×, 15×, 20×, 25×, 33×, 50×, 75×, 100× or higher concentrated stock. The reaction mixture components may include any of the compositions provided herein including but not limited to buffers, salts, divalent cations, azeotropes, chaotropes, dNTPs, labeled nucleotides, non-canonical nucleotides, dyes, fluorophores, biotin, enzymes (such as endonucleases, exonucleases, glycosylases), or any combination thereof.

In some embodiments, the kit may contain one or more oligonucleotide primers, such as the oligonucleotide primers provided herein. For example, the kit may contain one or more oligonucleotide primers comprising random hybridizing portions. Alternatively, the kit may contain oligonucleotide primers comprising polyT hybridizing portions. In some cases, the kit may contain oligonucleotide primers that comprise random hybridizing portions and primers comprising polyT hybridizing portions. In still other cases, the kit may contain “not so random” primers that have been pre-selected to hybridize to desired nucleic acids, but not hybridize to undesired nucleic acids. In some cases the kit may contain tailed primers comprising a 3′-portion hybridizable to the target nucleic acid and a 5′-portion which is not hybridizable to the target nucleic acid. In some cases, the kit may contain chimeric primers comprising an RNA portion and a DNA portion. In some cases, the kit may contain primers comprising non-canonical nucleotides.

In some embodiments, the kit of the present invention may contain one or more polymerases or mixtures thereof. In some cases, the one or more polymerases or mixtures thereof may comprise strand displacement activity. Suitable polymerases include any of the polymerases provided herein. The kit may further contain one or more polymerase substrates such as for example dNTPs, non-canonical nucleotides, or labeled nucleotides.

In some embodiments, the kit of the present invention may contain one or more agents capable of inhibiting the DNA-dependent DNA polymerase activity or the RNA-dependent DNA polymerase (reverse transcriptase), such as actinomycin D (dactinomycin), alpha-amanitin, aphidicolin, BPS, novobiocin, rifampicin, rifamycin, sulfoquinovosylmonoacylglycerol, sulfoquinovosyldiacylglycerol, ursane, oleanane triterpenoids, ursolic acid, oleanolic acid, mikanolide, dihydromikanolide, dehydroaltenusin, catapol, taxinine, cephalomanninine, dipeptide alcohols, corylifolin; bakuchiol; resveratrol; Neobavaisoflavone; daidzein; bakuchicin, anacardic acid and oleic acid.

In some embodiments, the kit of the present invention may contain one or more means for purification of the nucleic acid products, removing of the fragmented products from the desired products and removing the inhibitor of DNA-dependent DNA polymerase activity, or combination of the above. Suitable means for the purification of the nucleic acid products include but are not limited to single stranded specific exonucleases, affinity matrices, nucleic acid purification columns, spin columns, ultrafiltration or dialysis reagents, or electrophoresis reagents including but not limited acrylamide or agarose, or any combination thereof.

In some embodiments, the kit of the present invention may contain one or more agents capable of cleaving the base portion of a non-canonical nucleotide to generate an abasic site. In some cases, this agent may comprise one or more glycosylases. Suitable glycosylases include any glycosylases provided herein including but not limited to UDG, or MPG.

In some embodiments, the kit of the present invention may contain one or more agents capable of fragmenting a phosphodiester backbone at an abasic site to fragment the input nucleic acid template. In some cases, this agent may comprise one or more amines, primary amines, secondary amines, polyamines, piperidine, AP endonucleases, or any combination thereof.

In some embodiments, the kit of the present invention may contain one or more compositions or reagents for removing single stranded nucleic acid from the reaction mixture or purifying the double stranded fragments produced in the methods of the present invention. Suitable compositions or reagents include but are not limited to single stranded specific exonucleases, affinity matrices, nucleic acid purification columns, spin columns, ultrafiltration or dialysis reagents, or electrophoresis reagents including but not limited acrylamide or agarose, or any combination thereof.

In some embodiments, the kits of the present invention may contain one or more reagents for end labeling of the primer extension products such as terminal transferases and labeled nucleotides or labeled terminator nucleotides such as dideoxynucleotides or acyclonucleotides.

In some embodiments, the kit of the present invention may contain one or more reagents for producing blunt ends from the double stranded products generated by the extension reaction. For example, the kit may contain one or more of single stranded DNA specific exonucleases including but not limited to exonuclease 1 or exonuclease 7; a single stranded DNA specific endonucleases such as mung bean exonuclease or S1 exonuclease, one or more polymerases such as for example T4 DNA polymerase or Klenow polymerase, or any mixture thereof. Alternatively, the kit may contain one or more single stranded DNA specific exonucleases, endonucleases and one or more polymerases, wherein the reagents are not provided as a mixture. Additionally, the reagents for producing blunt ends may comprise dNTPs.

In some embodiments, the kit of the present invention may contain one or more reagents for preparing the double stranded products for ligation to adaptor molecules. For example, the kit may contain dATP, dCTP, dGTP, dTTP, or any mixture thereof. In some cases, the kit may contain a polynucleotide kinase, such as for example T4 polynucleotide kinase. Additionally, the kit may contain a polymerase suitable for producing a 3′ extension from the blunt ended double stranded DNA fragments. Suitable polymerases are included, for example, exo-Klenow polymerase.

In some embodiments, the kit of the present invention may contain one or more adaptor molecules such as any of the adaptor molecules provided herein. Suitable adaptor molecules include single or double stranded nucleic acid (DNA or RNA) molecules or derivatives thereof, stem-loop nucleic acid molecules, double stranded molecules comprising one or more single stranded overhangs of 1, 2, 3, 4, 5, 6, 7, 8, 9, 10 bases or longer, proteins, peptides, aptamers, organic molecules, small organic molecules, or any adaptor molecules known in the art that may be covalently or non-covalently attached, such as for example by ligation, to the double stranded DNA fragments.

The kit may further contain instructions for the use of the kit. For example, the kit may contain constructions for generating double stranded fragments of nucleic acids useful for large scale analysis including but not limited to e.g., microarray based analysis such as comparative genomic hybridization, highly parallel SNP analysis, pyrosequencing, sequencing by synthesis, sequencing by hybridization, single molecule sequencing, nanopore sequencing, and sequencing by ligation, high density PCR, microarray hybridization, SAGE, digital PCR, and massively parallel Q-PCR; subtractive hybridization; differential amplification; preparation of libraries (including cDNA and differential expression libraries); preparation of an immobilized nucleic acid (which can be a nucleic acid immobilized on a microarray), and characterizing amplified nucleic acid products generated by the methods of the invention, or any combination thereof. In some cases, the kit may contain instructions for providing or generating an input nucleic acid template comprising one or more non-canonical nucleotides. The kit may further contain instructions for mixing the one or more reaction mixture components to generate one or more reaction mixtures suitable for the methods of the present invention. The kit may further contain instructions for hybridizing the one or more oligonucleotide primers to an input nucleic acid template. The kit may further contain instructions for extending the one or more oligonucleotide primers with for example a polymerase, including but not limited to a polymerase comprising strand displacement activity. The kit may further contain instructions for cleaving the base portion of a non-canonical nucleotide to generate an abasic site, with for example a glycosylase. The kit may further contain instructions for fragmenting a phosphodiester backbone at an abasic site to fragment the input nucleic acid template, with for example any of the suitable agents provided herein such as a polyamine. The kit may further contain instructions for purification of any of the products provided by any of the steps of the methods provided herein. The kit may further contain instructions for degradation of single stranded nucleic acids present within the reaction mixture with for example a single stranded DNA specific exonuclease. The kit may further contain instructions for producing blunt ended fragments, for example by removing single stranded overhangs or filling in single stranded overhangs, with for example single stranded DNA specific exonucleases, polymerases, or any combination thereof. The kit may further contain instructions for phosphorylating the 5′ ends of the double stranded DNA fragments produced by the methods of the present invention. The kit may further contain instructions for ligating one or more adaptor molecules to the double stranded DNA fragments of the present invention. The kit may further contain instructions for use of the double stranded DNA fragments for downstream analysis such as hybridization, array hybridization, microarray hybridization, large-scale sequencing, or polymorphism detection.

### V. Examples

### Example 1

### Whole Transcriptome Amplification Followed by Random Priming to Generate dsDNA Products with Reduced (Degradation) of Input Amplified Products

Linear Whole Transcriptome Amplification is carried out using WT-Ovation Pico RNA Amplification kit (NuGEN Technologies Inc, San Carlos) following the manufacturer instructions (http://www.nugeninc.com/tasks/sites/nugen/assets/File/user_guides/userguide_wt_ov_pico.pdf) to generate template nucleic acid. The input total RNA for each reaction is 10 ng of total RNA samples (HeLa total RNA and Brain total RNA from Ambion and total RNA from a biological sample). The amplification is performed in the presence of all four dNTPs as well as dUTP to render the template nucleic acid susceptible to fragmentation by the combined action of UDG and DMED.

The amplified cDNA is purified using QIAquick® PCR Purification Kit, Cat. #28104 as describe in the User Guide (reference above).

Random priming and extension of the amplified cDNA (i.e. template nucleic acid) is carried out using the WT-Ovation Exon Module (http://www.nugeninc.com/nugen/index.cfm/products/target-prep-modules/wt-ovation-exon-module/) with a modified buffer E2. The buffer is prepared with all four dNTPs without dUTP, so as to render the random priming products resistant to fragmentation by the combined action of UDG and DMED. In of WT-Ovation Pico amplified and purified DNA (i.e. template nucleic acid) is combined with 12 μL water, 20 μL of the modified E2 buffer (no dUTP) to a total volume of 32 μL. The mixture is heated at 98° C. for 3 min. and cooled down to 4° C. Following this denaturation step, 3 μL each of random primer and enzyme reagent (E1 and E3) are added to the mixture and the reaction mixture is incubated for the following temperature and durations: 4° C. for 1 min., 30° C. for 10 min., 42° C. for 15 and 75° C. for 10 min. (enzyme inactivation). The primer extension reaction products (including the input template nucleic acid) are purified using Agencourt RNAClean® magnetic beads. The primer extension products (including the input template nucleic acid) are eluted from the beads in 20 μL water. Yield is measured using Nanodrop and electrophoretic mobility is determined using BioAnanlyzer (Agilent).

4-5 μg of purified primer extension products, including the input template nucleic acid comprising dUTP (in 16 μl), is mixed with 3 μL of fragmentation buffer (DMED, NuGEN's FL-Ovation Kit (http://www.nugeninc.com/nugen/index.cfm/products/target-prep-modules/fl-ovation-biotin-v21) and 1 μL of UDG (2 U/μL; NEB). The mixture is incubated at 37° C. for 30 min.

Following fragmentation of the template nucleic acid, the primer extension products are purified using Agencourt RNAClean magnetic beads (following manufacturer instruction) and the purified products are eluted in 20 μL water. Yield and electrophoretic mobility profile are determined as above.

Results: The BioAnalyzer profiles for the linearly amplified DNA, the product of random-primed primer extension reaction on the amplified DNA, and the product of random priming followed by cleavage of the input amplified DNA generated for the various total RNA samples are shown on FIGS. 4 and 5, below.

Amplified cDNA (the product of linear whole transcriptome amplification from the various total RNA samples) generated in the presence of non canonical nucleotide dUTP, can be fragmented by the combined action of UDG and DMED, while the random primed primer extension product generated in the absence of the non canonical nucleotide is not susceptible to cleavage by these combined reagent. Thus, the input amplified cDNA is distinguishable from the random priming products. Cleavage of the input amplified cDNA enables clean-up of the mixture of the random priming products away from the input, as is demonstrated by the reduced size distribution as indicated by the BioAnanlyzer (Agilent) profiles, which reflect change in the length of the multiplicity of species in the analyzed sample.

The sample input on to the Bioanalyzer chips is 1.5 μL containing various amount of DNA: Input amplified cDNA is about 200 ng/μL, random priming product (prior to cleavage step) is about 170 ng/μL and the random priming product following cleavage of the input amplified cDNA is about 100 ng/μL.

### Example 2

### Preparation of Double Stranded Products of the Present Invention for High Throughput Sequencing

The double stranded amplification products of Example 1 are blunt ended using a single stranded exonuclease such as exo1 or exo7 for degrading single stranded overhangs. Alternatively a polymerase such as T4 DNA polymerase or Klenow DNA polymerase is utilized for filling in single stranded overhangs, or a combination of exonuclease and polymerase. The blunt ended double stranded products are then phosphorylated with T4 polynucleotide kinase. The phosphorylated double stranded nucleic acid amplification products are then ligated to adaptor nucleic acids. The amplification products are then encapsulated in a water-in-oil emulsion and amplified in a clonal fashion in the presence of particles comprising sequences complementary to the adaptor nucleic acids. The resulting particles comprising clonally amplified amplification products are then sequenced using a Genome Sequencer manufactured by 454/Roche LifeSciences.

The above-described method may be automated such that the sequencing reactions are performed via robotics. In addition, the sequencing information and data obtained may be provided to a personal computer, a personal digital assistant, a cellular phone, a video game system, or a television so that a user can monitor the progress of the sequencing reactions remotely. This process is illustrated, for example, in FIG. 6. The performing, monitoring and obtaining of results of the sequencing reactions can be done from a place other than where the sequencing apparatus is located, for example from a physically separate room, building, city, state, country or the like. Likewise, the results can be transmitted to the remote user from a physically separate room, building, city, state, country or the like.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method comprising:
(a) providing an input nucleic acid template in a reaction mixture;
(b) hybridizing one or more oligonucleotide primers to the input nucleic acid template;
(c) extending the one or more oligonucleotide primers along the input nucleic acid template with a polymerase comprising strand displacement activity, wherein the hybridizing and extending produces primer extension products comprising one or more double stranded products; and
(d) cleaving the input nucleic acid template with an agent comprising one or more cleavage reagents.

2. The method of claim 1, wherein the input nucleic acid template comprises one or more non-canonical nucleotides.

3. The method of claim 2, wherein the method further comprises generating one or more blunt ended double stranded products from the one or more double stranded products.

4. The method of claim 3 wherein the generating one or more blunt ended double stranded products comprises using an enzyme.

5. The method of claim 4 wherein the enzyme is an exonuclease, an endonuclease, or a combination thereof.

6. The method of claim 5 wherein the enzyme is an endonuclease.

7. The method of claim 6 wherein the endonuclease is S1 endonuclease, mung bean endonuclease, or a combination thereof.

8. The method of claim 6 wherein the method further comprises generating a library of blunt ended double stranded products.

9. The method of claim 6 wherein the method further comprises analyzing the blunt ended double stranded products by next generation sequencing.

10. The method of claim 2 wherein the non-canonical nucleotides comprise dUTP.

11. The method of claim 2, wherein the cleaving step cleaves a base portion of the non-canonical nucleotide, thereby forming an abasic site.

12. The method of claim 2, wherein the agent comprises a glycosylase.

13. The method of claim 12, wherein the glycosylase is UNG or UDG.

14. The method of claim 2, wherein the agent comprises a primary amine.

15. The method of claim 2, wherein the agent comprises a polyamine.

16. The method of claim 15, wherein the polyamine is DMED.

17. The method of claim 2, wherein the agent comprises a glycosylase and a polyamine.

18. The method of claim 1, wherein the input template nucleic acid comprises DNA or RNA, or complements thereof or products of amplification of the input DNA or RNA template.

19. The method of claim 1, wherein steps (b) and (c) are performed simultaneously.

20. The method of claim 1, wherein steps (b) and (c) are performed sequentially.

21. The method of claim 1, wherein the one or more oligonucleotide primers comprise a label.

22. The method of claim 1, wherein the one or more oligonucleotide primers comprise an amino-allyl label.

23. The method of claim 1, wherein the one or more oligonucleotide primers comprise a random hexamer, heptamer, octomer, nonamer, decamer, undecamer, dodecamer, or tridecamer.

24. The method of claim 1, wherein the one or more oligonucleotide primers comprise a poly T sequence.

25. The method of claim 1, wherein the one or more oligonucleotide primers comprise a mixture of random hexamers, heptamers, octomers, nonamers, decamers, undecamers, dodecamers, or tridecamers; and a poly T sequence.

26. The method of claim 1 wherein one or more oligonucleotide primers comprise a template hybridizing portion and a tailed, non template hybridizing, portion.

27. The method of claim 1 wherein one or more oligonucleotide primers comprise chimeric primers.

28. The method of claim 27 wherein the chimeric primers comprise a DNA portion and a 5′-RNA portion.

29. The method of claim 1, wherein the method further comprises degrading single stranded DNA in the reaction mixture.

30. The method of claim 29, wherein the method further comprises degrading single stranded DNA in the reaction mixture with an ssDNA specific exonuclease, an ss DNA specific endonuclease, or a combination thereof.

31. The method of claim 30, wherein the exonuclease is exonuclease 1, exonuclease 7 or a combination thereof.

32. The method of claim 20, wherein the endonuclease is S1 endonuclease, mung bean endonuclease, or a combination thereof.

33. The method of claim 1, wherein the strand displacing polymerase comprises Klenow polymerase, exo-Klenow polymerase, 5′-3′ exo-Klenow polymerase, Bst polymerase, Bst large fragment polymerase, Vent polymerase, Vent polymerase, Deep Vent (exo-) polymerase, 9° Nm polymerase, Therminator polymerase, Therminator II polymerase, MMulV Reverse Transcriptase, phi29 polymerase, or DyNAzyme EXT polymerase, or a combination thereof.

34. The method of claim 1, wherein the method further comprises phosphorylating the 5′ ends of the one or more blunt ended double stranded products.

35. The method of claim 1, wherein the method further comprises extending the 3′ end of the one or more blunt ended double stranded products.

36. The method of claim 35, wherein the method further comprises ligating the double stranded products with one or more double stranded adapter oligonucleotides.

37. The method of claim 1, further comprising subjecting the input nucleic acid template to PCR, strand displacement amplification (SDA), multiple displacement amplification (MDA), rolling circle amplification (RCR), single primer isothermal amplification (SPIA), Ribo-SPIA, ligase chain reaction (LCR), Nucleic Acid Sequence Based Amplification (NASBA), Q-Beta Replicase amplification, Self-sustained sequence replication (3SR) or ligation activated transcription (LAT).

38. A method comprising:
(a) contacting an input template nucleic acid comprising one or more non-canonical nucleotides in a reaction mixture comprising:
(i) one or more oligonucleotide primers;
(ii) a polymerase comprising strand displacement activity;
(iii) an agent capable of cleaving a base portion of a non-canonical nucleotide, whereby an abasic site is generated; and
(iv) an agent capable of fragmenting a phosphodiester backbone at the abasic site; whereby double stranded DNA fragments are generated.

39. A method comprising:
(a) providing an input nucleic acid template comprising one or more non-canonical nucleotides;
(b) amplifying the input nucleic acid template to produce amplification products in a reaction mixture comprising oligonucleotide primers comprising random hybridizing portions, and an enzyme comprising strand displacement activity; and
(c) fragmenting the input nucleic acid template, wherein the fragmenting step is performed by adding to the reaction mixture comprising amplification products an agent that cleaves a base portion of a non-canonical nucleotide to generate an abasic site and an agent that cleaves a phosphodiester backbone of a nucleic acid at an abasic site.

40. The method of claim 38 further comprising:
(d) optionally separating the amplification products from the fragmentation products;
(e) treating the amplification products with one or more agents to produce double stranded blunt-ended amplification products; and
(f) phosphorylating the 5′ ends of the double stranded blunt-ended amplification products.

41. The method of claim 40, wherein step (d) is performed after step (f).

42. The method of claim 40, wherein step (d) is performed before step (e).

43. The method of claim 40, wherein step (d) is not performed.

44. The method of claim 40 further comprising ligating the one or more phosphorylated double stranded blunt-ended amplification products with adaptor nucleic acid molecules.

45. The method of claim 44, wherein the amplification products are analyzed by next generation sequencing.

46. A kit comprising:
(a) a glycosylase;
(b) a polyamine, an AP endonuclease, or a combination thereof; and
(c) one or more double stranded adapter oligonucleotides.

47. The kit of claim 46, wherein the kit further comprises instructions for the use of said kit.

48. The kit of claim 47, wherein the instructions comprise a method for generating dsDNA fragments, said method comprising:
(a) providing an input nucleic acid template comprising one or more non-canonical nucleotides in a reaction mixture;
(b) hybridizing and extending one or more oligonucleotide primers to the input nucleic acid template with an enzyme comprising strand displacement activity, wherein the hybridizing and extending produces one or more double stranded products; and
(c) cleaving the input nucleic acid template.

49. The kit of claim 46, wherein the kit further comprises one or more oligonucleotide primers.

50. The kit of claim 49, wherein the one or more oligonucleotide primers comprise random hybridizing portions.

51. The kit of claim 49, wherein the one or more oligonucleotide primers comprise polyT hybridizing portions.

52. The kit of claim 49, wherein the one or more oligonucleotide primers comprise a mixture of random hybridizing portions and polyT portions.

53. The kit of claim 49, wherein at least one of the one or more oligonucleotide primers is a composite primer.

54. The kit of claim 49, wherein at least two of the one or more oligonucleotide primers is a composite primer.

55. A composition comprising:
(a) an input template nucleic acid comprising one or more non-canonical nucleotides;
(b) one or more oligonucleotide primers comprising randomized hybridizing portions;
(c) a polymerase comprising strand-displacement activity;
(d) a glycosylase; and
(e) one or more double stranded DNA product molecules.

56. A kit comprising:
(a) an input template nucleic acid;
(b) one or more oligonucleotide primers comprising randomized hybridizing portions;
(c) a polymerase comprising strand-displacement activity; and
(d) an endonuclease.

57. A method comprising:
(a) providing an input RNA template in a reaction mixture;
(b) hybridizing a first primer to the input RNA template;
(c) reverse transcribing the input RNA template in the presence of one or more non-canonical nucleotides to generate a first strand cDNA;
(d) cleaving the input RNA template;
(e) performing second strand synthesis to generate a double stranded cDNA; and
(f) cleaving the first strand cDNA to generate a single stranded second strand cDNA.

58. The method of claim 57, wherein said reverse transcribing comprises use of an RNA-dependent DNA polymerase.

59. The method of claim 58, wherein the reaction mixture further comprises an inhibitor of DNA-dependent DNA polymerase activity.

60. The method of claim 59, wherein the inhibitor of DNA-dependent DNA polymerase activity inhibits the DNA-dependent DNA polymerase activity of the RNA-dependent DNA polymerase.

61. The method of claim 59, wherein said inhibitor is actinomycin D.

62. The method of claim 59, wherein said inhibitor is removed prior to the performing second strand synthesis.

63. The method of claim 57, wherein the first primer comprises a 5′-tail sequence that does not hybridize to the input RNA template.

64. The method of claim 63, wherein the 5′-tail sequence comprises DNA.

65. The method of claim 57, wherein the 3′-end of the first primer hybridizes to the input RNA template.

66. The method of claim 57, wherein the reverse transcribing comprises extension of the first primer hybridized to the input RNA template.

67. The method of claim 57, wherein the non-canonical nucleotide is dUTP.

68. The method of claim 57, wherein said second strand synthesis comprises primer extension of a second primer hybridized to the first strand cDNA.

69. The method of claim 68, wherein said second primer comprises a 3′-sequence hybridizable to the first strand cDNA.

70. The method of claim 68, wherein said second primer comprises a 5′-tail that is not complementary to the first strand cDNA.

71. The method of claim 57, wherein said second strand synthesis is carried out by DNA polymerase.

72. The method of claim 57, wherein said second strand synthesis is carried out in the absence of non-canonical nucleotide triphosphates.

73. The method of claim 57, wherein cleaving the input RNA template in a complex with the first primer extension product or products comprises exposing the input RNA template to an RNase H with or without other RNases, or cleaving the input RNA template following first primer extension reaction by heat or chemical treatment or combination thereof.

74. The method of claim 57, wherein cleaving the first strand cDNA comprises combining the reaction mixture with an enzyme that cleaves the base portion of the non-canonical nucleotide to generate an abasic site.

75. The method of claim 74, wherein said enzyme that cleaves the base portion of the non-canonical nucleotide to generate an abasic site is a glycosylase.

76. The method of claim 74, wherein said glycosylase is UNG or UDG.

77. The method of claim 74, wherein the reaction mixture further comprises an amine.

78. The method of claim 77, wherein said amine is DMED.

79. The method of claim 57, wherein said cleaving the first strand cDNA generates fragments of the first strand cDNA with blocked 3′-ends.

80. The method of claim 57, further comprising sequencing said single stranded second strand cDNA.

81. The method of claim 80, wherein said sequencing comprises next generation sequencing.

82. The method of claim 57, wherein said single stranded second strand cDNA comprises sequence homologous to the input RNA template flanked by 3′ and/or 5′ sequences compatible for use in DNA sequencing.

83. The method of claim 57, wherein said single stranded second strand cDNA comprises sequence homologous to the input RNA template flanked by 3′ and/or 5′ comprising sequences that function as barcodes.

84. The method of claim 57, wherein said single stranded second strand cDNA comprises sequence homologous to the input RNA template flanked by 3′ and/or 5′ sequences that comprise recognition sequence for one or more restriction enzymes.

85. The method of claim 57, wherein said single stranded second strand cDNA comprises sequence homologous to the input RNA template flanked by 3′ and/or 5′ sequences that enables circularization by hybridizing an oligonucleotide complementary to the end sequences and ligation of the ends.

86. The method of claim 57, further comprising amplification of the cDNA by rolling circle amplification.

87. The method of claim 57, further comprising amplifying the single stranded cDNA by single primer isothermal amplification.

88. The method of claim 57, wherein the input RNA template is from a biological sample.

89. The method of claim 57, wherein the input RNA template is from a sample lysate.

90. The method of claim 57, wherein the input RNA template is from a cell free fluid.

91. The method of claim 57, wherein the cell free fluid is plasma or serum.

92. The method of claim 57, wherein the input RNA template is fragmented RNA.

93. The method of claim 92, wherein the input RNA template is from an FFPE sample.

94. The method of claim 92, wherein the input RNA template is fragmented by treatment with heat in the presence of multivalent cations.
